(19) United States
(12) Patent Application Publication
(10) Pub. No.: US 2025/0207153 A1
(43) Pub. Date: Jun. 26, 2025

(54) INTEGRATION OF LARGE NUCLEIC ACIDS INTO GENOMES

(71) Applicant: THE REGENTS OF THE UNIVERSITY OF CALIFORNIA, Oakland, CA (US)

(72) Inventors: Patrick Hsu, Berkeley, CA (US); Matthew Durrant, Berkeley, CA (US); Alison Fanton, Berkeley, CA (US); Chad Moon, Midlothian, VA (US)

(21) Appl. No.: 18/846,744

(22) PCT Filed: Nov. 3, 2022
(86) PCT No.: PCT/US2022/048841

§ 371 Date: Sep. 13, 2024

Related U.S. Application Data
(60) Provisional application No. 63/269,299, filed on Mar. 14, 2022.
(60) Provisional application No. 63/370,543, filed on Aug. 5, 2022.

(51) Int. Cl. C12N 15/90; C12N 9/22; C12N 15/11

(52) U.S. Cl. CPC C12N 15/907; C12N 9/22; C12N 15/11; C12N 2310/20

(57) ABSTRACT

This document relates to compositions, methods, and systems for site-specific integration (e.g., stable integration) of a nucleic acid (e.g., large nucleic acid) into the genome of a cell (e.g., a prokaryotic cell or a eukaryotic cell such as a plant cell or an animal cell). For example, compositions, methods, and systems for stably integrating one or more nucleic acids into a target site within the genome of a cell that include (a) a genome-editing system having (i) a polypeptide having a DNA binding domain and, optionally, a polymerase and (ii) a nucleic acid molecule including a guide sequence that is complementary to the target site and a nucleic acid sequence that encodes an acceptor attachment (attA) site, (b) a donor nucleic acid molecule including a nucleic acid cargo and a donor attachment (attD) site, and (c) an integrase (e.g., a large serine recombinase (LSR)) that can target the attA site and the attD site, where the integrase can facilitate recombination between the attA site and the attD site are provided.

### STATEMENT REGARDING FEDERAL FUNDING

This invention was made with government support under OD021369 awarded by the National Institutes of Health. The government has certain rights in the invention.

### TECHNICAL FIELD

This document relates to compositions, methods, and systems for site-specific integration (e.g., stable integration) of a nucleic acid (e.g., large nucleic acid) into the genome of a cell (e.g., a prokaryotic cell or a eukaryotic cell such as a plant cell or an animal cell). For example, this document provides compositions, methods, and systems for stably integrating one or more nucleic acids into a target site within the genome of a cell that include (a) a genome-editing system having (i) a polypeptide having a DNA binding domain and, optionally, a polymerase and (ii) a nucleic acid molecule including a guide sequence that is complementary to the target site and a nucleic acid sequence that encodes an acceptor attachment (attA) site, (b) a donor nucleic acid molecule including a nucleic acid cargo and a donor attachment (attD) site, and (c) an integrase (e.g., a large serine recombinase (LSR)) that can target the attA site and the attD site, where the integrase can facilitate recombination between the attA site and the attD site.

### BACKGROUND INFORMATION

Current gene integration approaches rely on DNA double-stranded breaks (DSBs) to direct cellular DNA repair pathways such as homologous recombination (HR). These approaches generally suffer from low insertion efficiency, high indel rates, and cargo size limitations. Additional gene integration approaches such as transposase-mediated integration and lentiviral-mediated integration are not site-specific, and can result in variable gene expression, silenced gene expression, insertional mutagenesis, and/or other undesired events

Despite the recent advances in genome engineering technologies, there remains a need for an efficient method to stably and site-specifically integrate multi-kilobase DNA cargos into human and other eukaryotic cell genomes.

### SUMMARY

This document provides compositions, methods, and systems for integrating (e.g., stably integrating) nucleic acid (e.g., large nucleic acid) into the genome of a cell (e.g., prokaryotic cell or a eukaryotic cell such as a plant cell or an animal cell). For example, this document provides compositions, methods, and systems for stably integrating one or more nucleic acids into a target site within the genome of a cell that include (a) a genome-editing system having (i) a polypeptide having a DNA binding domain and, optionally, a polymerase and (ii) a nucleic acid molecule including a guide sequence that is complementary to the target site and a nucleic acid sequence that encodes an attA site, (b) a donor nucleic acid molecule including a nucleic acid cargo and an attD site, and (c) an integrase (e.g., a LSR) that can target the attA site and the attD site. For example, when a genome-editing system provided herein is administered to a cell, the genome-editing system can insert the attA into the genome at the target site, and the integrase can facilitate recombination between the attA site and the attD site thereby integrating the donor nucleic acid molecule into the genome.

As demonstrated herein, a genome-editing system (e.g., a prime-editor system) can be used together with an integrase (e.g., a LSR) to stably integrate multi-kilobase DNA cargos into human and other eukaryotic cell genomes. The compositions, methods, and systems provided herein not only provide precise control over the genomic integration site (thus reducing or eliminating the risk of insertional mutagenesis), but can allow the site-specific integration of large (e.g., multi-kilobase) nucleic acid cargos into the genome. The compositions, methods, and systems provided herein can be applied to any appropriate gene editing application including, without limitation, gene therapy methods, gene transfer methods, production of transgenic plants, production of gene knock-out plants, and production of gene knock-out non-human animal models.

In general, one aspect of this document features systems for stably integrating one or more nucleic acid sequences into a genome of a cell. The systems can include, or consist essentially of, administering to a cell: (a) a genome-editing system that can insert an attA sequence into a target site within a genome of the cell; (b) a donor nucleic acid molecule comprising a nucleic acid cargo and a attD sequence; and (c) an integrase that targets the attA sequence and the attD site and can facilitate recombination between the attA site and the attD site. The cell can be a mammalian cell (e.g., a human cell). The cell can be a plant cell. The cell can be a prokaryotic cell. The genome-editing system can include (i) a polypeptide comprising a DNA binding domain and (ii) a nucleic acid comprising a guide sequence that is complementary to the target site within the genome and a sequence that encodes the attA sequence. The DNA binding domain can be present in polypeptide selected from a Cas9 polypeptide,a Cas12 polypeptide, a zinc finger polypeptide, and a transcription activator-like effector (TALE) polypeptide. The polypeptide including the DNA binding domain can be a polymerase. The polymerase can be a reverse transcriptase (RT) selected from the group consisting of a Moloney murine leukemia virus (M-MLV) RT, an avian myeloblastosis virus (AMV) RT, and a human immunodeficiency virus type 1 (HIV-1) RT. The attA sequence can include from about 20 to about 100 nucleic acids. The attA sequence can comprise, consist essentially of, or consist of any one of SEQ ID NOs: 11-84 and SEQ ID NO:254. The attD sequence can include from about 20 to about 100 nucleic acids. The attD sequence can comprise, consist essentially of, or consist of any one of SEQ ID NOs: 159-232. The integrase can be a LSR. The LSR can have an amino acid sequence containing a motif set forth in any one of SEQ ID NOs: 233-245. The LSR can have an amino acid sequence having at least 70% sequence identity to the sequence of any one of SEQ ID NOs:85-158. The LSR can have an amino acid sequence having at least 90% sequence identity to the sequence of any one of SEQ ID NOs:85-158. The LSR can comprise, consist essentially of, or consist of an amino acid sequence set forth in any one of SEQ ID NOs:85-158. The donor nucleic acid molecule can be from about 250 nt to about 30 kb.

In another aspect, this document features methods for stably integrating one or more nucleic acid sequences into a genome of a cell. The methods can include, or consist essentially of, administering to a cell: (a) a genome-editing system that can insert an attA sequence into a target site within a genome of the cell; (b) a donor nucleic acid molecule comprising a nucleic acid cargo and an attD sequence; and (c) an integrase that targets the attA sequence and the attD site; where the genome-editing system integrates the attA sequence into the target site, and where the integrase facilitates recombination between the attA sequence and the attD sequence thereby integrating the donor nucleic acid molecule into the genome of the cell. The cell can be a T cell, a natural killer (NK) cell, a non-human embryonic stem cell, an induced pluripotent stem cell (iPSC), a hematopoietic stem cell (HSC), a liver cell, a muscle cell, a monocytes, a B cell, a neuron, an astrocyte, or a microglial cell. The cell can be a T cell and the nucleic acid sequence can encode a chimeric antigen receptor polypeptide or an engineered T cell receptor. The cell is a NK cell and the nucleic acid sequence can encode a T cell receptor or an engineered natural killer cell receptor. The cell can be a mammalian cell (e.g., a human cell). The cell can be a plant cell. The genome-editing system can include (i) a polypeptide comprising a DNA binding domain and (ii) a nucleic acid comprising a guide sequence that is complementary to the target site within the genome and a sequence that encodes the attA sequence. The DNA binding domain can be present in a polypeptide selected from a Cas9 polypeptide, a Cas12 polypeptide, a zinc finger polypeptide, and a TALE polypeptide. The polypeptide comprising the DNA binding domain can be a polymerase. The polymerase can be an RT selected from the group consisting of a M-MLV RT, an AMV RT, and a HIV-1 RT. The attA sequence can comprise, consist essentially of, or consist of any one of SEQ ID NOs: 11-84 and SEQ ID NO:254. The attD sequence can comprise, consist essentially of, or consist of any one of SEQ ID NOs: 159-232. The integrase can be a LSR. The LSR can have an amino acid sequence containing a motif set forth in any one of SEQ ID NOs:233-245. The LSR can have an amino acid sequence having at least 70% sequence identity to the sequence of any one of SEQ ID NOs:85-158. The LSR can have an amino acid sequence having at least 90% sequence identity to the sequence of any one of SEQ ID NOs:85-158. The LSR can comprise, consist essentially of, or consist of an amino acid sequence set forth in any one of SEQ ID NOs:85-158.

In another aspect, this document features methods for labelling a polypeptide encoded by an endogenous nucleic acid within a cell. The methods can include, or consist essentially of, administering to a cell: (a) a genome-editing system that can insert an attA sequence into a target site within a genome of the cell; (b) a donor nucleic acid molecule comprising a nucleic acid cargo encoding a detectable label and an attD sequence; and (c) an integrase that targets the attA sequence and the attD site; where the genome-editing system integrates the attA sequence into the target site, and where the integrase facilitates recombination between the attA sequence and the attD sequence thereby integrating the donor nucleic acid molecule into the genome of the cell such that the cell expresses a fusion polypeptide including the polypeptide encoded by the endogenous nucleic acid fused to the detectable label. The detectable label can be a HiBiT tag, a HaloTag, a Flag tag, a HA tag, a MS2/PP7 tag, a Sun/Moon tag, a poly(His) tag, a mCherry polypeptide, a green fluorescent polypeptide (GFP), a glutathione-S-transferase (GST), a luciferase, a horseradish peroxidase (HRP), an alkaline phosphatase (AP), or a apurinic/apyrimidinic endodeoxyribonuclease 2 (APEX2) polypeptide. The cell can be a mammalian cell (e.g., a human cell). The cell can be a plant cell. The genome-editing system can include (i) a polypeptide comprising a DNA binding domain and (ii) a nucleic acid comprising a guide sequence that is complementary to the target site within the genome and a sequence that encodes the attA sequence. The DNA binding domain can be present in a polypeptide selected from a Cas9 polypeptide, a Cas12 polypeptide, a zinc finger polypeptide, and a TALE polypeptide. The polypeptide including the DNA binding domain can be a polymerase. The polymerase can be a RT selected from the group consisting of a M-MLV RT, an AMV RT, and a HIV-1 RT. The attA sequence can comprise, consist essentially of, or consist of any one of SEQ ID NOs: 11-84 and SEQ ID NO:254. The attD sequence can comprise, consist essentially of, or consist of any one of SEQ ID NOs: 159-232. The integrase can be a LSR. The LSR can have an amino acid sequence containing a motif set forth in any one of SEQ ID NOs:233-245. The LSR can have an amino acid sequence having at least 70% sequence identity to the sequence of any one of SEQ ID NOs:85-158. The LSR can have an amino acid sequence having at least 90% sequence identity to the sequence of any one of SEQ ID NOs:85-158. The LSR can comprise, consist essentially of, or consist of an amino acid sequence set forth in any one of SEQ ID NOs:85-158.

In another aspect, this document features methods for making a non-human transgenic organism. The methods can include, or consist essentially of, administering to an embryonic stem cell of a non-human organism: (a) a genome-editing system that can insert an attA sequence into a target site within a genome of the embryonic stem cell; (b) a donor nucleic acid molecule comprising a transgene and an attD sequence; and (c) an integrase that targets the attA sequence and the attD site; where the genome-editing system integrates the attA sequence into the target site, and where the integrase facilitates recombination between the attA sequence and the attD sequence thereby integrating the donor nucleic acid molecule into the genome of the cell such that the cell expresses the transgene. The cell can be a non-human mammalian cell. The cell can be a plant cell. The transgene expressed by the plant cell can be a herbicide resistance polypeptide. The genome-editing system can include (i) a polypeptide comprising a DNA binding domain and (ii) a nucleic acid comprising a guide sequence that is complementary to the target site within the genome and a sequence that encodes the attA sequence. The DNA binding domain can be present in a polypeptide selected from a Cas9 polypeptide, a Cas12 polypeptide, a zinc finger polypeptide, and a TALE polypeptide. The polypeptide including the DNA binding domain can be a polymerase. The polymerase can be an RT selected from the group consisting of a M-MLV RT, an AMV RT, and a HIV-1 RT. The attA sequence can comprise, consist essentially of, or consist of any one of SEQ ID NOs:11-84 and SEQ ID NO:254. The attD sequence can comprise, consist essentially of, or consist of any one of SEQ ID NOs: 159-232. The integrase can be a LSR. The LSR can have an amino acid sequence containing a motif set forth in any one of SEQ ID NOs:233-245. The LSR can have an amino acid sequence having at least 70% sequence identity to the sequence of any one of SEQ ID NOs:85-158. The LSR can have an amino acid sequence having at least 90% sequence identity to the sequence of any one of SEQ ID NOs:85-158. The LSR can comprise, consist essentially of, or consist of an amino acid sequence set forth in any one of SEQ ID NOs:83-158.

In another aspect, this document features methods for making a non-human organism having reduced or eliminated levels of a polypeptide. The methods can include, or consist essentially of, administering to an embryonic cell of a non-human organism: (a) a genome-editing system that can insert an attA sequence into a target site within a genome of the cell; (b) a donor nucleic acid molecule comprising a nucleic acid cargo and an attD sequence; and (c) an integrase that targets the attA sequence and the attD site; where the genome-editing system integrates the attA sequence into the target site, and where the integrase facilitates recombination between the attA sequence and the attD sequence thereby integrating the donor nucleic acid molecule into the genome of the cell such that the endogenous nucleic acid sequence encoding the polypeptide is interrupted and expression of the polypeptide is reduced or eliminated. The nucleic acid cargo can include a stop codon. The nucleic acid cargo can include a nucleic acid encoding a selectable marker. The nucleic acid cargo can include nucleic acid encoding a detectable label. The cell can be a non-human mammalian cell. The cell can be a plant cell. The genome-editing system can include (i) a polypeptide comprising a DNA binding domain and (ii) a nucleic acid comprising a guide sequence that is complementary to the target site within the genome and a sequence that encodes the attA sequence. The DNA binding domain can be present in a polypeptide selected from a Cas9 polypeptide, a Cas12 polypeptide, a zinc finger polypeptide, and a TALE polypeptide. The polypeptide including the DNA binding domain can be a polymerase. The polymerase can be an RT selected from the group consisting of a M-MLV RT, an AMV RT, and a HIV-1 RT. The attA sequence can comprise, consist essentially of, or consist of any one of SEQ ID NOs:11-84 and SEQ ID NO:254. The attD sequence can comprise, consist essentially of, or consist of any one of SEQ ID NOs: 159-232. The integrase can be a LSR. The LSR can have an amino acid sequence containing a motif set forth in any one of SEQ ID NOs:233-245. The LSR can have an amino acid sequence having at least 70% sequence identity to the sequence of any one of SEQ ID NOs:85-158. The LSR can have an amino acid sequence having at least 90% sequence identity to the sequence of any one of SEQ ID NOs:85-158. The LSR can comprise, consist essentially of, or consist of an amino acid sequence set forth in any one of SEQ ID NOs:85-158.

In another aspect, this document features methods for treating a mammal having a disease or disorder. The methods can include, or consist essentially of, administering to a mammal having a disease or disorder: (a) a genome-editing system that can insert an attA sequence into a target site within a genome of a cell within the mammal; (b) a donor nucleic acid molecule comprising a nucleic acid cargo encoding a therapeutic gene product and a attD sequence; and (c) an integrase that targets the attA sequence and the attD site; where the genome-editing system integrates the attA sequence into the target site, and where the integrase facilitates recombination between the attA sequence and the attD sequence thereby integrating the donor nucleic acid molecule into the genome of the cell such that the cell produces the therapeutic gene product. The therapeutic polypeptide can be an adenosine deaminase polypeptide, an α-1 antitrypsin polypeptide, a cystic fibrosis transmembrane conductance regulator (CFTR) polypeptide, a β-hemoglobin (HBB) polypeptide, an oculocutaneous albinism II (OCA2) polypeptide, a Huntingtin (HTT) polypeptide, a dystrophia myotonica-protein kinase (DMPK) polypeptide, a low-density lipoprotein receptor (LDLR) polypeptide, an apolipoprotein B (APOB) polypeptide, a neurofibromin 1 (NF1) polypeptide, a polycystic kidney disease 1 (PKD1) polypeptide, a polycystic kidney disease 2 (PKD2) polypeptide, a coagulation factor VIII (F8) polypeptide, a dystrophin (DMD) polypeptide, a phosphate-regulating endopeptidase homologue X-linked (PHEX) polypeptide, a methyl-CpG-binding protein 2 (MECP2) polypeptide, a ubiquitin-specific peptidase 9Y, Y-linked (USP9Y) polypeptide, a carbamoyl-phosphate synthase 1 (CPS1) polypeptide, an ATP binding cassette subfamily A member 4 (ABCA4) polypeptide, an fatty acid elongase 4 (ELOVL) polypeptide, amyosin VIIA (MY07A) polypeptide, an usher syndrome 1C (USH1C) polypeptide, a cadherin related 23 (CDH23) polypeptide, a protocadherin related 15 (PCDH15) polypeptide, an usher syndrome 1G (USH1G) polypeptide, an usher syndrome 2A (USH2A) polypeptide, an adhesion G protein-coupled receptor V1 (ADGRV1) polypeptide, a whirlin (WHRN) polypeptide, a clarin 1 (CLRN1) polypeptide, a retinitis pigmentosa 1 (RP1) polypeptide, an eyes shut homolog (EYS) polypeptide, a lipoprotein (a) (LPA) polypeptide, a lipoprotein lipase (LPL) polypeptide, an apolipoprotein C2 (APOC2) polypeptide, an apolipoprotein A5 (APOA5) polypeptide, a lipase maturation factor 1 (LMF1) polypeptide, a glycosylphosphatidylinositol anchored high density lipoprotein binding protein 1 (GPIHBP1) polypeptide, a proprotein convertase subtilisin/kexin type 9 (PCSK9) polypeptide, a ryanodine receptor 2 (RYR2) polypeptide, a calsequestrin 2 (CASQ2) polypeptide, a myosin heavy chain 7 (MYH7) polypeptide, a myosin binding protein C3 (MYBPC3) polypeptide, a troponin T2, cardiac type (TNNT2) polypeptide, and a troponin 13, cardiac type (TNNI3) polypeptide, or a C9orf72 polypeptide. The mammal can be a human. The genome-editing system can include (i) a polypeptide comprising a DNA binding domain and (ii) a nucleic acid comprising a guide sequence that is complementary to the target site within the genome and a sequence that encodes the attA sequence. The DNA binding domain can be present in a polypeptide selected from a Cas9 polypeptide, a Cas12 polypeptide, a zinc finger polypeptide, and a TALE polypeptide. The polypeptide including the DNA binding domain can be a polymerase. The polymerase can be an RT selected from the group consisting of a M-MLV RT, an AMV RT, and a HIV-1 RT. The attA sequence can comprise, consist essentially of, or consist of any one of SEQ ID NOs:11-84 and SEQ ID NO:254. The attD sequence can comprise, consist essentially of, or consist of any one of SEQ ID NOs: 159-232. The integrase can be a LSR. The LSR can have an amino acid sequence containing a motif set forth in any one of SEQ ID NOs:233-245. The LSR can have of an amino acid sequence having at least 70% sequence identity to the sequence of any one of SEQ ID NOs:85-158. The LSR can have an amino acid sequence having at least 90% sequence identity to the sequence of any one of SEQ ID NOs:85-158. The LSR can comprise, consist essentially of, or consist of an amino acid sequence set forth in any one of SEQ ID NOs:85-158.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. Schematic images of mechanism for using a prime editor in combination with a LSR for programmable recombination of multiple kilobase cargo into the genome. FIG. 1A contains a schematic for using prime editing with a LSR supplied independently (e.g., in trans). FIG. 1B contains a schematic for using prime editing with integrase supplied fused to a component of a prime editor complex (e.g., in cis). FIG. 1C contains a schematic image showing guided delivery of the prime editor to a nucleic acid target site using pegRNA & ngRNA (left) or using two twinPE pegRNAs (right).

FIGS. 2A-2B. Schematic images of exemplary methods for using a prime editor in combination and a LSR in trans for programmable recombination of multiple kilobase cargo into the genome. FIG. 2A contains a schematic of an exemplary method for a one-step transfection to deliver a prime editing system and a LSR to cells. FIG. 2B contains a schematic of an exemplary method for a two-step transfection to deliver a prime editing system and a LSR to cells.

FIG. 3. Sequencing results demonstrating that prime editing can be used for targeted insertion of an attA site. Sequencing results of Bxb1 are, from top to bottom, SEQ ID NOs:246 to 249. Sequencing results of Pa01 are, from top to bottom, SEQ ID NOs:250 and 251.

FIG. 4. PCR validation of donor integration at an attA site.

FIGS. 5A-5B. Sequencing results demonstrating site-specific donor integration. FIG. 5A contains results using a Bxb1 LSR (SEQ ID NO:252). FIG. 5B contains results using a Pa01 LSR (SEQ ID NO:253).

FIG. 6. Evaluation of attA length. Truncations of an exemplary minimal attB site (SEQ ID NO:254) are shown.

FIG. 7. qPCR analysis showing donor integration using 1 pegRNA.

FIGS. 8A-8B. ddPCR analysis showing donor integration. FIG. 8A. Donor integration at the LMNB1 locus using 1 pegRNA. FIG. 8B. Donor integration at the ACTB locus using 1 pegRNA.

FIG. 9. qPCR analysis showing donor integration using 2 pegRNAs at the AAVS1 locus.

FIG. 10. ddPCR analysis showing donor integration at the AAVS1 locus using 2 pegRNAs and LSR delivery in trans.

### DETAILED DESCRIPTION

This document provides compositions, methods, and systems for integrating (e.g., stably integrating) nucleic acid (e.g., large nucleic acid) into the genome of a cell (e.g., a prokaryotic cell or a eukaryotic cell such as a plant cell or an animal cell). For example, this document provides systems for stably integrating one or more nucleic acids into a target site within the genome of a cell that include (a) a genome-editing system having (i) a polypeptide having a DNA binding domain and, optionally, a polymerase and (ii) a nucleic acid molecule including a guide sequence that is complementary to the target site and a nucleic acid sequence that encodes an attA site, (b) a donor nucleic acid molecule including a nucleic acid cargo and an attD site, and (c) an integrase (e.g., a LSR) that can target the attA site and the attD site. For example, when a genome-editing system provided herein is administered to a cell, the genome-editing system can insert the attA into the genome at the target site, and the integrase can facilitate recombination between the attA site and the attD site thereby integrating the donor nucleic acid molecule into the genome.

The compositions, methods, and systems provided herein (e.g., a system for stably integrating one or more nucleic acids into a target site within the genome of a cell including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used to integrate (e.g., stably integrate) a nucleic acid into a genomes of any appropriate type of cell. In some cases, the compositions, methods, and systems provided herein can be used to integrate nucleic acid (e.g., large nucleic acid) into a prokaryotic cell. In some cases, the compositions, methods, and systems provided herein can be used to integrate nucleic acid (e.g., large nucleic acid) into a eukaryotic cell. Examples of cell types that can have a nucleic acid stably integrated within the genome as described herein include, without limitation, stem cells (e.g., non-human embryonic stem cells, induced pluripotent stem cells (iPSCs), and hematopoietic stem cells (HSCs)), immune cells (e.g., T cells, macrophages, monocytes, B cells, and natural killer (NK) cells), liver cells, muscle cells, and brain cells (e.g., neurons, astrocytes, and microglia). For example, a system including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used to integrate (e.g., stably integrate) a nucleic acid into a plant cell or a mammalian cell. Examples of plants whose cells can have a nucleic acid stably integrated into a target site within the genome as described herein include, without limitation, wheat, corn, soy, rice, tobacco, Arabidopsis thaliana, cacao, banana, and sunflower. Examples of mammals whose cells can have a nucleic acid stably integrated into a target site within the genome as described herein include, without limitation, humans, non-human primates such as chimpanzees and monkeys, dogs, cats, horses, cows, pigs, sheep, mice, rats, rabbits, guinea pigs, birds, fish (e.g., zebrafish (Danio rerio), medaka (Oryzias latipes), and turquoise killifish (Nothobranchius furzeri)), nematodes (e.g., Caenorhabditis elegans), and flies (e.g., Drosophila melanogaster).

A genome-editing system in a system for stably integrating one or more nucleic acids into a target site within the genome of a cell provided herein can include (i) a polypeptide having a DNA binding domain and, optionally, a polymerase and (ii) a nucleic acid molecule including a guide sequence that is complementary to the target site and a nucleic acid sequence that encodes an attA site. A polypeptide having a DNA binding domain and, optionally, a polymerase can include any appropriate DNA binding domain. In some cases, a DNA binding domain can be included in a polypeptide including a DNA binding domain. For example, a DNA binding domain can be included in a polypeptide including a DNA binding domain and including nuclease activity. For example, a DNA binding domain can be included in a polypeptide including a DNA binding domain and including nickase activity.

A DNA binding domain can be included in any appropriate polypeptide having nuclease activity. Examples of nucleases include, without limitation, clustered regularly interspaced short palindromic repeat (CRISPR)-associated (Cas) polypeptides, zinc-finger nucleases (ZFNs), and transcription activator-like effector (TALE) polypeptides. In some cases, a nuclease can be as described elsewhere (see, e.g., Urnov and Rebar, Biochem. Pharmacol., 64 (5-6): 919-23 (2002); and Miller et al., Nat. Biotechnol., 29 (2): 143-8 (2011)).

In some cases, a DNA binding domain can be included a Cas polypeptide. A Cas polypeptide can be any appropriate Cas polypeptide. In some cases, a Cas polypeptide can be isolated from an organism (e.g., a bacterium). In some cases, a Cas polypeptide can be a recombinant polypeptide. In some cases, a Cas polypeptide can be a synthetic polypeptide. Examples of Cas polypeptides include, without limitation, Cas9 polypeptides (e.g., a Cas9 nuclease or a Cas9 nickase) such as Cas9 polypeptides from Streptococcus pyogenes (SpCas9 polypeptides) and Cas9 polypeptides from Staphylococcus aureus (SaCas9 polypeptides), Cas12 polypeptides (e.g., a Cas12 nuclease or a Cas12 nickase).

A Cas polypeptide having a DNA binding domain can have any appropriate amino acid sequence. Examples of Cas polypeptide sequences include, without limitation, amino acid sequences set forth in SEQ ID NO:1, SEQ ID NO:2, SEQ ID NO:3, SEQ ID NO:4, SEQ ID NO:5, or SEQ ID NO:6. In some cases, a Cas polypeptide having a DNA binding domain can have one or more amino acid modifications (e.g., one or more insertions, one or more deletions, and/or one or more substitutions) relative to a Cas polypeptide described herein (e.g., SEQ ID NO:1, SEQ ID NO:2, SEQ ID NO:3, SEQ ID NO:4, SEQ ID NO:5, and SEQ ID NO:6), provided the Cas polypeptide maintains the ability to cleave nucleic acid (e.g., maintains its nuclease activity and/or its nickase activity). In some cases, a Cas polypeptide having a DNA binding domain can have at least 70% (e.g., 70%, 75%, 80%, 85%, 90%, 93%, 95%, 96%, 97%, 98%, or 99%) sequence identity to any one of SEQ ID NO:1, SEQ ID NO:2, SEQ ID NO:3, SEQ ID NO:4, SEQ ID NO:5, and SEQ ID NO:6, provided the Cas polypeptide maintains the ability to cleave nucleic acid (e.g., maintains its nuclease activity and/or its nickase activity).

In some cases, a Cas polypeptide having a DNA binding domain can include one or more additional polypeptides (e.g., a subcellular localization signal such as a nuclear localization signal (NLS)).

In some cases, a Cas polypeptide having a DNA binding domain can be as described elsewhere (see, e.g., Cong et al., Science 339 (6121): 819-23 (2013); Hsu et al., Nat. Biotechnol., 31:827-832 (2013); Jinek et al., Science, 337 (6096): 816-21 (2012); Mali et al., Science, 339 (6121): 823-6 (2013); Nishimasu et al., Cell, 156 (5): 935-49 (2014); and Friedland et al., Genome Biol., 16:257 (2015)).

In cases where a polypeptide having a DNA binding domain includes a polymerase, the polymerase can be any appropriate polymerase. In some cases, the polymerase can be a transcriptase (e.g., reverse transcriptase). Examples of polymerases include, without limitation, reverse transcriptases from a Moloney murine leukemia virus (M-MLV RTs), reverse transcriptases from an avian myeloblastosis virus (AMV RTs), and reverse transcriptases from a human immunodeficiency virus type 1 (HIV-1 RTs). In some cases, a polymerase can be as described elsewhere (see, e.g., Gao et al., bioRxiv doi.org/10.1101/2021.11.05.467423 (2021)).

A polymerase (e.g., a reverse transcriptase) can have any appropriate amino acid sequence. Examples of polymerase sequences include, without limitation, amino acid sequences set forth in SEQ ID NO:7, SEQ ID NO:8, SEQ ID NO:9, or SEQ ID NO:10. In some cases, a polymerase can have one or more amino acid modifications (e.g., one or more insertions, one or more deletions, and/or one or more substitutions) relative to a polymerase described herein (e.g., SEQ ID NO:7, SEQ ID NO:8, SEQ ID NO:9, and SEQ ID NO:10), provided the polymerase maintains the ability to synthesize nucleic acid (e.g., maintains its polymerase activity). In some cases, a polymerase can have at least 70% (e.g., 70%, 75%, 80%, 85%, 90%, 93%, 95%, 96%, 97%, 98%, or 99%) sequence identity to any one of SEQ ID NO: 7, SEQ ID NO:8, SEQ ID NO:9, and SEQ ID NO: 10, provided the polymerase maintains the ability to synthesize nucleic acid (e.g., maintains its polymerase activity).

In some cases, a polymerase (e.g., a reverse transcriptase) can include one or more additional polypeptides (e.g., a subcellular localization signal such as a NLS).

In some cases, a polymerase (e.g., a reverse transcriptase) can be as described elsewhere (see, e.g., Baranauskas et al., Protein Eng. Des. Sel., 25 (10): 657-68 (2012); Anzalone et al., Nature, 576 (7785): 149-157 (2019); Ioannidi et al., BioRxiv, DOI 10.1101/2021.11.01.466786 (2021); Perbal et al., Retrovirology, 5:49 (2008); Konishi et al., Biotechnol. Lett., 34 (7): 1209-15 (2012); Hu et al., Cold Spring Harb. Perspect. Med., 2 (10): a006882 (2012); UniProt Accession No. Q9WJQ2; and Japanese Patent Application Publication JP2012120506A).

A nucleic acid molecule including a guide sequence that is complementary to a target site and a nucleic acid sequence that encodes an attA site in a genome editing system provided herein can include any appropriate guide sequence. In some cases, a guide sequence can be a guide RNA (gRNA). A guide sequence can be complementary to (e.g., can be designed to be complementary to) any appropriate target site. It will be appreciated that a target site within a genome can be designed specifically for the desired outcome of the stably integrated nucleic acid. For example, when a stably integrated nucleic acid is designed to express a transgene, the target site can be designed such that expression of any endogenous nucleic acid is not disrupted. For example, when a stably integrated nucleic acid is designed to disrupt and/or replace an endogenous nucleic acid encoding a polypeptide, the target site can be designed to be within the endogenous nucleic acid encoding the polypeptide (e.g., a coding sequence within that endogenous nucleic acid or a non-coding sequence within that endogenous nucleic acid).

A nucleic acid molecule including a guide sequence that is complementary to a target site and a nucleic acid sequence that encodes an attA site in a genome editing system provided herein can include any appropriate nucleic acid sequence that encodes an attA site. An attA site, as used herein, is an attachment site for an integrase described herein. In some cases, an attA site can be an acceptor attachment site derived from a bacterial target sequence (e.g., an attB site). In some cases, an attA site can be acceptor attachment site derived from a phage target sequence (e.g., an attP site).

In some cases, nucleic acid molecule including a guide sequence that is complementary to a target site and a nucleic acid sequence that encodes an attA site in a genome editing system provided herein can be engineered to include a nucleic acid sequence that encodes an attA site. For example, a nucleic acid sequence that encodes an attA site can be inserted into a nucleic acid using standard cloning or oligo capture techniques.

An attA site can be any appropriate length (e.g., can include any number of nucleotides). In some cases, an attA site can include from about 20 nucleotides to about 100 nucleotides (e.g., from about 20 nucleotides to about 90 nucleotides, from about 20 nucleotides to about 80 nucleotides, from about 20 nucleotides to about 70 nucleotides, from about 20 nucleotides to about 60 nucleotides, from about 20 nucleotides to about 50 nucleotides, from about 20 nucleotides to about 40 nucleotides, from about 20 nucleotides to about 30 nucleotides, from about 30 nucleotides to about 100 nucleotides, from about 40 nucleotides to about 100 nucleotides, from about 50 nucleotides to about 100 nucleotides, from about 60 nucleotides to about 100 nucleotides, from about 70 nucleotides to about 100 nucleotides, from about 80 nucleotides to about 100 nucleotides, from about 90 nucleotides to about 100 nucleotides, from about 30 nucleotides to about 90 nucleotides, from about 40 nucleotides to about 80 nucleotides, from about 50 nucleotides to about 70 nucleotides, from about 30 nucleotides to about 50 nucleotides, from about 40 nucleotides to about 60 nucleotides, from about 50 nucleotides to about 70 nucleotides, from about 60 nucleotides to about 80 nucleotides, or from about 70 nucleotides to about 90 nucleotides). For example, an attA site can include from about 25 nucleotides to about 45 nucleotides.

An attA site can include any appropriate nucleic acid sequence. Examples of attA sequences include, without limitation, nucleic acid sequences set forth in SEQ ID NOs: 11-84 and SEQ ID NO:254. In some cases, an attA site can have one or more amino acid modifications (e.g., one or more insertions, one or more deletions, and/or one or more substitutions) relative to an attA site described herein (e.g., SEQ ID NOs: 11-84 and SEQ ID NO: 254), provided the attA site maintains the ability to be recognized and recombined by an integrase (e.g., a LSR). In some cases, an attA site can have at least 50% (e.g., 50%, 55%, 60%, 65%, 70%, 75%, 80%, 85%, 90%, 93%, 95%, 96%, 97%, 98%, or 99%) sequence identity to a sequence set forth in any one of SEQ ID NOs: 11-84 and SEQ ID NO:254, provided that the attA site maintains the ability to be recognized and recombined by an integrase (e.g., a LSR).

In some cases, an attA sequence can be as described elsewhere (see, e.g., U.S. Ser. No. 63/275,288, filed on Nov. 3, 2021).

A system for stably integrating one or more nucleic acids into a target site within the genome of a cell provided herein can include any appropriate integrase. As used herein, the term “integrase” refers to a polypeptide that can recognize an attA site and an attD site and can meditate nucleic acid recombination between the attA site and the attD site. In some cases, an integrase can be a serine recombinase such as a large serine recombinase (LSR). In some cases, an integrase can be a landing pad integrase. In some cases, an integrase can be a genome-targeting integrase. In some cases, an integrase can be a multi-targeting integrase. In some cases, an integrase can be linked (e.g., covalently linked) to a polypeptide comprising a DNA binding domain and, optionally, a polymerase. For example, in some cases an integrase and a polypeptide comprising a DNA binding domain and, optionally, a polymerase can be provided together (e.g., as a fusion polypeptide comprising both the integrase and the polypeptide comprising a DNA binding domain and, optionally, a polymerase). In some cases when an integrase is linked to a polypeptide comprising a DNA binding domain and, optionally, a polymerase, the integrase can be linked directly to the polypeptide comprising a DNA binding domain and, optionally, a polymerase. In some cases when an integrase is linked to a polypeptide comprising a DNA binding domain and, optionally, a polymerase, the integrase can be linked to the polypeptide comprising a DNA binding domain and, optionally, a polymerase via a linker (e.g., a peptide linker).

In some cases, an integrase (e.g., serine recombinase such as a LSR) can include any appropriate amino acid sequence. For example, an integrase can have an amino acid sequence that includes one or more of the motifs set forth in SEQ ID NOs:233-245 (written in the common Prosite format). Examples of integrase sequences include, without limitation, amino acid sequences set forth in SEQ ID NOs:85-158. In some cases, an integrase can have one or more amino acid modifications (e.g., one or more insertions, one or more deletions, and/or one or more substitutions) relative to an integrase described herein (e.g., SEQ ID NOs: 85-158), provided the integrase maintains the ability to recognize and recombine an attA site and an attD site. In some cases, an integrase can have at least 70% (e.g., 70%, 75%, 80%, 85%, 90%, 93%, 95%, 96%, 97%, 98%, or 99%) sequence identity to a sequence set forth in any one of SEQ ID NOs:85-158, provided that the integrase site maintains the ability to recognize and recombine an attA site and an attD site.

In some cases, an integrase (e.g., serine recombinase such as a LSR) can be as described elsewhere (see, e.g., U.S. Ser. No. 63/275,288, filed on Nov. 3, 2021).

A donor nucleic acid molecule including a nucleic acid cargo and an attD site in a system for stably integrating one or more nucleic acids into a target site within the genome of a cell provided herein can be any appropriate donor nucleic acid molecule. In some cases, a donor nucleic acid molecule can be a linear nucleic acid molecule. In some cases, a donor nucleic acid molecule can be a circular nucleic acid molecule (e.g., a plasmid or a minicircle).

A donor nucleic acid molecule can be any appropriate size (e.g., can include any number of nucleotides). In some cases, a donor nucleic acid molecule is from about 0.25 kb (250 nucleotides (nt)) to about 30 kb (e.g., from about 0.5 kb to about 30 kb, from about 1 kb to about 30 kb, from about 2 kb to about 30 kb, from about 5 kb to about 30 kb, from about 7 kb to about 30 kb, from about 10 kb to about 30 kb, from about 12 kb to about 30 kb, from about 15 kb to about 30 kb, from about 18 kb to about 30 kb, from about 20 kb to about 30 kb, from about 22 kb to about 30 kb, from about 25 kb to about 30 kb, from about 27 kb to about 30 kb, from about 0.25 kb to about 30 kb, from about 0.5 kb to about 25 kb, from about 1 kb to about 20 kb, from about 2 kb to about 15 kb, from about 5 kb to about 10 kb, from about 0.25 kb to about 25 kb, from about 0.25 kb to about 20 kb, from about 0.25 kb to about 15 kb, from about 0.25 kb to about 10 kb, from about 0.25 kb to about 7 kb, from about 0.25 kb to about 5 kb, from about 0.25 kb to about 3 kb, from about 0.25 kb to about 1 kb, from about 0.25 kb to about 0.5 kb, from about 0.25 kb to about 0.75 kb, from about 1 kb to about 5 kb, from about 2 kb to about 4 kb, from about 3 kb to about 7 kb, from about 5 kb to about kb, from about 7 kb to about 12 kb, from about 12 kb to about 15 kb, from about 15 kb to about 18 kb, from about 18 kb to about 22 kb, from about 22 kb to about 25 kb, or from about 25 kb to about 28 kb). For example, a donor nucleic acid molecules can be from about 5 kb to about 30 kb.

A donor nucleic acid molecule can include any appropriate nucleic acid cargo. A nucleic acid cargo can be any polynucleotide sequence that can be delivered to and inserted into a target site within the genome of a cell using a system for stably integrating one or more nucleic acids into a target site within the genome of a cell provided herein. In some cases, a nucleic acid cargo can include a nucleic acid encodes a gene product (e.g., a polypeptide or a non-coding RNA). For example, a nucleic acid cargo in a donor nucleic acid molecule of a system for stably integrating one or more nucleic acids into a target site within the genome of a cell provided herein can encode a polypeptide. Examples of polypeptides that can be encoded by a nucleic acid cargo in a donor nucleic acid molecule include, without limitation, detectable labels (e.g., peptide tags, fluorescent polypeptides, and enzymes), therapeutic polypeptides and biologically active fragments thereof (e.g., polypeptides useful for treating a diseases and/or condition) such as transcription factors, genome engineering systems, and polypeptides for eliciting an immune response, antibodies. For example, a nucleic acid cargo in a donor nucleic acid molecule of a system for stably integrating one or more nucleic acids into a target site within the genome of a cell provided herein can encode a RNA (e.g., a non-coding RNA). Examples of RNA that can be encoded by a nucleic acid cargo in a donor nucleic acid molecule include, without limitation, tRNA, rRNA, inhibitory RNAs (e.g., antisense RNAs, microRNAs (miRNAs), small interfering RNAs (siRNAs), short hairpin RNAs (shRNAs), and agomiRs), antagomiRs, aptamers, and long non-coding RNAs (lncRNAs).

In cases where a donor nucleic acid molecule includes nucleic acid cargo that can encode a gene product, the donor nucleic acid also can include one or more regulatory elements operably linked to the nucleic acid encoding the gene product. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) can depend on several factors, including, without limitation, inducibility, targeting, and the level of expression desired. For example, a promoter can be included in a donor nucleic acid molecule to facilitate transcription of a nucleic acid cargo encoding a gene product. A promoter can be a naturally occurring promoter or a recombinant promoter. A promoter can be ubiquitous or inducible (e.g., in the presence of tetracycline), and can affect the expression of a nucleic acid encoding a gene product in a general or tissue-specific manner. Examples of promoters include, without limitation, human ubiquitin C promoters, human synapsin 1 gene promoters, human glial fibrillary acidic protein promoters, promoters with tetracycline response elements, human elongation factor-1 alpha promoters, cytomegalovirus promoters, CAG promoters, simian vacuolating virus 40 promoters, phosphoglycerate kinase gene promoters, and Ca²⁺/calmodulin-dependent protein kinase II promoters. As used herein, “operably linked” refers to positioning of a regulatory element in a donor nucleic acid molecule relative to a nucleic acid encoding a gene product in such a way as to permit or facilitate expression of the encoded gene product. For example, a donor nucleic acid molecule can contain a promoter and nucleic acid encoding a polypeptide. In this case, the promoter is operably linked to a nucleic acid encoding a polypeptide such that it drives expression of the polypeptide in cells. For example, a donor nucleic acid molecule can contain a promoter and nucleic acid encoding a non-coding RNA. In this case, the promoter is operably linked to a nucleic acid encoding a polypeptide such that it drives expression of the non-coding RNA in cells.

In some cases, a donor nucleic acid molecule can include one or more additional nucleic acid elements. For example, a donor nucleic acid molecule can be flanked by inverted terminal repeats (ITRs; e.g., AAV ITRs).

In some cases, a donor nucleic acid molecule can include an attD site and, optionally, nucleic acid cargo that can encode a gene product, and can lack any other nucleic acid elements. For example, when a donor nucleic acid molecule is a plasmid, bacterial elements such as an origin of replication (Ori) site can be removed from the plasmid. For example, when a donor nucleic acid molecule is a plasmid, other coding sequences such as nucleic acid encoding a selectable marker such as an antibiotic resistance gene can be removed from the plasmid.

A donor nucleic acid molecule can include any appropriate attD site. In some cases, an attD site can be donor attachment site derived from a phage donor sequence (e.g., an attP site).

An attD site can be any appropriate length (e.g., can include any number of nucleotides). In some cases, an attD site can include from about 20 nucleotides to about 100 nucleotides (e.g., from about 20 nucleotides to about 90 nucleotides, from about 20 nucleotides to about 80 nucleotides, from about 20 nucleotides to about 70 nucleotides, from about 20 nucleotides to about 60 nucleotides, from about 20 nucleotides to about 50 nucleotides, from about 20 nucleotides to about 40 nucleotides, from about 20 nucleotides to about 30 nucleotides, from about 30 nucleotides to about 100 nucleotides, from about 40 nucleotides to about 100 nucleotides, from about 50 nucleotides to about 100 nucleotides, from about 60 nucleotides to about 100 nucleotides, from about 70 nucleotides to about 100 nucleotides, from about 80 nucleotides to about 100 nucleotides, from about 90 nucleotides to about 100 nucleotides, from about 30 nucleotides to about 90 nucleotides, from about 40 nucleotides to about 80 nucleotides, from about 50 nucleotides to about 70 nucleotides, from about 30 nucleotides to about 50 nucleotides, from about 40 nucleotides to about 60 nucleotides, from about 50 nucleotides to about 70 nucleotides, from about 60 nucleotides to about 80 nucleotides, or from about 70 nucleotides to about 90 nucleotides). For example, an attD site can include from about 25 nucleotides to about 45 nucleotides.

An attD site can include any appropriate nucleic acid sequence. Examples of attD sequences include, without limitation, nucleic acid sequences set forth in SEQ ID NOs:159-232. In some cases, an attD site can have one or more amino acid modifications (e.g., one or more insertions, one or more deletions, and/or one or more substitutions) relative to an attD site described herein (e.g., SEQ ID NOs:159-232), provided the attD site maintains the ability to be recognized and recombined by an integrase (e.g., an LSR). In some cases, an attD site can have at least 50% (e.g., 50%, 55%, 60%, 65%, 70%, 75%, 80%, 85%, 90%, 93%, 95%, 96%, 97%, 98%, or 99%) sequence identity to a sequence set forth in any one of SEQ ID NOs: 159-232, provided that the attD site maintains the ability to be recognized and recombined by an integrase (e.g., a LSR).

In some cases, an attD sequence can be as described elsewhere (see, e.g., U.S. Ser. No. 63/275,288, filed on Nov. 3, 2021).

Also provided herein are methods for using systems for stably integrating one or more nucleic acids into a target site within the genome of a cell provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site). In some cases, a system for stably integrating one or more nucleic acids into a target site within the genome of a cell provided herein can be delivered to a cell to stably integrate a nucleic acid into the genome of the cell. For example, a system including (a) a genome-editing system that can insert an attA into a target site within a genome, (b) a donor nucleic acid molecule including a nucleic acid cargo and an attD site, and (c) an integrase (e.g., a LSR) that can target the attA site and the attD site can be delivered to a cell to stably integrate the nucleic acid cargo into the genome of the cell. In some cases, the components of a system for stably integrating one or more nucleic acids into a target site within the genome of a cell provided herein can be delivered to a cell in vitro. In some cases, the components of a system for stably integrating one or more nucleic acids into a target site within the genome of a cell provided herein can be delivered to a cell ex vivo. In some cases, the components of a system for stably integrating one or more nucleic acids into a target site within the genome of a cell provided herein can be delivered to a cell in vivo.

Any appropriate method can be used to deliver components of a system for stably integrating one or more nucleic acids into a target site within the genome of a cell provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) to cells (e.g., cells within a living mammal). In some cases, a genome-editing system that can insert an attA into a target site within a genome can be delivered to a cell as a complex including (i) a polypeptide having a DNA binding domain and, optionally, a polymerase and (ii) a nucleic acid molecule including a guide sequence that is complementary to the target site and a nucleic acid sequence that encodes an attA site. In some cases, a genome-editing system that can insert an attA into a target site within a genome can be delivered to a cell as a nucleic acid encoding the genome-editing system (e.g., a vector designed to express the genome-editing system) such that a complex including (i) a polypeptide having a DNA binding domain and, optionally, a polymerase and (ii) a nucleic acid molecule including a guide sequence that is complementary to the target site and a nucleic acid sequence that encodes an attA site is formed within the cell. In some cases, an integrase that can target the attA site and the attD site can be delivered to a cell as a polypeptide. In some cases, an integrase that can target the attA site and the attD site can be delivered to a cell as a nucleic acid encoding the integrase (e.g., a vector designed to express the integrase). In some cases, a donor nucleic acid molecule including a nucleic acid cargo and an attD site can be delivered to a cell as a linear nucleic acid molecule. In some cases, a donor nucleic acid molecule including a nucleic acid cargo and an attD site can be delivered to a cell as a circular nucleic acid (e.g., a vector). For example, a genome-editing system that can insert an attA into a target site within a genome and an integrase that can target the attA site and the attD site can be delivered to a cell as polypeptides, and a donor nucleic acid molecule including a nucleic acid cargo and an attD site are administered to cell can be delivered to the cell in the form of a vector (e.g., a non-viral vector). In some cases, nucleic acid encoding a genome-editing system that can insert an attA into a target site within a genome, nucleic acid encoding an integrase that can target the attA site and the attD site, and a donor nucleic acid molecule including a nucleic acid cargo and an attD site can be delivered to a cell in the form of one or more vectors (e.g., one or more viral vectors and/or one or more non-viral vectors).

When a vector used to deliver nucleic acid encoding a genome-editing system that can insert an attA into a target site within a genome, nucleic acid encoding an integrase that can target the attA site and the attD site, and/or a donor nucleic acid molecule including a nucleic acid cargo and an attD site is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. Examples of virus-based vectors that can be used to deliver nucleic acid encoding a genome-editing system that can insert an attA into a target site within a genome, nucleic acid encoding an integrase that can target the attA site and the attD site, and/or a donor nucleic acid molecule including a nucleic acid cargo and an attD site include, without limitation, virus-based vectors based on adenoviruses, adeno-associated viruses (AAVs), Sendai viruses, retroviruses, or lentiviruses. In some cases, a donor nucleic acid molecule including a nucleic acid cargo and an attD site can be delivered on an AAV.

When a vector used to deliver nucleic acid encoding a genome-editing system that can insert an attA into a target site within a genome, nucleic acid encoding an integrase that can target the attA site and the attD site, and/or a donor nucleic acid molecule including a nucleic acid cargo and an attD site is a non-viral vector, any appropriate non-viral vector can be used. In some cases, a non-viral vector can be an expression plasmid (e.g., a cDNA expression vector).

When nucleic acid encoding a genome-editing system that can insert an attA into a target site within a genome and/or nucleic acid encoding an integrase is delivered to a cell, the nucleic acid can be used for transient expression of a genome-editing system and/or an integrase or for stable expression of a genome-editing system and/or an integrase.

In cases where a nucleic acid encoding a genome-editing system that can insert an attA into a target site within a genome and/or nucleic acid encoding an integrase is used to deliver a genome-editing system and/or an integrase to a cell, the nucleic acid also can include one or more regulatory elements operably linked to the nucleic acid encoding the genome-editing system and/or the integrase. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) can depend on several factors, including, without limitation, inducibility, targeting, and the level of expression desired. For example, a promoter can be included in a nucleic acid encoding a genome-editing system that can insert an attA into a target site within a genome and/or nucleic acid encoding an integrase to facilitate transcription of the genome-editing system and/or the integrase. A promoter can be a naturally occurring promoter or a recombinant promoter. A promoter can be ubiquitous or inducible (e.g., in the presence of tetracycline), and can affect the expression of a nucleic acid encoding a gene product in a general or tissue-specific manner. Examples of promoters include, without limitation, human ubiquitin C promoters, human synapsin 1 gene promoters, human glial fibrillary acidic protein promoters, promoters with tetracycline response elements, human elongation factor-1 alpha promoters, cytomegalovirus promoters, CAG promoters, simian vacuolating virus 40 promoters, phosphoglycerate kinase gene promoters, and Ca²⁺/calmodulin-dependent protein kinase II promoters. As used herein, “operably linked” refers to positioning of a regulatory element in a donor nucleic acid molecule relative to a nucleic acid encoding a genome-editing system that can insert an attA into a target site within a genome and/or nucleic acid encoding an integrase in such a way as to permit or facilitate expression of the encoded genome-editing system and/or the encoded integrase. For example, a nucleic acid encoding a genome-editing system that can insert an attA into a target site within a genome can contain a promoter and nucleic acid encoding a genome-editing system. In this case, the promoter is operably linked to a nucleic acid encoding a genome-editing system that can insert an attA into a target site within a genome such that it drives expression of the genome-editing system in cells. For example, a nucleic acid encoding an integrase can contain a promoter and nucleic acid encoding the integrase. In this case, the promoter is operably linked to a nucleic acid encoding an integrase such that it drives expression of the integrase in cells.

In some cases, the components of a system for stably integrating one or more nucleic acids into a target site within the genome of a cell provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be delivered to cells (e.g., cells within a living mammal) at the same time. For example, a system for stably integrating one or more nucleic acids into a target site within the genome of a cell can be delivered to a cell in a single composition containing (a) a genome-editing system that can insert an attA into a target site within a genome (or nucleic acid encoding such a genome-editing system), (b) a donor nucleic acid molecule including a nucleic acid cargo and an attD site, and (c) an integrase (e.g., a LSR) that can target the attA site and the attD site (or nucleic acid encoding such an integrase). For example, a system for stably integrating one or more nucleic acids into a target site within the genome of a cell can be delivered to a cell in a single composition containing (a) a genome-editing system that can insert an attA into a target site within a genome linked (e.g., covalently linked as a fusion polypeptide) to (b) a donor nucleic acid molecule including a nucleic acid cargo and an attD site, and containing (c) an integrase (e.g., a LSR) that can target the attA site and the attD site. For example, a system for stably integrating one or more nucleic acids into a target site within the genome of a cell can be delivered to a cell in a single composition containing a nucleic acid encoding a polypeptide (e.g., a fusion polypeptide) including both a genome-editing system that can insert an attA into a target site within a genome linked and an integrase (e.g., a LSR) that can target the attA site and an attD site, and a donor nucleic acid molecule including a nucleic acid cargo and the attD site.

In some cases, the components of a system for stably integrating one or more nucleic acids into a target site within the genome of a cell provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be delivered to cells (e.g., cells within a living mammal) independently. For example, a system for stably integrating one or more nucleic acids into a target site within the genome of a cell can be delivered to a cell as in a first composition containing (a) a genome-editing system that can insert an attA into a target site within a genome (or nucleic acid encoding such a genome-editing system), and a second composition containing (b) a donor nucleic acid molecule including a nucleic acid cargo and an attD site and (c) an integrase (e.g., a LSR) that can target the attA site and the attD site (or nucleic acid encoding such an integrase). For example, a system for stably integrating one or more nucleic acids into a target site within the genome of a cell can be delivered to a cell as in a first composition containing (a) a genome-editing system that can insert an attA into a target site within a genome (or nucleic acid encoding such a genome-editing system) and (b) a donor nucleic acid molecule including a nucleic acid cargo and an attD site, and a second composition containing (c) an integrase (e.g., a LSR) that can target the attA site and the attD site (or nucleic acid encoding such an integrase). For example, a system for stably integrating one or more nucleic acids into a target site within the genome of a cell can be delivered to a cell as in a first composition containing (a) a genome-editing system that can insert an attA into a target site within a genome (or nucleic acid encoding such a genome-editing system), a second composition containing (b) a donor nucleic acid molecule including a nucleic acid cargo and an attD site, and a third composition containing (c) an integrase (e.g., a LSR) that can target the attA site and the attD site (or nucleic acid encoding such an integrase).

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used for labelling a gene product (e.g., a polypeptide or a non-coding RNA) within a cell (e.g., a plant cell or a mammalian cell). For example, the methods and materials provided herein can be used to label a gene product encoded by an endogenous nucleic acid within a cell (e.g., a prokaryotic cell or a eukaryotic cell such as a plant cell or an animal cell). In some cases, a gene product within a cell can be labeled by delivering a system for stably integrating one or more nucleic acids into a target site within the genome of a cell provided herein (e.g., a system including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) to a cell (e.g., a plant cell or a mammalian cell) to stably integrate a nucleic acid encoding a detectable label in-frame with an endogenous nucleic acid encoding a target gene product such that the encoded target gene product is fused to the detectable label. For example, (a) a genome-editing system that can insert an attA into a target site within a genome that is in-frame with an endogenous nucleic acid encoding a target gene product, (b) a donor nucleic acid molecule including a nucleic acid cargo encoding a detectable label and an attD site, and (c) an integrase that can target the attA site and the attD site can be delivered to a cell to stably integrate the nucleic acid cargo encoding the detectable label into the genome such that the encoded target gene product is fused to the detectable label.

When a nucleic acid cargo encoding a detectable label is stably integrated into the genome of a cell (e.g., a plant cell or a mammalian cell) to label a target polypeptide within the cell, any appropriate detectable label can be used. Examples of detectable labels include, without limitation, luminescent tags (e.g., HiBiT), peptide tags (e.g., HaloTag, Flag tags, HA tags, MS2/PP7 tags, Sun/Moon tags, and poly(His) tags), fluorescent polypeptides (e.g., mCherry and green fluorescent polypeptides (GFPs; e.g., mNeonGreen)), and enzymes (e.g., glutathione-S-transferases (GSTs), luciferases, horseradish peroxidases (HRPs), alkaline phosphatases (APs), and apurinic/apyrimidinic endodeoxyribonuclease 2 (APEX2) polypeptides).

In some cases, a nucleic acid cargo encoding a detectable label can be integrated into the genome upstream of an endogenous nucleic acid encoding a target polypeptide such that the detectable label is fused to the N-terminus of the target polypeptide.

In some cases, a nucleic acid cargo encoding a detectable label can be integrated into the genome downstream of an endogenous nucleic acid encoding a target polypeptide such that the detectable label is fused to the C-terminus of the target polypeptide.

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used to increase expression of a polypeptide within a cell (e.g., a plant cell or a mammalian cell). For example, the methods and materials provided herein can be used to increase expression of a polypeptide encoded by an endogenous nucleic acid within a cell (e.g., a prokaryotic cell or a eukaryotic cell such as a plant cell or an animal cell). In some cases, expression of a polypeptide within a cell can be increased by delivering a system for stably integrating one or more nucleic acids into a target site within the genome of a cell provided herein (e.g., a system including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) to a cell (a plant cell or a mammalian cell) to stably integrate a regulatory element (e.g., a promoter sequence) near (e.g., upstream of) an endogenous nucleic acid encoding a target polypeptide such that the regulatory element is operably linked to and increases expression of the encoded target polypeptide. For example, (a) a genome-editing system that can insert an attA into a target site within a genome near an endogenous nucleic acid encoding a target polypeptide, (b) a donor nucleic acid molecule including a nucleic acid cargo containing a promoter sequence and an attD site, and (c) an integrase that can target the attA site and the attD site can be delivered to a cell to stably integrate the promoter sequence into the genome such that the expression of the encoded target polypeptide is increased.

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used for making a transgenic organism (e.g., a non-human transgenic organism). For example, the methods and materials provided herein can be used to express an exogenous polypeptide within a cell such as a eukaryotic cell. In some cases, the methods and materials provided herein can be used to stably integrate a transgene (e.g., a transgene encoding an exogenous polypeptide) into the genome of a cell (e.g., an embryonic stem cell) that can give rise to an animal (e.g., a non-human animal). In some cases, the methods and materials provided herein can be used to stably integrate a transgene (e.g., a transgene encoding an exogenous polypeptide) into the genome of a cell (e.g., a plant cell) that can give rise to a plant.

In some cases, a transgenic organism (e.g., a non-human transgenic organism) can be created by delivering a system for stably integrating one or more nucleic acids into a target site within the genome of a cell provided herein (e.g., a system including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) to a cell (e.g., a plant cell or a non-human embryonic stem cell) to stably integrate a transgene (e.g., a transgene encoding a polypeptide of interest) into the genome such that the transgene is expressed by the cell. For example, (a) a genome-editing system that can insert an attA into a target site within a genome, (b) a donor nucleic acid molecule including a transgene and an attD site, and (c) an integrase that can target the attA site and the attD site can be delivered to a cell to stably integrate the transgene into the genome such that the transgene is expressed by the cell.

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used for making a transgenic cell (e.g., a transgenic immune cell such as a transgenic T cell, a transgenic NK cell, or a transgenic macrophage) having (e.g., engineered to have) a receptor (e.g., a T cell receptor (TCR); a NK cell receptor (NKR), or a chimeric antigen receptor (CAR)). For example, (a) a genome-editing system that can insert an attA into a target site within a genome, (b) a donor nucleic acid molecule including a transgene encoding a CAR and an attD site, and (c) an integrase that can target the attA site and the attD site can be delivered to a T cell (e.g., an ex vivo human T cell) to stably integrate the transgene into the genome of the T cell such that the CAR is expressed by the T cell (e.g., to generate a CAR T cell). For example, (a) a genome-editing system that can insert an attA into a target site within a genome, (b) a donor nucleic acid molecule including a transgene encoding a TCR (e.g., a wild type TCR or an engineered TCR) and an attD site, and (c) an integrase that can target the attA site and the attD site can be delivered to an NK cell (e.g., an ex vivo human NK cell) to stably integrate the transgene into the genome of the NK cell such that the TCR is expressed by the NK cell (e.g., to generate an NK cell expressing the TCR). For example, (a) a genome-editing system that can insert an attA into a target site within a genome, (b) a donor nucleic acid molecule including a transgene encoding a NKR (e.g., a wild type NKR or an engineered NKR) and an attD site, and (c) an integrase that can target the attA site and the attD site can be delivered to an NK cell (e.g., an ex vivo human NK cell) to stably integrate the transgene into the genome of the NK cell such that the NKR is expressed by the NK cell (e.g., to generate an NK cell expressing the NKR). Any appropriate receptor (e.g., any appropriate TCR, any appropriate NKR, or any appropriate CAR) can be integrated into the genome of a cell (e.g., an immune cell such as a T cell or a NK cell) as described herein. In some cases, a CAR can be as described elsewhere (e.g., De Bousser et al., Cancers (Basel), 13 (23): 6067 (2021); Eyquem et al., Nature, 543 (7643): 113-117 (2017); and Larson et al., Nat. Rev. Cancer, 21 (3): 145-161 (2021)).

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used for making a transgenic plant having (e.g., engineered to have) pathogen resistance (e.g., bacterial resistance or viral resistance). For example, (a) a genome-editing system that can insert an attA into a target site within a genome, (b) a donor nucleic acid molecule including a transgene encoding a pathogen resistance polypeptide and an attD site, and (c) an integrase that can target the attA site and the attD site can be delivered to a plant cell to stably integrate the transgene into the genome such that the pathogen resistance polypeptide is expressed by the cell. Any appropriate pathogen resistance polypeptide can be integrated into a plant cell genome to create a pathogen resistant transgenic plant as described herein. In some cases, a pathogen resistance polypeptide can be as described elsewhere (e.g., Dong et al., Plant Physiol., 180 (1): 26-38 (2019)).

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used for making a transgenic plant having (e.g., engineered to have) herbicide resistance. For example, (a) a genome-editing system that can insert an attA into a target site within a genome, (b) a donor nucleic acid molecule including a transgene encoding a herbicide resistance polypeptide and an attD site, and (c) an integrase that can target the attA site and the attD site can be delivered to a plant cell to stably integrate the transgene into the genome such that the herbicide resistance polypeptide is expressed by the cell. Any appropriate herbicide resistance polypeptide can be integrated into a plant cell genome to create an herbicide resistant transgenic plant as described herein. In some cases, an herbicide resistance polypeptide can be as described elsewhere (e.g., Sun et al., Molecular Plant, 9.4:628-631 (2016); Li et al., Nature Plants, 2:16139 (2016); Tatsis et al., Curr. Opin. Biotech., 42:126-132 (2016); Ducat et al., Curr. Opin. Chem. Biol., 16 (3-4): 337-344 (2012); Sanghera et al., Curr. Genomics., 12 (1): 30-43 (2011); Dong et al., Nat. Commun., 11:1178 (2020); and Lu et al., Nat. Biotechnol., 38:1402-1407 (2020)).

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used for making an organism (e.g., a non-human organism) having reduced or eliminated levels of a polypeptide (e.g., a non-human knock-out organism). For example, the methods and materials provided herein can be used to disrupt and/or replace an endogenous nucleic acid encoding a target polypeptide within a cell such as a eukaryotic cell. In some cases, the methods and materials provided herein can be used to stably integrate a nucleic acid molecule (e.g., knock-out cassette) into the genome of a cell (e.g., an embryonic stem cell) that can give rise to an organism (e.g., a non-human animal) to disrupt and/or replace an endogenous nucleic acid encoding a target polypeptide. In some cases, the methods and materials provided herein can be used to stably integrate a nucleic acid molecule (e.g., knock-out cassette) into the genome of a cell (e.g., a plant cell) that can give rise to a plant to disrupt and/or replace an endogenous nucleic acid encoding a target polypeptide.

In some cases, an endogenous nucleic acid encoding a target polypeptide within a cell can be disrupted and/or replaced by delivering a system for stably integrating one or more nucleic acids into a target site within the genome of a cell provided herein (e.g., a system including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) to a cell (a plant cell or a mammalian cell) to stably integrate a nucleic acid molecule within an endogenous nucleic acid encoding a target polypeptide such that the nucleic acid molecule disrupts and/or replaces the endogenous nucleic acid encoding a target polypeptide and expression of the endogenous nucleic acid encoding the target polypeptide is reduced or eliminated. For example, (a) a genome-editing system that can insert an attA into a target site within a genome that is in-frame with an endogenous nucleic acid encoding a target polypeptide, (b) a donor nucleic acid molecule including a nucleic acid cargo and an attD site, and (c) an integrase that can target the attA site and the attD site can be delivered to a cell to stably integrate the nucleic acid cargo into the genome such that the nucleic acid cargo disrupts and/or replaces an endogenous nucleic acid encoding a target polypeptide such that the nucleic acid molecule disrupts and/or replaces the endogenous nucleic acid encoding a target polypeptide and expression of the encoded target polypeptide is reduced or eliminated.

In some cases, a nucleic acid cargo that can be stably integrated into a genome of a cell (e.g., a non-human animal cell or a plant cell) to disrupt and/or replace an endogenous nucleic acid encoding a target polypeptide such that expression of the encoded the target polypeptide is reduced or eliminated can include a stop codon.

In some cases, a nucleic acid cargo that can be stably integrated into a genome of a cell (e.g., a non-human animal cell or a plant cell) to disrupt and/or replace an endogenous nucleic acid encoding a target polypeptide such that expression of the encoded the target polypeptide is reduced or eliminated can include a splice acceptor site.

In some cases, a nucleic acid cargo that can be stably integrated into a genome of a cell (e.g., a non-human animal cell or a plant cell) to disrupt and/or replace an endogenous nucleic acid encoding a target polypeptide such that expression of the encoded the target polypeptide is reduced or eliminated can include nucleic acid encoding a selectable marker such that the selectable marker is expressed by the cell. For example, a nucleic acid cargo can be stably integrated into a genome of a cell such that the selectable marker is under the control of the regulatory elements for the disrupted and/or replaced endogenous nucleic acid encoding a target polypeptide.

In some cases, a nucleic acid cargo that can be stably integrated into a genome of a cell (e.g., a non-human animal cell or a plant cell) to disrupt and/or replace an endogenous nucleic acid encoding a target polypeptide such that expression of the encoded the target polypeptide is reduced or eliminated can include a detectable label such that the detectable label is expressed by the cell. For example, a nucleic acid cargo can be stably integrated into a genome of a cell such that the detectable label is under the control of the regulatory elements for the disrupted and/or replaced endogenous nucleic acid encoding a target polypeptide.

In some cases, the methods and materials provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be used for treating a mammal (e.g., a human) having a disease or disorder. For example, (a) a genome-editing system that can insert an attA into a target site within a genome, (b) a donor nucleic acid molecule including a transgene encoding a therapeutic gene product and an attD site, and (c) an integrase that can target the attA site and the attD site can be delivered to a cell to stably integrate the transgene into the genome such that the therapeutic gene product is expressed by the cell. In some cases, the methods and materials provided herein can be used to treat a mammal (e.g., a human) have a disease or disorder associated with reduced or eliminated levels of a gene product (e.g., reduced or eliminated levels of a polypeptide or reduced or eliminated levels of a non-coding RNA). In some cases, the methods and materials provided herein can be used to treat a mammal (e.g., a human) have a disease or disorder associated with a mutated gene product (e.g., a mutated polypeptide or a mutated non-coding RNA).

When the methods and materials provided herein are used to treat a mammal, the mammal can be any appropriate mammal. Examples of mammals that can be treated as described herein include, without limitation, humans, non-human primates such as chimpanzees and monkeys, dogs, cats, horses, cows, pigs, sheep, mice, rats, rabbits, guinea pigs, birds, fish, (e.g., zebrafish (Danio rerio), medaka (Oryzias latipes), and turquoise killifish (Nothobranchius furzeri)), nematodes (e.g., Caenorhabditis elegans), and flies (e.g., Drosophila melanogaster).

In some cases when treating a mammal as described herein, the components of a system for stably integrating one or more nucleic acids into a target site within the genome of a cell provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be delivered to cells within a living mammal (e.g., can be delivered to in vivo cells).

In some cases when treating a mammal as described herein, the components of a system for stably integrating one or more nucleic acids into a target site within the genome of a cell provided herein (e.g., systems including (a) a genome-editing system that can insert an attA into a target site within a genome, (b) a donor nucleic acid molecule including a nucleic acid cargo and an attD site, and (c) an integrase (e.g., a LSR) that can target the attA site and the attD site) can be delivered to cells obtained from a mammal (e.g., can be delivered to ex vivo cells), and then the cells containing the stably integrated nucleic acid can be administered to the mammal to be treated. In some cases, the components of a system for stably integrating one or more nucleic acids into a target site within the genome of a cell provided herein are delivered ex vivo to cell obtained from the mammal to be treated (e.g., an autologous cell). In some cases, the components of a system for stably integrating one or more nucleic acids into a target site within the genome of a cell provided herein are delivered ex vivo to cell obtained from a donor mammal (e.g., an allogeneic cell).

Any appropriate transgene encoding a therapeutic gene product can be integrated into a cell genome to treat a mammal as described herein. Examples of therapeutic gene products include, without limitation, adenosine deaminase (e.g., to treat a mammal having severe combined immunodeficiency (SCID)), α-1 antitrypsin (e.g., to treat a mammal having liver damage such as cirrhosis), cystic fibrosis transmembrane conductance regulator (CFTR; e.g., to treat a mammal having cystic fibrosis (CF)), β-hemoglobin (HBB; e.g., to treat a mammal having thalassemia), oculocutaneous albinism II (OCA2; e.g., to treat a mammal having oculocutaneous albinism (OCA), Huntingtin (HTT; e.g., to treat a mammal having Huntington's disease), dystrophia myotonica-protein kinase (DMPK; e.g., to treat a mammal having myotonic dystrophy 1 (DM1)), low-density lipoprotein receptor (LDLR; e.g., to treat a mammal having familial hypercholesterolemia (FH)), apolipoprotein B (APOB; e.g., to treat a mammal having FH), neurofibromin 1 (NF1; e.g., to treat a mammal having neurofibromatosis), polycystic kidney disease 1 (PKD1; e.g., to treat a mammal having polycystic kidney disease), polycystic kidney disease 2 (PKD2; e.g., to treat a mammal having polycystic kidney disease), coagulation factor VIII (F8; e.g., to treat a mammal having hemophilia), dystrophin (DMD; e.g., to treat a mammal having Duchenne muscular dystrophy (DMD)), phosphate-regulating endopeptidase homologue X-linked (PHEX; e.g., to treat a mammal having hypophosphatemic rickets), methyl-CpG-binding protein 2 (MECP2; e.g., to treat a mammal having Rett Syndrome), ubiquitin-specific peptidase 9Y, Y-linked (USP9Y; e.g., to treat a mammal having spermatogenic failure), a carbamoyl-phosphate synthase 1 (CPS1) polypeptide, an ATP binding cassette subfamily A member 4 (ABCA4) polypeptide, an fatty acid elongase 4 (ELOVL) polypeptide, amyosin VIIA (MY07A) polypeptide, an usher syndrome 1C (USH1C) polypeptide, a cadherin related 23 (CDH23) polypeptide, a protocadherin related 15 (PCDH15) polypeptide, an usher syndrome 1G (USH1G) polypeptide, an usher syndrome 2A (USH2A) polypeptide, an adhesion G protein-coupled receptor V1 (ADGRV1) polypeptide, a whirlin (WHRN) polypeptide, a clarin 1 (CLRN1) polypeptide, a retinitis pigmentosa 1 (RP1) polypeptide, an eyes shut homolog (EYS) polypeptide, a lipoprotein (a) (LPA) polypeptide, a lipoprotein lipase (LPL) polypeptide, an apolipoprotein C2 (APOC2) polypeptide, an apolipoprotein A5 (APOA5) polypeptide, a lipase maturation factor 1 (LMF1) polypeptide, a glycosylphosphatidylinositol anchored high density lipoprotein binding protein 1 (GPIHBP1) polypeptide, a proprotein convertase subtilisin/kexin type 9 (PCSK9) polypeptide, a ryanodine receptor 2 (RYR2) polypeptide, a calsequestrin 2 (CASQ2) polypeptide, a myosin heavy chain 7 (MYH7) polypeptide, a myosin binding protein C3 (MYBPC3) polypeptide, a troponin T2, cardiac type (TNNT2) polypeptide, and a troponin 13, cardiac type (TNNI3) polypeptide, and C9orf72 polypeptide (e.g., to treat a mammal having C9orf72 amyotrophic lateral sclerosis and frontotemporal dementia (C9 ALS/FTD)). In some cases, a therapeutic gene product can be as described elsewhere (e.g., Suzuki et al., Mol. Ther., 28.7:1684-1695 (2020); Pierce et al., Cold Spring Harbor Perspect. Med. 5:9 a017285 (2015); Urnov et al., Nature, 435.7042:646-651 (2005); Phelps et al., Human Mol. Gen., 4.8:1251-1258 (1995); and Ellerby et al., Neurotherapeutics, 16 (4): 924-927 (2019)).

The invention will be further described in the following examples, which do not limit the scope of the invention described in the claims.

### EXAMPLES

### Example 1: Stable Integration of Multi-Kilobase DNA Cargos Into Eukaryotic Cell Genomes

Large serine recombinases (LSRs) are a family of enzymes encoded in phage genomes that site-specifically and unidirectionally recombine short DNA attachment sites present on phage and bacterial genome, resulting in integration of the multi-kilobase phage genome into the bacterial genome.

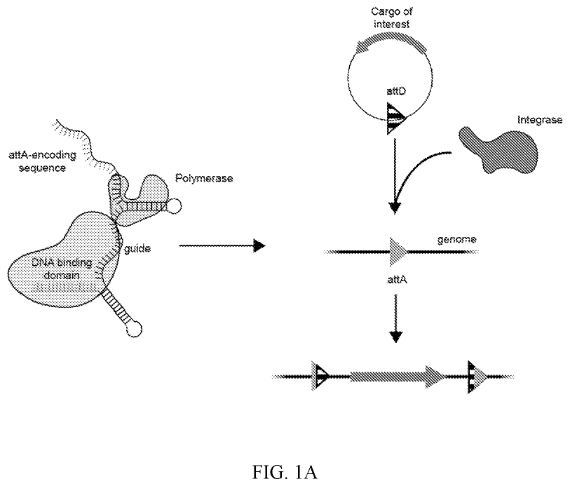

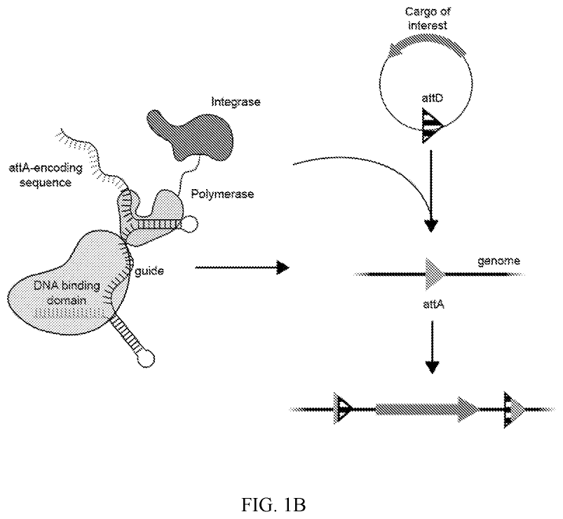

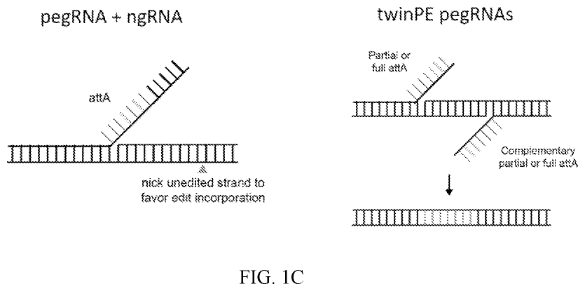

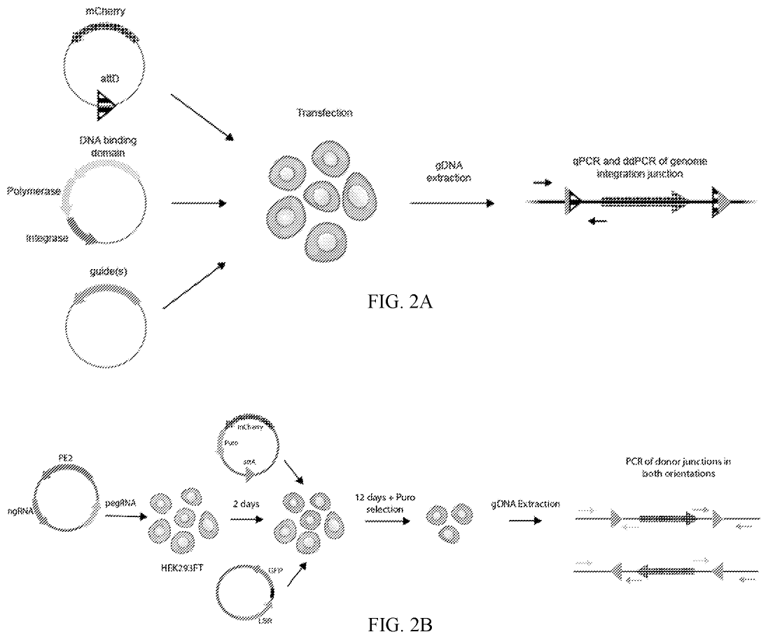

This Example describes the utilization of a prime editor in combination with a LSR for programmable recombination of multiple kilobase cargo into the genome. For example, a prime editor can be used to insert an attA site into a desired genomic context, and a LSR can integrate a nucleic acid cargo into the target site. Schematic images of exemplary methods of using a prime editor in combination with a LSR for programmable recombination of multiple kilobase cargo into the genome are shown in FIG. 1.

### Methods

Cloning of pegRNAs and ngRNAs

For pegRNAs, spacer sequences, extension templates, and SpCas9 sgRNA scaffold sequences were synthesized (Integrated DNA Technologies) and cloned via ligation of annealed oligonucleotides into BsmBI digested acceptor vector (pU6-pegRNA-GG-acceptor, Addgene plasmid no. 132777). For ngRNAs, spacers were synthesized (Integrated DNA Technologies) and cloned via ligation of annealed oligonucleotides into BbsI digested acceptor vector (pCB007 SpCas9_sgRNA_cloning_Backbone).

### Cell Lines and Cell Culture

Experiments were carried out in HEK-293 FT cells (Thermo Fisher). HEK-293 FT cells were grown in DMEM (Gibco) media supplemented with 10% FBS (Hyclone), penicillin (10,000 I.U./mL), and streptomycin (10,000 ug/mL).

### Prime Editing Transfection

20,000 HEK293FT cells were plated into poly-D-lysine coated 96 well plates. One day later, 250 ng prime editor plasmid (pCMV-PE2-P2A-GFP Addgene plasmid #132776), 83 ng pegRNA plasmid, and 27.6 ng ngRNA plasmid were transfected into the cells using Lipofectamine 2000 (Thermo). 3 days later, cells were extracted with DNA QuickExtract (Lucigen). Edits were verified via PCR (Platinum Superfi PCR Master Mix, Thermo) across the edited locus. Sanger sequencing was analyzed with ICE analysis (Synthego) to determine the percentage of cells containing the edit.

### 2-Step Transfection

Trans delivery. Prime editor, LSR and guide RNAs were transfected into HEK293FT cells in a single step or two step transfection. For two-step transfections, 20,000 HEK293FT cells were plated into poly-D-lysine coated 96 well plates. One day later, 250 ng prime editor plasmid, 83 ng pegRNA, and 27.6 ng ngRNA were transfected into the cells using Lipofectamine 2000 (Thermo). Two days later, 200 ng LSR effector plasmid and 100 ng attD donor plasmid were transfected into the cells using Lipofectamine 2000 (Thermo). Cells were harvested two days later using DNA QuickExtract (Lucigen). Prime editing and LSR mediated donor integration were confirmed using PCR (Platinum Superfi PCR Master Mix, Thermo Fisher) across the insertion junction. For one-step transfections, the same quantities of Prime editor, ngRNA, pegRNA, LSR, and donor plasmid were co-transfected on day 0, and cells were harvested on day 5 for PCR.

Sanger sequencing validation of donor integration. The Prime editing elements are transfected, and two days later the LSR and donor DNA are delivered. 4 days post-transfection, the gDNA is extracted, purified, and PCR and Sanger sequencing is performed across the donor-genome junction.

### Cloning PE-LSR Effector Plasmid

Prime editing plasmid (pCMV-PE2, Addgene Plasmid #132775) was modified with gibson cloning to include an XTEN 48 linker, a L139P mutation in the MMuLV RT, and either a (GGS) 6 (for cis LSR delivery) or a self-cleavable P2A (for trans LSR delivery) linker and BsmbI golden gate landing pad at the C terminus of the RT. Human codon optimized LSRs were cloned into the BsmBI landing pad via golden gate assembly.

### 1-Step Transfection and Integration Detection

Three plasmids containing the effector, donor, and guides are co-transfected into mammalian cells (HEK293FT). Three days later, gDNA is extracted, purified, and donor integration is determined by qPCR and ddPCR of the donor-genome junction.

1-Step Prime Editing, 1 pegRNA

20,000 HEK293FT cells were plated into poly-D-lysine coated 96 well plates. One day later, 375 ng effector plasmid, 100 ng pegRNA, and 50 ng ngRNA were transfected into the cells using Lipofectamine 2000 (Thermo). After 72 hours, media was removed and cells were resuspended in 40 uL DNA QuickExtract (Lucigen). Next, the cells were transferred to a PCR plate, and incubated at 65° C. for 15 minutes, 68° C. for 15 minutes, and 98° C. for 10 minutes. Finally, samples were purified with 0.9× Ampure XP beads (Beckman Coulter).

1-Step Prime Editing, 2 pegRNAs

Cells were plated as previously described and transfected with lipofectamine 2000, delivering 375 ng effector plasmid, 60 ng of each twinPE pegRNA, and 250 ng cargo plasmid. 72 hrs post transfection, cells were harvested and purified with DNA Quick Extract and Ampure XP beads.

qPCR Verification of Targeted Recombination.

qPCR primers and a FAM probe (IDT and Elim Bio) were designed to amplify the integration junction. As a genomic DNA reference, qPCR primers and a HEX probe (IDT and Elim Bio) were designed to amplify a non-edited region of the ACTB gene. 10 uL qPCR reactions were performed with 5 uL Taqman Fast Advanced 2× Master Mix, 250 nM of each primer, 200 nM of each probe, and 1 uL of extracted genomic DNA. qPCR was run on the 480 LightCycler (Roche), which calculated Ct values. Delta Ct indicates the difference between the Ct of the integration and reference probe Ct values.

ddPCR of Donor Integration

To quantify integration efficiency by digital droplet PCR, 20 uL solutions were prepared containing 10 uL 2× ddPCR Supermix for Probes (Bio-Rad), 900 nM primers, 250 nM probes, 0.2 uL SacI restriction enzyme, and 1 uL genomic DNA. Identical primers and probes were used as the set used for qPCR. the 20 uL reaction was transferred to a Dg8 Cartridge (Bio-Rad) with 70 uL Droplet Generation oil for Probes (Bio-Rad), and loaded into a QX2000 droplet generator (Bio-Rad). 40 uL of the droplets were transferred to a 96 well plate and thermocycled according to manufacturer's specifications. Finally, the plate was loaded into the QX200 droplet reader (Bio-Rad) for droplet analysis and copy number quantification.

### Prime Edit Detection

To determine efficiency of prime editing alone, identical transfection conditions are carried out, but without the donor plasmid with a stuffer plasmid in its place (puc19). Three days post transfection, gDNA was extracted and purified as described above, and the edited locus is sequenced via next generation sequencing on an Illumina Miseq.

### Results

Validation of Prime Editing attA

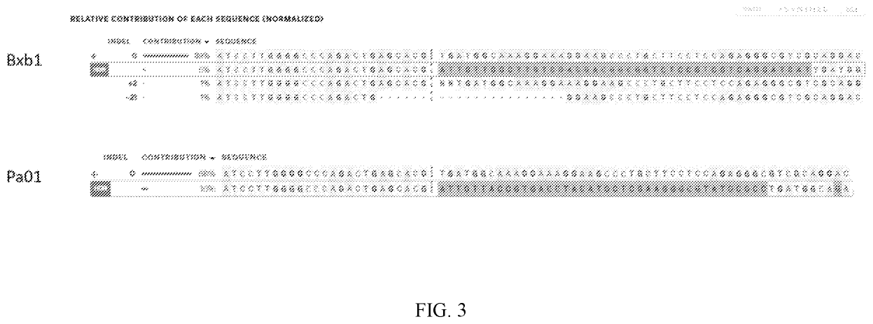

Three days after transfecting cells with plasmids encoding the prime editor, pegRNA, and ngRNA, gDNA was extracted and PCR was performed on target locus (HEK3). Sanger sequencing and ICE analysis confirmed that the attA for Bxb1 and Pa01, which is encoded on the pegRNA, can be integrated into the target locus (FIG. 3).

### PCR Validation of Donor Integration

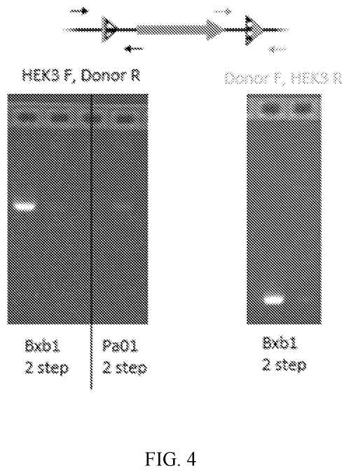

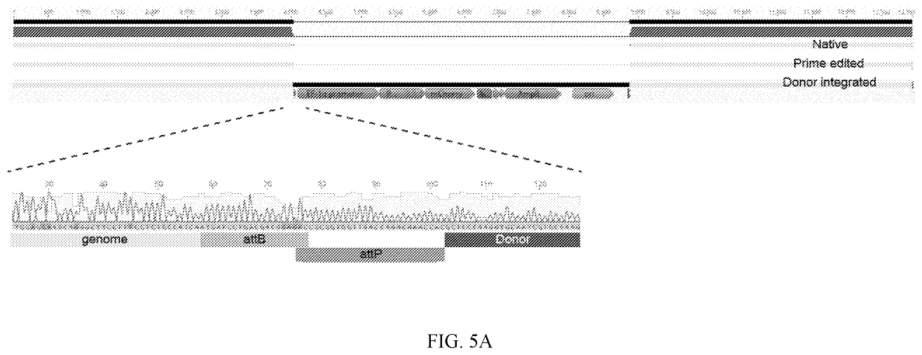

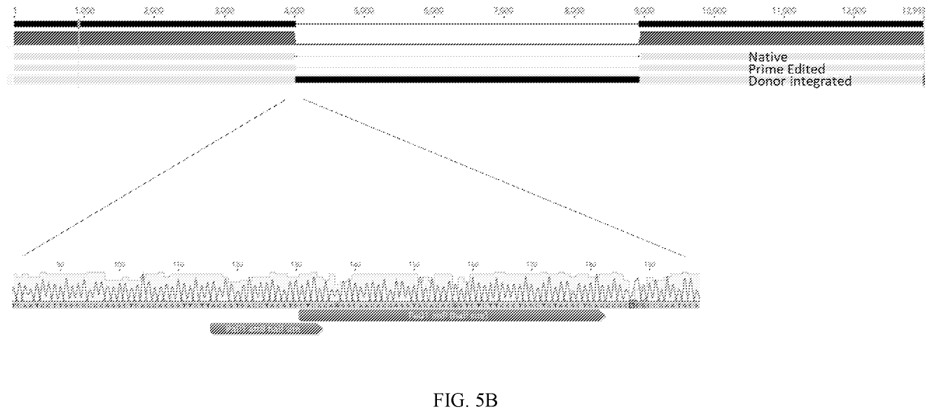

To directly detect installation of the attachment site at the target locus and integration of cargo into the attachment site, PCRs were performed across the integration junction. Via gel electrophoresis (FIG. 4) and Sanger sequencing of PCR products (FIGS. 5A and 5B), on-target donor integration mediated by the Bxb1 and Pa01 LSR-PE system was confirmed.

Evaluation of attA Length

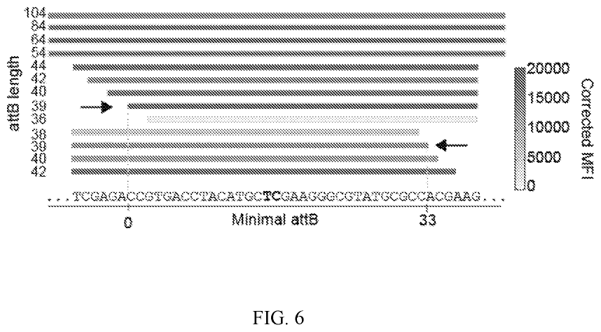

Truncation of attA site increased prime editing efficiency, but decreased LSR integration efficiency (FIG. 6).

qPCR of Donor Integration, 1 Step Delivery, 1 pegRNA

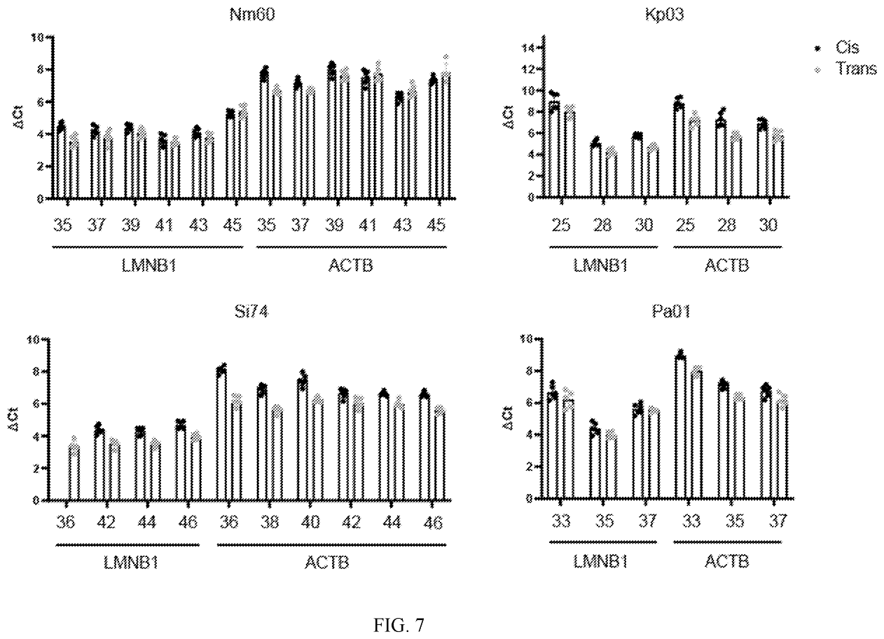

Via qPCR, we confirmed integration of the donor plasmid into the target loci for both LMNB1 and ACTB targeting pegRNAs, and utilizing Nm60, Kp03, Si74, and Pa01 as the recombinase in the LSR-PE system (FIG. 7). To get a rank order of integration efficiency, we calculated the delta Ct by subtracting the Ct of the probes targeting the integration junction from the Ct of a reference genomic region. Integration efficiency varies by loci, LSR, length of attachment site, and linker (cis vs trans).

ddPCR of Donor Integration at the ACTB and LMNB1 Loci

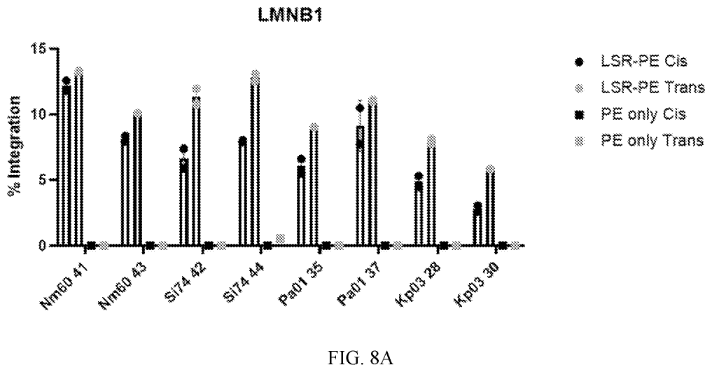

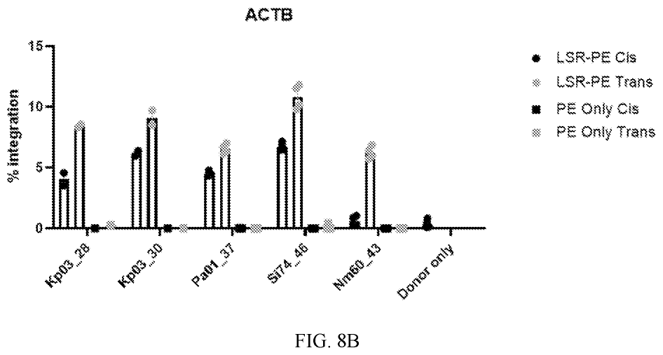

Absolute integration efficiency was determined utilizing a single pegRNA by performing ddPCR of the integration junction and normalizing to an unedited locus (FIG. 8A, 8B). All LSRs tested had detected LSR-mediated integration at the ACTB and LMNB1 locus, and no integration was seen in the PE-LSR-Donor and Donor only controls. Consistent with qPCR, trans delivery was slightly more efficient than cis delivery in all cases.

qPCR of Donor Integration, 1 Step Delivery, 2 pegRNAs

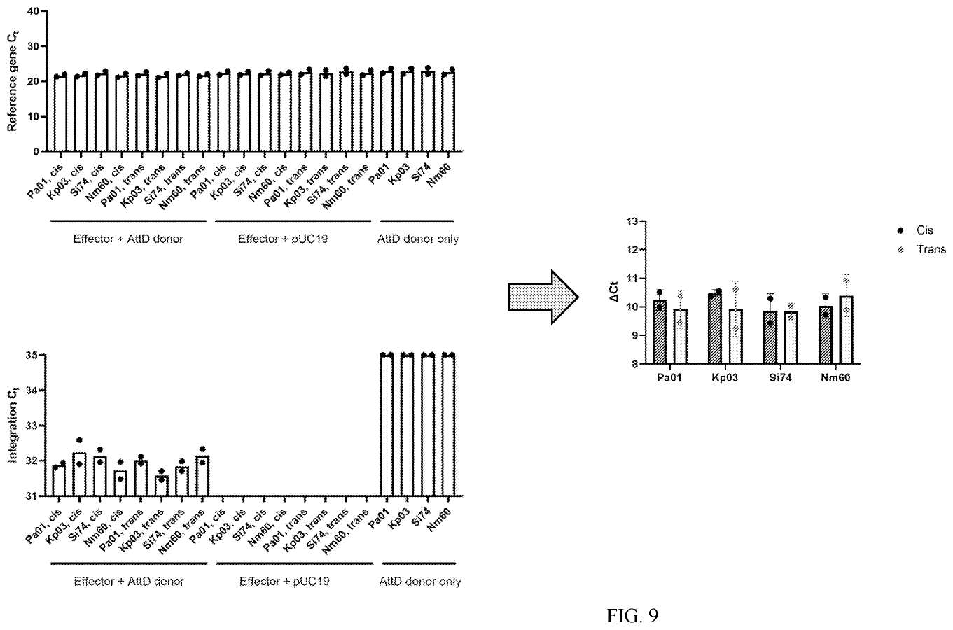

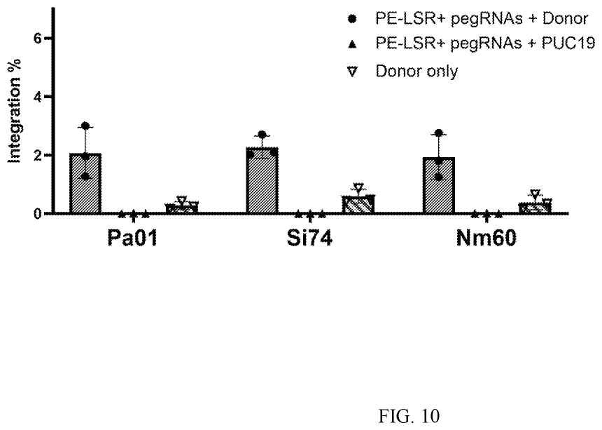

Integration into the AAVS1 locus was detected across all LSRs, in both cis and trans (FIG. 9 and FIG. 10). The no donor control had undetected integration, and the donor only negative control had a Ct>35, which is above the threshold for reliable detection and is considered undetected.

ddPCR of Donor Integration, 1 Step Delivery, 2 pegRNAs

Absolute integration efficiency of integration via 2 pegRNAs and LSR delivery in trans was determined by performing ddPCR of the integration junction and normalizing to an unedited locus. (FIG. 10) LSRs integrated at an efficiency of 1-4%.

### Example 2: Exemplary Sequences

spCas9 nuclease
SEQ ID NO: 1
DKKYSIGLDIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYNQLFEENPINASG
VDAKAILSARLSKSRRLENLIAQLPGEKKNGLFGNLIALSLGLTPNFKSNEDLAEDAKLQLSKDTYDD
DLDNLLAQIGDQYADLFLAAKNLSDAILLSDILRVNTEITKAPLSASMIKRYDEHHQDLTLLKALVRQ
QLPEKYKEIFFDQSKNGYAGYIDGGASQEEFYKFIKPILEKMDGTEELLVKLNREDLLRKQRTEDNGS
IPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRENASLGTYHDLLKIIKDKDELDNEENE
DILEDIVLTLTLFEDREMIEERLKTYAHLEDDKVMKQLKRRRYTGWGRLSRKLINGIRDKQSGKTILD
FLKSDGFANRNFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRID
LSQLGGD
SpCas9 H840A
SEQ ID NO: 2
DKKYSIGLDIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYNQLFEENPINASG
VDAKAILSARLSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDLTLLKALVRQ
QLPEKYKEIFFDQSKNGYAGYIDGGASQEEFYKFIKPILEKMDGTEELLVKLNREDLLRKQRTFDNGS
IPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDELDNEENE
DILEDIVLTLTLFEDREMIEERLKTYAHLFDDKVMKQLKRRRYTGWGRLSRKLINGIRDKQSGKTILD
FLKSDGFANRNFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQNEKLYLYYLQ
NGRDMYVDQELDINRLSDYDVDAIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRID
LSQLGGD
SpCas9 D10A
SEQ ID NO: 3
DKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYNQLFEENPINASG
VDAKAILSARLSKSRRLENLIAQLPGEKKNGLFGNLIALSLGLTPNEKSNFDLAEDAKLQLSKDTYDD
DLDNLLAQIGDQYADLFLAAKNLSDAILLSDILRVNTEITKAPLSASMIKRYDEHHQDLTLLKALVRQ
QLPEKYKEIFFDQSKNGYAGYIDGGASQEEFYKFIKPILEKMDGTEELLVKLNREDLLRKQRTEDNGS
IPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDELDNEENE
DILEDIVLTLTLFEDREMIEERLKTYAHLFDDKVMKQLKRRRYTGWGRLSRKLINGIRDKQSGKTILD
FLKSDGFANRNFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRID
LSQLGGD
SpCas9 N863A
SEQ ID NO: 4
DKKYSIGLDIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYNQLFEENPINASG
VDAKAILSARLSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDLTLLKALVRQ
QLPEKYKEIFFDQSKNGYAGYIDGGASQEEFYKFIKPILEKMDGTEELLVKLNREDLLRKQRTFDNGS
IPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRENASLGTYHDLLKIIKDKDELDNEENE
DILEDIVLTLTLFEDREMIEERLKTYAHLFDDKVMKQLKRRRYTGWGRLSRKLINGIRDKQSGKTILD
FLKSDGFANRNFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKARGKSDNVPSEEVVKKMKNYWRQ
LLNAKLITQRKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRID
LSQLGGD
SaCas9 D10A
SEQ ID NO: 5
KRNYILGLAIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDTGNELSTKEQ
ISRNSKALEEKYVAELQLERLKKDGEVRGSINRFKTSDYVKEAKQLLKVQKAYHQLDQSFIDTYIDLL
ETRRTYYEGPGEGSPFGWKDIKEWYEMLMGHCTYFPEELRSVKYAYNADLYNALNDLNNLVITRDENE
KLEYYEKFQIIENVFKQKKKPTLKQIAKEILVNEEDIKGYRVTSTGKPEFTNLKVYHDIKDITARKEI
IENAELLDQIAKILTIYQSSEDIQEELTNLNSELTQEEIEQISNLKGYTGTHNLSLKAINLILDELWH
TNDNQIAIFNRLKLVPKKVDLSQQKEIPTTLVDDFILSPVVKRSFIQSIKVINAIIKKYGLPNDIIIE
LAREKNSKDAQKMINEMQKRNRQTNERIEEIIRTTGKENAKYLIEKIKLHDMQEGKCLYSLEAIPLED
LLNNPFNYEVDHIIPRSVSFDNSFNNKVLVKQEENSKKGNRTPFQYLSSSDSKISYETFKKHILNLAK
GKGRISKTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMPEIETEQE
YKEIFITPHQIKHIKDFKDYKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYEVKSKKHPQIIKKG
SaCas9 N580A
SEQ ID NO: 6
KRNYILGLDIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDTGNELSTKEQ
ISRNSKALEEKYVAELQLERLKKDGEVRGSINRFKTSDYVKEAKQLLKVQKAYHQLDQSFIDTYIDLL
ETRRTYYEGPGEGSPFGWKDIKEWYEMLMGHCTYFPEELRSVKYAYNADLYNALNDLNNLVITRDENE
KLEYYEKFQIIENVFKQKKKPTLKQIAKEILVNEEDIKGYRVTSTGKPEFTNLKVYHDIKDITARKEI
IENAELLDQIAKILTIYQSSEDIQEELTNLNSELTQEEIEQISNLKGYTGTHNLSLKAINLILDELWH
TNDNQIAIFNRLKLVPKKVDLSQQKEIPTTLVDDFILSPVVKRSFIQSIKVINAIIKKYGLPNDIIIE
LAREKNSKDAQKMINEMQKRNRQTNERIEEIIRTTGKENAKYLIEKIKLHDMQEGKCLYSLEAIPLED
LLNNPFNYEVDHIIPRSVSFDNSFNNKVLVKQEEASKKGNRTPFQYLSSSDSKISYETFKKHILNLAK
GKGRISKTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMPEIETEQE
YKEIFITPHQIKHIKDFKDYKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYEVKSKKHPQIIKKG
M-MLV RT
SEQ ID NO: 7
TLNIEDEYRLHETSKEPDVSLGSTWLSDFPQAWAETGGMGLAVRQAPLIIPLKATSTPVSIKQYPMSQ
EARLGIKPHIQRLLDQGILVPCQSPWNTPLLPVKKPGTNDYRPVQDLREVNKRVEDIHPTVPNPYNLL
SGLPPSHQWYTVLDLKDAFFCLRLHPTSQPLFAFEWRDPEMGISGQLTWTRLPQGFKNSPTLFDEALH
RDLADFRIQHPDLILLQYVDDLLLAATSELDCQQGTRALLQTLGNLGYRASAKKAQICQKQVKYLGYL
LKEGQRWLTEARKETVMGQPTPKTPRQLREFLGTAGFCRLWIPGFAEMAAPLYPLTKTGTLFNWGPDQ
QKAYQEIKQALLTAPALGLPDLTKPFELFVDEKQGYAKGVLTQKLGPWRRPVAYLSKKLDPVAAGWPP
CLRMVAAIAVLTKDAGKLTMGQPLVILAPHAVEALVKQPPDRWLSNARMTHYQALLLDTDRVQFGPVV
ALNPATLLPLPEEGLQHNCLDILAEAHGTRPDLTDQPLPDADHTWYTDGSSLLQEGQRKAGAAVTTET
EVIWAKALPAGTSAQRAELIALTQALKMAEGKKLNVYTDSRYAFATAHIHGEIYRRRGLLTSEGKEIK
NKDEILALLKALFLPKRLSIIHCPGHQKGHSAEARGNRMADQAARKAAITETPDTSTLLIENSSP
M-MLV RT (D200N, T330P, L603W)5
SEQ ID NO: 8
TLNIEDEYRLHETSKEPDVSLGSTWLSDFPQAWAETGGMGLAVRQAPLIIPLKATSTPVSIKQYPMSQ
EARLGIKPHIQRLLDQGILVPCQSPWNTPLLPVKKPGTNDYRPVQDLREVNKRVEDIHPTVPNPYNLL
SGLPPSHQWYTVLDLKDAFFCLRLHPTSQPLFAFEWRDPEMGISGQLTWTRLPQGFKNSPTLFNEALH
RDLADFRIQHPDLILLQYVDDLLLAATSELDCQQGTRALLQTLGNLGYRASAKKAQICQKQVKYLGYL
LKEGQRWLTEARKETVMGQPTPKTPRQLREFLGTAGFCRLWIPGFAEMAAPLYPLTKPGTLENWGPDQ
QKAYQEIKQALLTAPALGLPDLTKPFELFVDEKQGYAKGVLTQKLGPWRRPVAYLSKKLDPVAAGWPP
CLRMVAAIAVLTKDAGKLTMGQPLVILAPHAVEALVKQPPDRWLSNARMTHYQALLLDTDRVQFGPVV
ALNPATLLPLPEEGLQHNCLDILAEAHGTRPDLTDQPLPDADHTWYTDGSSLLQEGQRKAGAAVTTET
EVIWAKALPAGTSAQRAELIALTQALKMAEGKKLNVYTDSRYAFATAHIHGEIYRRRGWLTSEGKEIK
NKDEILALLKALFLPKRLSIIHCPGHQKGHSAEARGNRMADQAARKAAITETPDTSTLLIENSSP
M-MLV RT
(D200N/L603W/T330P/T306K/W313F)
SEQ ID NO: 9
TLNIEDEYRLHETSKEPDVSLGSTWLSDFPQAWAETGGMGLAVRQAPLIIPLKATSTPVSIKQYPMSQ
EARLGIKPHIQRLLDQGILVPCQSPWNTPLLPVKKPGTNDYRPVQDLREVNKRVEDIHPTVPNPYNLL
SGLPPSHQWYTVLDLKDAFFCLRLHPTSQPLFAFEWRDPEMGISGQLTWTRLPQGFKNSPTLFNEALH
RDLADFRIQHPDLILLQYVDDLLLAATSELDCQQGTRALLQTLGNLGYRASAKKAQICQKQVKYLGYL
LKEGQRWLTEARKETVMGQPTPKTPRQLREFLGKAGFCRLFIPGFAEMAAPLYPLTKPGTLENWGPDQ
QKAYQEIKQALLTAPALGLPDLTKPFELFVDEKQGYAKGVLTQKLGPWRRPVAYLSKKLDPVAAGWPP
CLRMVAAIAVLTKDAGKLTMGQPLVILAPHAVEALVKQPPDRWLSNARMTHYQALLLDTDRVQFGPVV
ALNPATLLPLPEEGLQHNCLDILAEAHGTRPDLTDQPLPDADHTWYTDGSSLLQEGQRKAGAAVTTET
EVIWAKALPAGTSAQRAELIALTQALKMAEGKKLNVYTDSRYAFATAHIHGEIYRRRGWLTSEGKEIK
NKDEILALLKALFLPKRLSIIHCPGHQKGHSAEARGNRMADQAARKAAITETPDTSTLLIENSSP
M-MLV RT
(L139P/D200N/L603W/T330P/T306K/W313F)
SEQ ID NO: 10
TLNIEDEYRLHETSKEPDVSLGSTWLSDFPQAWAETGGMGLAVRQAPLIIPLKATSTPVSIKQYPMSQ
EARLGIKPHIQRLLDQGILVPCQSPWNTPLLPVKKPGTNDYRPVQDLREVNKRVEDIHPTVPNPYNLL
SGPPPSHQWYTVLDLKDAFFCLRLHPTSQPLFAFEWRDPEMGISGQLTWTRLPQGFKNSPTLFNEALH
RDLADFRIQHPDLILLQYVDDLLLAATSELDCQQGTRALLQTLGNLGYRASAKKAQICQKQVKYLGYL
LKEGQRWLTEARKETVMGQPTPKTPRQLREFLGKAGFCRLFIPGFAEMAAPLYPLTKPGTLENWGPDQ
QKAYQEIKQALLTAPALGLPDLTKPFELFVDEKQGYAKGVLTQKLGPWRRPVAYLSKKLDPVAAGWPP
CLRMVAAIAVLTKDAGKLTMGQPLVILAPHAVEALVKQPPDRWLSNARMTHYQALLLDTDRVQFGPVV
ALNPATLLPLPEEGLQHNCLDILAEAHGTRPDLTDQPLPDADHTWYTDGSSLLQEGQRKAGAAVTTET
EVIWAKALPAGTSAQRAELIALTQALKMAEGKKLNVYTDSRYAFATAHIHGEIYRRRGWLTSEGKEIK
NKDEILALLKALFLPKRLSIIHCPGHQKGHSAEARGNRMADQAARKAAITETPDTSTLLIENSSP
attA sequence
SEQ ID NO: 11
GTGGTGCTTGTTACATAACTCTTATGTATATTTTATATGGGGGTTATAGGGGGTACGTAGCACCGGTA
CCCCCCATAGTTGCAACGAACATTTGTGTACCAGGTGTAATA
attA sequence
SEQ ID NO: 12
TTCCGACGCAGTTTCCGACGAGTACGAGGACGAGGACAGACGTGCCTACCGGCAAGGTCAAGTGGTTC
AACAGCGAGAAGGGCTTCGGCTTTCTCTCCCGCGACGACGGCGG
attA sequence
SEQ ID NO: 13
GCGTCAGATCGACGATCGTCGGCAGCAGCGCGAGATAGAACAGCATGATCTTCGGGTTGCCGAGCGTG
ACCAGCGTGCCGGCGACGAACATCCGTATCGGCGATTGCCCGCGCGGCAG
SEQ ID NO: 14
attA sequence
CGGCTGACGCTGGCGCTGTCGCCGGCCACGCTGCCGAAGATGGGGTCGGTCTACGATCTCGCGCTGGC
GCTGGCCGTGCTGTCGGCGGAGAAGAAAACCGAGTGGCCCC
attA sequence
SEQ ID NO: 15
CCGGAAAGGTCCGGATGGAAGGCAAGGAATACGTCATGGTTGACGGTGACGTGGTGGAATTCCGGTTT
AACGTCTAGGCGGAAGCGCTACTAATGCCGCGCCGCCGCGAACTTC
attA sequence
SEQ ID NO: 16
GCTGGTCGAGGAGTTAATGAAGATCTCCAACATGTTCGGCTACGGTGAGGTAAATCAGTACAATCGTC
ACTCGACCCTAATGTCTGCCTATAAAAACATCAAAGATGAACATTTCCG
attA sequence
SEQ ID NO: 17
TCGTCGACGCCTCGCTGCCCGGCGGCGAGCGGCTCCACGTGGTGATCCCCGACGTCACGCGCACCGCG
TGGGCGGTCAACATCCGCAAGCACGTGGTCCGTGCGT
attA sequence
SEQ ID NO: 18
TCAAACGCTAGGTCTTCTGCTTCTAGCCATTTTTTGGCTTTTTTAATTGTGTCGCAATTTGGGATGCC
GAACATGGTAATCGTCATATTATTTCCTTTTCTTTTTATTTTT
attA sequence
SEQ ID NO: 19
AGAATGGCGCCCATTTTCTTTGAATCACAGAATAGCAAATTATGAGCGTACGTTTAGTGTTAGCCAAA
GGGCGCGAGAAATCTCTCCTGCGCCGCCATCCCTGGGTCTTTTCCG
attA sequence
SEQ ID NO: 20
CTTGACCGAATGGGACGAAGTAGATGTTTTTTGTGGCCATTAGGCGCATGAGGTTGACGCCATTAAGC
CCTAAAGCATCATTCGTCGAAACGGCCAATACGACAGGCTTGCC
attA sequence
SEQ ID NO: 21
GTGACCCGTTCCGCCGTCGAGAACGCCACCTCCGTGGCGCGCATGGTGCTCACCACCGAGAGCGCCGT
GGTCGACAAGCCGGCCGAGGAAGAGCCCGCGAACGGCCACCACGGCC
attA sequence
SEQ ID NO: 22
TAGCACCTATCTTATTGGCATTGATTGGTGCGTTAAATACTCCACCTGTAGTAGATACGTGTGTCCCA
ATAAATTTATTTATCTTTCTATTTTCCATTAAATAATTCTCCT
attA sequence
SEQ ID NO: 23
TGCCGCCACGCCCCACATTCAAGATGTCCCAATCCCCCAAAGTAGGGTTCGTATCCCTCGGGTGCCCG
AAAGCGCTGGTCGACTCCGAACAAATCATCACCCAGCTGCGCGCCGAGG
attA sequence
SEQ ID NO: 24
GGCTGGCCGGTACGTCCCGGCCCCCTGCGGAACATCTGCACGGCGCACGCCAAGTCGTAGGTGATCAC
GCCGTCGAGCGCCAGCGCTGCCACCGTCTTGGTGCCCATACC
SEQ ID NO: 25
attA sequence
GCTCGGCGGACGGCGACCCGTACCTGGCAAGCTGAACGCCGTGTTCCGAGTGATGTTCCACGAGCCCC
GCATCCCACCCAATACGGGAAACGCGATCCGCATGGTCGCCGGGACGG
attA sequence
SEQ ID NO: 26
GTTTTTTCATATAAAGTTACATCAGCACCTGCCTTTTTAGCTGTTATGGCTGCTGCACATCCAGACCA
GCCACCTCCAATTATTATAATCTTCATATTAGTCTTCTCCTTTCAAAAACA
attA sequence
SEQ ID NO: 27
ACGTAGTAGACATTTTTCTCGTCCAGGCGGTCCTTGGCGAGGCGCAGGCCTTCGCCGAAGCGCTGGAA
GTGTACGATCTCGCGCGCACGCAGGAACTTGATGACGTCGCTCA
SEQ ID NO: 28
attA sequence
CATGAATGCAGACCGAAAGTAACGTCGGCCAGGGGAAGCGGCGAGGTAAATCAAGGGTCATTGAAGTC
ATAATCATTTCAGTAGAAAAACCAGGTCTCGATTTTAAATGCAAA
attA sequence
SEQ ID NO: 29
CTACGGAATAGAGATAACACGAGGAGTGGTTAGAAATGGCTAAAGTTCTGGTGCTTTATTATTCCATG
TACGGACATATTGAAACGATGGCACGCGCAGTCGCTGAG
attA sequence
SEQ ID NO: 30
CAAATTTGACGGAAATGTTTTCAAACAACGGCTTACTGCCGAACTGCATGGTGACGTTACTGGAAACT
AACACAGGCGTATCCTGAAAAGAGATATGACAAACC
attA sequence
SEQ ID NO: 31
GCACCATGAATGCAGACCGAAAGTAACGTCGGCCAGGGGAAGCGGCGAGGTAAATCAAGGGTCATTGA
AGTCATAATCATTTCAGTAGAAAAACCAGGTCTCGATTTTAAATGCAAA
attA sequence
SEQ ID NO: 32
GCTTGGCGTTACCGGTCACTCCGCCCTGAATATGTGGTCCGGTATTGTCTTCAGCATTACATTTTTAT
TTTCGGCCATCGCCTCACCGTTTTGGGGTGGACTCG
attA sequence
SEQ ID NO: 33
CGACACCAACTGGCTTGGCTTCTGCTTGGATTTTACGCCATCCAGCCAATATGCAAGTGATCGCCGGT
ACGATGAACGTAGGGCGAATCAAGGAAATCGCTCAAG
attA sequence
SEQ ID NO: 34
GTATCCTTTTGGTAAAATTCATATCCTGCTGCGATGGAATAACATTACCAGAAGGATGATTATGCGCT
AAAACGATCCTTGCTGCACAATAACGTACAGCATAATGG
attA sequence
SEQ ID NO: 35
TCCGGGGGCCCCCACTATTCATATGAACGGCTCTCAACCTGTGCTAAAAAACGAAAGGACGGCATGCC
ATGAATATATTCGATCACTATCGCCAGCGCTACGAAGCTGCCA
attA sequence
SEQ ID NO: 36
TTTGCATTTAAAATCGGAGCATCATTTTTCAACAGAAACGACTATGAGCGCAATGACCCTTGATTTAC
CTCGCCGCTTTCCGTGGCCAACGCTGCTGTCCGTGGCTATCCACG
attA sequence
SEQ ID NO: 37
CGATCATCGCCGGACTGGTGGCCGCAGCGCTATGGACCGGGCGGCTGTCGAGGATCACGCGGTTGACG
TTACGCTCGTGCAGCCCGCGGTTGAGCTGCTGTTCCG
attA sequence
SEQ ID NO: 38
CGAGGATGAGTTATGAAGCTGGAAGAAATCGTAGCCCTTAGTGTAAAGCATAATGTCTCTGATCTACA
CCTGTGCAATTCCGCCGCACCACGCTGGCGGCGGCAGGGC
attA sequence
SEQ ID NO: 39
CCGGTTTCCCTTCGCACCCGCACCGCGGCTTCGAGACCGTGACCTACATGCTCGAAGGGCGTATGCGC
CACGAAGACCACCTCGGCAATCGCGGCCTGCTCAAG0
attA sequence
SEQ ID NO: 40
AACTGCCGGAGTTCGAGCGCAAGGTCCTGGAGGTCCTGCGCGAGCCGCTGGAAAGCGGCGAGATCGTC
ATTGCCCGGGCCAACGGCCGGGTACGTTTCCCGG5
attA sequence
SEQ ID NO: 41
TGATTGTTTTAAGTGGGACTTTTTATATTGCAAAAAATAAATGGCGGACGAGGTAACAGGATACCTCA
TCTGCCAATTAAAATTTGTTAATTTAATAATTAAATAAAAA
attA sequence
SEQ ID NO: 42
AACATGAGGTTATTGTTGCTAATATTAATAAGTTATATTGGAGGAACGTGTGCGTTAGAAGTCGTACC
ATTCATGTCCTTACGAGATAAATTAACTAAACACGTAT
attA sequence
SEQ ID NO: 43
GTGGCAAACCTTTTCAGTGCGTGATTGGCACCGGCCGGGTGATCAAGGGCTGGGATCAAGGGCTGATG
GGGATGCAAGTGGGCGGCAAGCGCAAACTGCTGGTGCCGG
attA sequence
SEQ ID NO: 44
ATAATGTACTGGTTAAAAGTAATTTATGAGCAATATATAAAAAATAATACTAAAAGTAATTAATTTTT
ATATATTGCTCATATTTAAAAAAAAATATAAATATAAGCT
attA sequence
SEQ ID NO: 45
GACAGTATCAAAATTTTATGGAAAATTTAACAAATTTAGTATCATTCATTTCAATCAAATATATTAAA
TTTGTTTATTAAATAGGAAGAAAAAGACGGTCAT
attA sequence
SEQ ID NO: 46
TAGGAAGGAATTTTTTTATAATACATAATTACATACAACATATAGTATGTAATGAATAACACTCAATA
TGATGTATGTAATAAATCCAAAGCTTAGTAATTAGAAT
attA sequence
SEQ ID NO: 47
CCAAGCAATACTATAGCTTCAGGTAAATAGGAACTTTAATACATTTATCTGAAGATATATGTATTAAA
GTAAAACTTTTTAATTATGATGTCAATTAATTCTTA
attA sequence
SEQ ID NO: 48
ACGTCAATTAAGTTTTCGTGTTTTTTATTGAATAGCCTTCTTAGTAGTTTCATTTGTAGTTCCTCCTT
CATTCGAAATCTTCAATTGACAAGGTTTCAATTCGTTTTTGGTAACGATATAAATAAAAGT
attA sequence
SEQ ID NO: 49
TAATTTAACAAGGCAGATAATTTAACCGCAGGGGACGCAAAGGACGCTAAATTTTTTTTATAATTTAC
TATTTTTTTCAAATAATTCTTATAAATATAATGGGGATGGGAAAATATTAAAAAATAATAGGAGA
attA sequence
SEQ ID NO: 50
AAGGAAGGGACGTGCTGGGAATCATGCCCACTGGGGCCGGAAAATCCCTTTGTTATCAAATCCCTGCC
CTTATGATGGATGGAATCACGCTGGTCATTTCCC
attA sequence
SEQ ID NO: 51
GGAGAATAGCGGGATCGAACCGCTGACCTCTTGCATGCCATGCAAGCGCTCTCCCAGCTGAGCTAATC
CCCCACATATTCGGTTGGTGTCCTGCGACGTGAGTTA
attA sequence
SEQ ID NO: 52
AATCACGATCTATTGGTCTCAATTCTCCATTCGTGATTGTATGGTTATTGTTGAATAAGTTAACAATC
GCGAATTTATGAAAATTGCCTATGCCCGTGTCTCAAG
attA sequence
SEQ ID NO: 53
CATTCATTGCAGATGTATGAGATGGAAAAAAGAAATAATTTTACTATCCTTTGTGGAAATGTAGGTTA
CTAAAATTACTTATATTTTCCACTTGATGACAAT
attA sequence
SEQ ID NO: 54
GTGGCAAACCTTTCCAGTGCGTGATTGGCACTGGCCGCGTGATCAAGGGCTGGGATCAGGGGCTGATG
GGCATGCAGGTGGGCGGTAAACGCAAACTGCTGGTGC
attA sequence
SEQ ID NO: 55
TGCGTCTAATCTACGGTTATAAGATTTTTTGTGTTTATGTTATGTTTACATGCTTAAACCTGACATAA
ATACTAATAAAATTCTATATGAGTGATTATTATT
attA sequence
SEQ ID NO: 56
CGGGCAGGGTCTACCTAAGCCTTTACATTTGTGTACATCTGAAATTGTTGCTTGTAGGTATCTCATAT
GTTTACAATTTGCACCCAAGATTCTTTCAGAGGGCGCC
attA sequence
SEQ ID NO: 57
AGCGGCACAGAAACCAAGCGACGAATTCATCAAGAAGATAACTTGAAAGAAATGGTGCCCGGAGGCGG
ATTTGAACCACCGACACGCGGATTTTCAATCCGCTGCTCTACCAACTGAGCTATCCGGGCACTTCAGG
TCCTTGAAGAAC
attA sequence
SEQ ID NO: 58
ATAAATTTCTGTAGTTATTTTTCAAAAACCGCATCATTAACTGATAAGCAGAAGCATATCACAAATAA
AACTAAAAAAACGATGTTGAACAATAATATTCATTATGAATTTTTTGAGTAAATCTTAGG
attA sequence
SEQ ID NO: 59
ACGCTGTGCTCTTTTGTTTTGTAATTTTTCGTATTTACGTGAACTTTATATGTGTAAATGTAACATAA
ACACTAATAAAATTCTATATCTAATACTTCTGTAA
attA sequence
SEQ ID NO: 60
AATAATTTTAATTTTTTATAAAAAATATTCATATATTCTTTATATTAAAGTTTAGATATCTAAAAATA
CTTTTAGAATTTATTATATTATGTTAATTTTTTTATA
attA sequence
SEQ ID NO: 61
TGTCTGAAATAACAGACACTAAATATATAAGTGTTTTATGTACATTTATTGAAATAAGTGTAAGTTAA
ACACTCTATTTTTTAAATAAAATTTCCATGTCCT
attA sequence
SEQ ID NO: 62
GCCGGCACCAGCAGCTTGCGCTTGCCGCCGACCTGCATGCCCATCAGGCCCTGGTCCCAGCCCTTGAT
TACCCGACCGGTGCCGATCACGCACTGAAACGCCTTGC
attA sequence
SEQ ID NO: 63
TGAAAAGCTATTTTATACAACGGGGGCATAGCTCAGTTGGTTAGAGCATCTGACTCTTAATCAGAGGG
TCTAGGGTTCGAATCCCTATGCCCCCATTGGGTGCCAAACCC
attA sequence
SEQ ID NO: 64
GCACGAACAAGCGACGCACCCCGCCCACCTGCATGCCCATCAGGCCCTGGTCCCAGCCCTTGATCACG
CGCCCTGTGCCGATCACGCACTGGAAAGGCTTGC
attA sequence
SEQ ID NO: 65
GTGGCAAGCCATTTCAGTGTGTGATCGGCACCGGTCGCGTCATCAAGGGCTGGGACCAGGGCCTGATG
GGCATGAAAGTCGGCGGCAAGCGTCAATTGTTCGTCC
attA sequence
SEQ ID NO: 66
TCAGCAGCGCGGCCACCTGCTCGTCGGCGAAGGACTCGTGCGCCATGACGTGACACCAGTGGCAGGCG
GCGTAACCGACCGAAATCATGATCGGGACGTCTCGGCGGCGG
attA sequence
SEQ ID NO: 67
AAGTTAAAGCGGAGGTTTCTCTGTACGACCCCATTGGTGTAGACAAGGAAGGTAATGAAATAAGTTTG
ATAGATATTTTGGGTACCGACCCGGAAGTGGTGGCGGACATGGTG
attA sequence
SEQ ID NO: 68
ACTCCAAGCAGGTAGGCCGTTTTTCTAAACTATGCTAAGCAATTTCTTTAATTAAAGTTTTTGCTTTG
TATGGTTTAGGTGATAAGCTCTGCACCCCTTTAA
attA sequence
SEQ ID NO: 69
TGGAATCGGTGGGAATGAAAATCACCGTTAATTATGGAGAAAAGGATGGGAAAATTGTCAAGGGTTCT
AAGACCTACGGCGATGTCAAGAAAGAAGTGACAGA
attA sequence
SEQ ID NO: 70
CGAAAAAAAGGAATACCTCTATAAAATAATATGGGTATTCTCTAATATTTATTTCTATAAATATAGAG
AAATACCCATATTTTCGCATATAAAAGAATAAATTAT
attA sequence
SEQ ID NO: 71
TGTCTTGTAAGCACCCATCCCTGTAGTTATGCACTGTACATTTGCTTTCAGTCTAATGTATAGTGATA
ACTTACATTTTATGGTATGGTGTAACAATGAGAAA
attA sequence
SEQ ID NO: 72
TGTCGCTGCATCGAAGCGGGCCCCTGTAGCTCAATTGGATAGAGCATCGGACTTCTAATCCGACGGTT
GCAGGTTCGAGTCCTGCCGGGGGCACTTCCAGGACGCTGTTTGGCACACCAGCGTCCTTCTCGGTAGC
GACCGCCATGA
attA sequence
SEQ ID NO: 73
ATATGAATCTATCCTTAGTAGCTATTGATGAGGCTCATTGTATTTCGCAGTGGGGACATGATTTTCGG
CCAAGCTATTTAAAAATTAAAACCCTTTTAAAAGAGTT0
attA sequence
SEQ ID NO: 74
TATTACGCTGATTTACAGCTGATTATTTAATCAATAATGTTTTTAGTTTAATCAATTAAACTAATAAC
TTTTATGATTAAATTTAAAGTGCTATATTTGTGCTGAA
attA sequence
SEQ ID NO: 75
CCAAAATGATTTTTGTTTCATTCTAATTTTCCAATTAATCATATTCTTATCTCCTTTTATCCAAAATA
AAAAACGACTAAAAAATTAGTCGTTTAAAATTATTCAATGGTCAATGTTGGAGATCCTGAATA
attA sequence
SEQ ID NO: 76
CGTAGGCCGCGAGCTCCTCCTCGACGACAGGGCGCGGGCCGATCACGCTCATCTGGCCGGCCAGCACG
TTGAGGAACTGAGGGAGCTCGTCGAGGGAGGTCTT
attA sequence
SEQ ID NO: 77
ATAATTTATATATTAATAAAATTATACTCTTAAAACAAAAAAGAAGCTCTAAAAAGAGCTTCTTTTTA
TAATTAAATTTATATTATTTTAATCTTTCTTCTAAC
attA sequence
SEQ ID NO: 78
TTGCCCGGTATCAGGAAACTTTACTTATGATAGTTGACACTGCTTATTCAACACATGTGGAGTGTGTG
GTGTTGTTGGTTAGGAATGACAGATAATTGAGGTATACTGACGTGT
attA sequence
SEQ ID NO: 79
ATAAATTTACTGAGGCTTTATCGGTTAAGGTTCCTTGAAAAAACAACAAAATCAGGTTAAATTAGGTT
TTTTAAGGCAGATGCAATCTGTTTGTGCTGCCAATA
attA sequence
SEQ ID NO: 80
AAGTAGGCGGTCATGGGTTGTATTTATCAATATTTTAAAATTAGTGAAACTCCACTTTCCCTTAGTTA
AAGATATGAACCCTATTTATTAGTCTACTTTCCCAATA
attA sequence
SEQ ID NO: 81
CCTGTGATATATGAATTAATATCTTATTCCCTTCAGCATATCCAAACATCTCATTTATATATTTAAAT
TTATCAATATCTATATATAATAAAGCATACTTTACTTGAGTATTTTTACATAGT
attA sequence
SEQ ID NO: 82
CTCCTGTTGCTCCTGTTGGGCCTGTTACTCCATCTGCTCCTGTTGCCCCTGTTGGGCCTGTTGGGCCT
GTTACTCCTGTTGCTCCATCTGCTCCTGTTGCTC
attA sequence
SEQ ID NO: 83
CATATGAGTCAAGCAAATAAAGTGTATCTCATTTTTAAACGAAACATGTATTACATTTAAAAATAGGA
ATTTTACCCCATATGATATTATACATTAACTAAG
attA sequence
SEQ ID NO: 84
GAATTTTATATATTTTAAATATGATAGTTTAAAATTATGAAAGAATGATAAATTCCAGTTTGTAGGGT
AATTTTGATAAATAATATTAATAAGTATGAGTAAAAAGTAGGAG
Sh25 integrase
SEQ ID NO: 85
MKVAIYTRVSSYEQATEGYSIHEQERKLKAFCEVQNWNEFKVFTDAGVSGGSMNRPALKRIMDNLEYY
DLVLVYKLDRLTRNVKDLLEMLETFEKYNVAFKSATEVFDTTTAIGKLFITMVGAMAEWERATIRERA
LFGSRAAVREGNYIREAPFCYDNVDGKLVPNKYKWIIDYLVEQFKHGVSGNEIARQMNVKKVNVPKVK
KWNRTSIIRLMKNPVLRGHTKYGDMYIENTHEPVLSESDYKRIIDVIENKTHRSKVKHHAIFRGVLTC
PQCHNKLHLYAGKITDKKGYSYEVRRYKCDTCSKDKNVQTISFNESEVEDKFIELLKTYDMNKFKVDI
VEESTPKLDYDIDKIMKQREKLTRSWSLGYIEDDEYFSLMDETKEILDEVERAGTEVESTQTVTNEQL
NMIDNILIKGWSKLNVEQKEELILSTVKEIVFDFVPRKYNENGKVNTLNIREITFKF
Si74 integrase
SEQ ID NO: 86
MQPNLRYLACLRLSADSDGSTSIEWQRGVIRHHVSSPHLSGVLVGEAEDTDVSGSLSPFKRPKLGKWL
TAKADEFDVIIAAKMDRLTRRSMHFNELLEWAQQNGKFIVCVEEGFDLSTPQGKMMARMTAVFAEAEW
DTIQARILNGVQTRLENRSWLVGAPPTGYRIKTVEGGKRKILEIDQDFYPYVEEIFRRIREGQSTHRI
ARDFNGRSVLTWGDHLRKLKGEEPKGTQWQATIINKFIRSSWVPGLYTYKGEAVLDDQGDPVILPETP
LATMDEWTDLVDRIKPAPKPEGATGGSRNSAKSLLSGVAHCGECGAPFTSLMDSGYKRKDGTKVPGHR
RYRCSNKFKGGDCKNGSYVRADVLDSWVDQAIRDSIGQEDMYERAGKGPSQARELQETKARLAKLEAD
YESGKYDGEGQDESYWRMNKNLSAKVAHLAKQEAERANPTFKATGKKYGEVWEAKDQEDRRDFLRTYG
VKVFVWGEGADKKDRGYAMNLGDIKTMAEELFPNRDRARFKLVHTHNAPEGYLSKIGIAVGLLKYGHP
LEVKLRSPENS
Bm99 integrase
SEQ ID NO: 87
MAKKPKAKVYSYLRFSDPKQAAGSSADRQMEYAARWATEHDMQLDATLTLRDEGLSAFHQRHIKQGAL
GVFLRAIEDGRIQPGSVLIVEGLDRLSRAEPIQAQAQLAQIINAGITVVTASDGREYNRERLKAQPMD
LVYSLLVMIRAHEESDTKSKRVKAAIRRQCEGWVAGTWRGIIRNGKDPHWVRLGEHGKFEHVPERVLA
VRTMIDLFLEGHGAIEITRRLTEQNLYVSNAGNYSVHMYRIVRNQALIGEKRISVDGEEFRLDGYYPP
ILTREEFAELQQTMSERGRRKGKGEIPNIITGLSITVCGYCGRAMTTQNSKARAPKGKSVVRRLSCPM
NSFNEGCPIGGSCESEIVERALMRYCSDQFNLSRLLEGDDGTARRTAQLAVARQRASDIEAQIQRVTD
ALLSDDGKAPAAFTRRARELETQLEEQRREIEALEHQIAASSAHGIPAAAEAWAQLVDGVLALDYDAR
MKARQLVADTFRKIVVYQRGFAPIDDAAADRWKRSGTIGLMLVTKRGGMRLLNVDRRTGCWQAEDDLD
PSLIPSDGLPMLPLDA
Me99 integrase
SEQ ID NO: 88
MKAVIYTRISKDREGAGLGVERQRADCAALADRLGWQVVGTFSDNDLSAYSGRHRPGYAAMCEALEAG
EAQAIIAWHTDRLHRRPVELESFIGLCERRSIQVRTVRAGTLDLSTPSGQMVARMLGAAARHEIDHAI
ERQKRAKKQAALDGRYRGSRRAFGYERDGLTLCDAEADAIRTAAERVLSGTSLSQVARDWNAAGLRTA
FGGKAFTSREVRRILLRPRNAGYSLHEGKRIPNAQWPPIITTDTFAALEALLRDPVRSKHLAFERKWM
GSGVYLCGKCGAKISTASQKGTGKSWRPTYVCSASKHLGRVADTVDEYVTEAVLERLSRPDAPILLGG
NKVDVADLTSRRDGYRARLDELAAMFAAGDIDAGQLKSGTTELRRKLDRVDAELAAARASSVLADLVL
SGDDLRDTWAAIPPGGKGKVIDALMTVTIEPTRRGRRPGGSYFDPESVTIRFKGVGEHRLDDGQLIGA
Ma37 integrase
SEQ ID NO: 89
MASPPRNAALYLRISLDQTGEGLAIDRQREECERIAAQRGWTVVGVYEDRSISATQANKKRPGYEQLV
SAYQAGQFDAMVCYDLDRLTRQPRQLEDWIEAAEGRGLALVTANGEADLTTDGGRMFARVKLAVARSE
VERKSARQRTAAHQRASLGRPPLGTRLTGYTPKGETIPAEAEVIRRIFKLFQAGETLRSITRMLTEEG
VTTRRGNAWNPSTIYGTLTNPRYAGRAVYQGKPTGQLGNWEPLVSPEVEDLIQARLADPRRKTNRVGT
DRKHLGSGLYVCAVCEQPTTSWSQGRYRCKDSHVNRAQSQVDSYVLDTIAARLRRGDIATLLAPAKAD
LAPLLDDIERLTARQATIDADYDAGLIDGTRHAAATATVRAELIAVQQQMAAADKGSALGELLTSPDP
AQAFLDAGLMTQRSAIDALAVVRLHRGHRYSRTFDPETVEVDWRRPR
Nm60 integrase
SEQ ID NO: 90
MSRPTGLTIDIYLRKSRKDLEEEKKASESGETYDTLERHRRTLFAVAKKERHNIANIYEEVVSGESVS
ERPQIQAMLRNLETSHIEGVLVMDLDRLGRGDMLDQGLLDRAFRYSGAKILTPTEVYDPESETWELVE
GIKSLVAREELKAITRRMQRGRVASAGEGKSISKVPPYGYLRDENLRLYPDPETAWVVKKMFEMMRDG
HGRIAVAQELDKLGIKPPNDKRRSWAPSSITAIIKNEVYLGTIIWGQVKYSKRNGKYKKTKLPRSKWT
IKENAHEPIVSRELFEAANRAHTGRWRPSTNATKTLSNPLAGVLKCDVCGFTMLYQPRPNRPNDFIRC
TQPTCQAVQKGATLALVEQHILDGLKQFAQELELQTEVPELDNDKDIAVKKSLVGNKQEEIAQLETQK
SKLHDLLERGIYDVDTFLERQQNLNNRINGLQDDIRNIESEIKKEEVRNSSVLNLLPQLQTVISEYEN
ADTESKNRLLKSVLEKATYLRKKEWTKRDQFIIQLYPKI
Cc91 integrase
SEQ ID NO: 91
MSRRRALAPVPDTPPRAVAYLRQSTYREESISLELQETACRDYAARHGYQIVAVEKDPGISGRTWNRP
AVQRVMEMIESRDADVIVLWRWSRLSRNRKDWAIAADRVDVAGGRIESATEPNDTATAAGRFARGVMT
ELAAFESERISEQWKEVHTSRVARGLPPGGKLPWGWRWVDGAVRPDPETAPYIVEAYRRYLAGAGNRD
LADWFNGSGVRPMHAKEWYFSTITQCLDSPIHAGLIAYHGQTLPGAHDGIIDVATWEAYRRERERRAG
ERQVKRRYLLSGIAHCPCGEPMLGFTQDKEGRPRTGRTRSPWSCYRCSSLGKPEGHGPWNISLRFVEP
VVMDWLHQVAADVENKVPRAAARRDDAHRESQRIAREITALDAQLTALTGHLASGLVPEAAYVTTRDE
ILARRAELERGLAEAERLVLHVPDDPSAIAREALADWDTLPIETKRATLRQLIRTVLVDYEHRTAHVV
PVWEPMHNEG
Vh19 integrase
SEQ ID NO: 92
MKTAYSYIRFSSSRQADGDSIRRQTELARAYAEEHELDLQDVSISDFGVSAFRGSNATEGALGAFLKL
VDEQKIDSDCYLLVESFDRLSRQAVEDALALLLQVVNRGITLVTLSDNHVYKRGELDMTMLILSIVTM
SRANEESEIKSQRTSAAWSKARIDALNGIKVKNSRLPDWLSWNEDKTDFVVNQPAKATIQRMFELSAG
GSGIEIIAKTLNSEGLKTFKQFKEWRSSGVSKLLRNRAIVGEFQFYRKDAKGNRVAVGEPIAGYYPEV
VSKQLFLTVQQGMDLRNRRGSGNRAGQFTNLFTGLIRCGECGSAVVTSSQTTKTPQGYLKCTMRCESK
ARMNYKYTEPQVLSALGSLQSVIEKYRTPISDETASIELDVRALDEKVTTFESLLDNAFTPKLAERLQ
EAELQLANAKALLESERQRVSAEQSREQIVLGLEPLESTDDRRAFNSKLRTVIDRIEMIEHPHEKGSA
LVYFINDCPVMEQHFTKLKGAHGTSSEVYELHSDDVPSHLGRTTSKKPLEFIIEESGDVLSEMPAQ
Cs56 integrase
SEQ ID NO: 93
MEKRKLYSYIRWSSDKQAKGSSLQRQLETARRVAHENDLELVEIIDAGLSAFRSKHLEKGSLGAFIEA
VKVGQIASDSWLTVESLDRISRDAILKAQGLFMELLELGITIYTGMDNRIYTKSSVTDNPMELMLSIM
TFSRANQESMVKSQRQKSATQLKINKFNASVRADNGYPHAIRNSGANVFWSDVSDGTVRPHEYYFPIA
KLIVSLRLKGWGYMRIAKHLNENYTPPKGTAKRKHFKDLWSPKLICNFLQSRTLMGEKSIRVDGVEHI
LKGYYPQVISENEFYSLQNVINRKLANEPNNYIPLLTGIRRFKCGCGAAMVAFFQYDKIRYRCDGMKS
LDKRMHCGAKSENGASIENAIVQICADKIFKDVKTHTSNVGAIQAMLVEAKRKYNRGMDMLLEDTAPQ
GLNIKLIELEKQIEELQKKLNDELVTEAGVNSSVSWGAVPKDYRDVENTERLEIKQKIQASTLSIVCT
TIKTKGHLFEIKENDGDVIRFFSDKRRVHVDVHSINNAEIMQSEGIILHDHLDYLVGPEEFIERIRQK
HLQMKALQERTLDDIFDI
Bt24 integrase
SEQ ID NO: 94
MKTAIYLRKSRADLEAEARGEGETLAKHRSTLLKIAKEMNLNVLSVREEIVSGESLVKRPEMLALLEE
IEDNKYDVVLCMDMDRLGRGGMKEQGIILETFKRSNTKIMTPRKTYDLNDEWDEEYSEFEAFMARKEL
KIITRRMQRGRVASVEAGNYLGTHAPFGYDIHRLNKRERTLTINPEEASIVRMIFDWYANEDMGANAI
RSKLNDLGYKSKLGNEWNPYSILDILKNNVYIGKVTWQKRKEVKRPDAVKRSCARQDKSDWIIADGKH
EPIIPESLFEQVQEKLNSRYHIPYNINGIKNPLAGIIKCAKCGYSMVQRYPKNRKETMDCKHRGCENK
SSYTELIEKRLLEALKEWYINYKADFEKHKQDDKLKETQVIQMNEAALRKLEKELVDVQKQKNNLHDL
LERGVYTVDMFLERSNVVSDRITEITSTMENLKKEIKTEIKKEKVKKDTIPQVEHVLDLYFKTDDPKK
KNSLLKSVLEKAVYKKEKWQRLDDFELVLYPKLPQDGDI
No67 integrase
SEQ ID NO: 95
MPESPPRALIVIRLSKVTDATTSPERQLAECRAICEKRGYEVVGVAEDLDVSGAVDPFDRKKRPHLAR
WLHGEHLDDNGEPVPFEVIVVYRVDRLTRSVRHLQKLVAWADDHKKLVVSATEAHFDTSLPFSAVLIA
LIGTVAEMELAGISERNASAARHNIQAGRYRGSTPPWGYVPSNDTGEWRLVRDKEQADVINEVARRVI
EGEPLQKIAHDLTRRGILTPKDNFAKQRGREIKGREWSVTQLKRSLLSEAMLGHAVSGGAAVRNDDGS
PVVRSEPILSREIYDRVAAELSSRAKRGEPNKRTSSLLLGVISCGNPCLHKQQGHECPEGCSGTCDEP
VYKFNGGSHSQFPRYRCRTMTRAYKCGNRTIRADQADAQVERTILALLGSSERLERVWDAGEDHSAEL
ADINDELVDLTSQLGVGAFRAGTPQRAKLDARIASLAERQAQLSSEAVVPAGWKLLPTGELFGDWWSR
QDLTARNVWLRSMGVRARFKRDDKTLYIDLGNLNDLISGLKPGGTAQRVRGGLQAMERNGIQGMVFSD
ADSEVMPAPAAGYMWIQPVEGVWVYTSEALLAAAAERQALRREKIEDEFAYGPGDFDEDWD
Fm04 integrase
SEQ ID NO: 96
MRDSKSKTVIIYNRVSTIMQDKNESLTEQTDECIRYCKINGYEIYKILKDVKSGTKDDRAGYLELKKH
IRRRDFDILVVLETSRIARKMKELVLFFSLLNENNIEYISIREPNYNTTTPDGKFAMNIRLGLIQFER
DNTAERVTDRLYFKASKGQWVNGKPPIGYKLVNKRLEIDEEKAEIVKNIYEDFLNGYSLNQINNKLQF
SWGSKQVKRILINPTYKGYIRYGTRSNRKKNNREAFIVKGWHEAIIPEDKWEKVQEMYKSLNRKASNT
KPTLLSGLLKCKECGNNYIRKRGGSYDKNLYYGCNLNNLRYSDKFFYKDIPECSSATIKGDLLEKAVI
DTLKKQINDLNENDIEIDKKRQVNIKQIDSSINKFKNRLNKIYELYIEDEIPKDKYLKDKKDIEARII
SLEKQKKSFGEIEVEKSNNEMIQEYFSKIDLSNIEEANRILKIIVNKIVVYKKKKTPKDDFEVEIYLN
I
Bu30 integrase
SEQ ID NO: 97
MAAKSRVYSYLRFSDPKQAAGGSIDRQLEYAARWAADREMELDASLSLRDEGLSAYHQRHVRQGALGV
FLRAIEDGQVPAGSVLVVEGLDRLSRAEPLQAQAQLAQIVNAGITVVTASDGREYNRERLKAQPMDLV
YSLLVMIRAHEESDTKSKRVKAAIRRQCEGWIAGTWRAPIRVGKDPHWVRETEGGGFELAADRASAVR
LVIEMFRQGHGAVRIVRELAERGLQISETGRTHSSNIYKILANRMLIGEKSVEVDGETYQLDNYYPAL
LTPAEFADLRYLAEQRGRRKGKGEIPGVVTGLGITYCGYCSAAIVAQNLMGRKRLPDGRPYPGHRRLH
CVTYSQSVGCKISGSCSVVPVERALMLYCSDQMNLTRLLEGDAGTASISAQLASARQRVAELEAQIRR
VTDALLFDDGDAPAAVLRRVRELESQLASERRDVESFEHHLAASASNVAPAAADAWRELVQGVEQLDY
DARLMARQLVADTFSRIVIYQSGFQPETDHGTIGVVLVGKRGNTRMLNVDRRTGEWRSAEDIELSGLA
TIPLPA
MA5 integrase
SEQ ID NO: 98
MRVLGRVRLSRLTEESTSVERQRELIKKWSEMNDHTIVGWAEDVDVSGSIDPFEAPELGPWFQEDKRG
DWDILVAWKLDRIGRRAIPLNKVFGWMLEHEKTLVCVSDNIDLSTWVGRLVANVIAGVAEGELEAIRE
RTKASRKKLLESGRWTGGPVPYWLIPEKLPEGGWVLSLNTETAPILRRAIDEVLDGTAVHTVAERLND
QGVPSPGGKKWTSQTLWRILQHKYLKGHSTDRGKTVRDSSGVPISNCEALLTPSEWDRLQAVLTQWKL
PETSNRVKNTSPLLGVVVCYICDKPLYYRSYTRNYGKGLYRSYYCRNHRTPGIKADMLDEYLEENLMR
EVGDKNVLERYFVPAENHQIELDEAIRATEELTALLGTMTSATMRSSLTAQLAALDSRIASLEKLPTS
ESRWEYRELPRTYREMWESDDDPQFRRELLLKSGITLAATMTGGQKLHLHIPDDILERMALKGE
Rh64 integrase
SEQ ID NO: 99
MESASPGLRVLGRLRLSRLTDESTSIARQRETIQRWAEIQGHTVIGWAEDADVSGAVDPFDTPQLGQW
LNHRVEDYDVIAAWKLDRLGRNAIQLNKLFGWCIDHNKTLVSCSESIDLGSWSGRMLAGVIAGLAEGE
LEAIRERARSSRVKLREAARFAGGKPPFGYRKVRRDGGGWMLEIDEPAAELVGKIVADVLDGKPVSRV
VMALNEGGYRTPRDYYETVKAGKPALKLAAGERRNSEWRSTALRNLLTNPALRGYVHHKGQIARGDDG
MPLQLAEEPLVDADEWEGLQAIFNGHRERRSHYRRPDASPLVGLVYCYWCHSPLWHNRNVSRGHEYFY
YRCPNIQKHERASMIPADQAEKAVADSFLDQAGDLPVMERVWVPGDSTERELRDAVAALDELTEVLGT
LGSATARQRITRQITALDERIRDLEAQPVREARWEYKQLGGTYREAWESLGEAERWQLIMKSGMTFAF
GLTDRGRGPNSVWVSSVYTPEPLKQTLVSGVTQRRTLADADPHRDVNSADHTKSLPEHWATMRKHGVE
GIEVHEVKSVFVSKPGERVEIPHWLHEVGISEITNDDDVRIIWTQDGRGWYQHSDGEWIEVPLGELEE
Cb16 integrase5
SEQ ID NO: 100
MLRIAIYSRKSVETDTGESIQNQIKLCKEYFKRQDPNCIFEIFEDEGYSGGNINRPSFQRMMELVKIK
QFDIVAVYKIDRIARNIVDFVNTYDELDNIGVKLVSITEGFDPSTPAGKMMMLLLASFAEMERMNIAQ
RVKDNMRELAKMGRWSGGTPPKGYTTKKVIENGKKITYLDLIDDEAYIIKDAFKLYAEGYSTYKINKH
FKEKGIRLPQKTIQNMLNNPTYLISSKESVDFLKNKGYTVYGEPNGFGFLPYNRRPRTKGKKSWNDKS
QFVGVSKHEGIIDLPLWIEVQNKLKERTVDPHPRESNFTFLSGGLLKCSCGSSMFVHPGHTRKDGSRL
YYFRCMKNNGNCSNSKFLRVDYAESSILEFLESISSKEKLTEYQKKKKPRLDESIEIKNLNKKIRDNS
KAIDNLIDKLMILSNEAGKVVATKIEELTKQNNILKESLLEIERKKLLSGLEDNNLNILYNEIQNFIQ
TEDISLRRLKIKNIIKYITYNPQNDSLQVELVD
uCb4 integrase
SEQ ID NO: 101
MNAIYARQSVDRADSISIESQIEFCQYEMRGEQYKVYTDRGYSGKNTDRPAFAEMMNDIENGVIGKVV
VYKLDRISRSILDFSNMMEQFGKHKVEFVSTTEKFDTSSPMGRAMLNICIVFAQLERETIQKRVADAY
YSRSQKSFYMGGRVPYGFRLIPTTIEGIKTSMYEINAEEAEQVQLIYELYSKPECSYGDIIRYFQAHG
ILKNGKPWGRTRLADILRNPIYVRADPSVYAFYRDQGAIMANDPADFIGTNGCYYYKGQDSAGRKQMN
LEGNHLVLAPHEGIISSDLWLKCRVKCLEAQQIKPYQKAKNTWLAGKIKCGACGYALVDKHYSTTRSR
YLLCSNKMNAKACEGPGTIYTDEFEQIIYNEMQKKMAQFKKLRRCKGKRVNPELTALNIQLTQVETEI
SSLMDRLSAADDTLFRYISGRIKELDGKKQELMKRISERKLHKEADYTEINNHLTMWDELSFDDKRQT
VDQLIRVIYATSDSIKIEWRI
Ec03 integrase
SEQ ID NO: 102
MGRSVITYLRYSSAIQGAEGADSTRRQNDLFKQWLKKNSDAQVVASFSDEGLSGYKGKHLTGQFGDML
ARIESGEFPEGTILLVESIDRIGRLEHLETEALMNRILGNGIEIHTLQDGLIYTKDALADDLGISIIQ
RVKAYIAHQESKQKSFRVSQKWEQRAKLALAGEQRLTKMVPGWIDPDTFKLNEHAETVRLIFKLLLDG
ESLHNIARHLQSNGIKSFSRRKDANGFSVHSVRTILRSETTIGTLPASQRNDRPAIPNYYEAAIDAAT
FNKAQEVLDKNRKGRIPASDNPLTINIFKGLFRCQCGASVHPTGTKNKYAGVYRCNNHLDGRCDVPPL
KRKPFDKWVLENFLGMIDVGNDGESERKIAALQHEVEIVTARIKKATALLLEMDDIDELKIQLKELNQ
KRTELQTTIDSMRRKASLTDKELPQLKDIDLTTKAGRVECQLILSKHLKGLTLGKDSVTVTLQNDTEI
TIPTNPLPLNDGSPIFEIADKELLDIDAYQL
Ec04 integrase
SEQ ID NO: 103
MGKLLVTYIRWSTKEQDSGDSLRRQTNLIDAFYSKHKNDYYLLPAHRYVDKGKSGFHQQHKAQGSDER
RMFENVMSGAIPEGSLIVVENFDRFSRADIDTAIDDVRQILRKGVSILTLGDGELYDKSALTDPVKLI
KHIIIAERAHQESLVKQKRIAQVWNHKTQLARELKKPMGKQAPGWLELSEDGSHYIVDEDKASLVNII
YDKRLSGMSMFAICKWLNEQGYPTINQRKVRISKTKKPDGNWSALSVKHILTSRSVLGYLPAKISTED
RKTVLREEIEGFYPQIVTDSKFYAVQQLLEETGKGKTSSGEHWLYVNILKGLIRCKCGLVMTPTGIRK
PVYQGTYRCNGNKESRCSYGTVSRKLLDTQLCSRLFSKLSQLHDEATDTAKLDELQRRLNTVDSELEK
LTETLIQLPNITQIQEALRVKQEEKDELIVQLSREKARVKSVSSLDLSGLDMESVEGRTEAQIIIKRL
VKEIVVSGNEKLVDIYLHNGNMIRGFPLDGKDDHTLTLEEATDEMQSLDDMLIFGEPVTRIYPAGDME
EVDA
Ec05 integrase
SEQ ID NO: 104
MGKQNGKAYSYIRFSSKKQEQGDSVRRQIALAEHYAHANNLILSDKNFQDLGISAFKEGNRPSLGDML
EAIEQGQIEQGSTIIIESLDRLSRRGIDVTQQIIKSILQKNVFIASLVDGLLLNRDSVNDLVSVIRIA
LAADLAYKESEKKSKRLRETKGQQRQRALKGEVINKILPFWLERKQNKYIFSNRLATVKRIIELRQKG
LGTNKIAKILNEEGHKPLRSKGWNHTTIGKTINSVALYGAYQTSETTQDRKVILLDIVENYYPAVISK
EEFMLLQSDHKQNKPGYKSEHNAFAGLLKHECGGALVRKFHVASGKTYQYHVCANARDGKCNVTKNEK
NIEVALYQIMKELKLEKKTSFDKTLLEERNSVKTKIQELNNMLLELPKVPLSVLQTINNLEEKLQELE
EKIKHQDNIIASEKTFNINTLRETKDPQQLNMMLKRVIENIIVFNIEKRWRIKILYRNKHSQSFIWDG
SNITFVSDTKKLLELVKHTPEESK
Ec06 integrase
SEQ ID NO: 105
MGRGVITYLRYSSAIQGAEGADSTRRQNDLFKQWLKKNSDAQVVASENDEGLSGYKGKHLSGQFGDML
SRIESGEFPEGTILLVEAIDRIGRLEHLETEALMNRIIAHGIEIHTLQDGLIYTRDALSDDLGISIIQ
RVKSYIAHQESKQKSFRVSQKWEQRAKLALAGEQRLTRNVPGWIDPETFKLNEHAETVRLIFKLLLDG
ESLHNIARHLQANNISSFSRRKDANGFSVHSVRTILRSETTIGTLPASQRNDRPAILNYYEAAIDAST
FNKAQEILSKNRKGRTPASDNPLTINIFKGLFRCQCGASVHPTGTKNKYAGVYRCNNHLDGRCDVPPL
KRKPFDKWMIDNFLGMIDVVSDGESERKIAALQHEVETVTARIKKATALLLEMDDITELKAQVKELNQ
KRTELQTTIDTLKRKTSLTDKELPQLKDIDLTTKVGRVECQLILSKHLKGLTLGKDSVTVTLQNDTEI
IIPTDPLPLNDGTSILEIAEKELLGIDVYQL
Ec07 integrase
SEQ ID NO: 106
MGRRAISYIRFSSERQLKGDSVRRQSKLVTDWLDKNPEFYLDSSLSFKDLGKSAFSGKHLKGGLGDFL
TAIEKGLVKAGDTLLIESLDRLSRQDIDIASELLRRILRAGVDIVTLSDGEHYTRESLKDPLSLIKSI
LIMQRAHEESLRKSERVQAAWNRKKELISEGIKVSRRCPAWLRLNDDRRTFTIIPDKVEVVKRAFDLR
LQGLSFWAITRTLNDEGHLSLNQYTPKQKGWSDTAVKKLLRNRAVIGCFTPAGREEVQGYYPAIISES
LFYRVQQLNTGQYGRASVSSNPLSVNLFRGIIKCSECGATIVLGGYALKRFGMYRCPNRSANRCSAKA
ISRKQTDTTILYMLALCDRFETENTDTIDSLKLQREDLQRKISKMAELAIELDDMTIITEKLRDMKNA
LSKLNHDIEEETKRIKAITTGSLKDIDRTTKEGMIETQLLIKGVLKEIVIDAAKRRCKVTFHNGKVID
LSITENPSEDVTEAIQSLSEVTERGLIDVDEVII
Ef01 integrase
SEQ ID NO: 107
MGRTGLYVRVSTAEQEKHGYSIKVQLEKLRAFASAKDYTVVKEYIDAAQSGAKLERPGLKQLIEDVEN
NALDCVLVYRLDRLSRSQKDTMYLIEDVFLKNSVAFVSLQESFDTTSSFGRAMIGMLSVFAQLERDNI
TERLFSGRAHRAKRGFHHGGGIIPFGYRYDVETGELKRFENESNEVKAMFEMIANGKSVSSVAKEFNT
YDTTIRRRIANSVYIGKIQFDGETFDGQHEPIISKELFDKANVRMNARASNLPFKRTYLLSGLIYCGK
CGERCSAYESRSKHNDKEYRRAYYRCNARTWKYKQKHGRTCEQPHIRVDELEQAVMEQVKRLPLKHKV
KKRAFDFKPVENKIATIDKQKERLLDLYLNEHLDNEMENKKSKELDKSRDKLAKQLERMRMQAADSVE
SYQWLDGIDWDALDKDTLREVLERIIERIVIRDKDVEIYFK
Ef02 integrase
SEQ ID NO: 108
MGKRVALYMRVSTEQQAKHGDSLREQKETLYEYIEQHKDLKVVNEYVDGGISGQKINRDEFQKLLQDV
KENKIDLILFTKLDRWFRNLRHYLNTQEILEKHNVSWNAVSQQYYDTTTAYGRTFIAQVMSFAELEAQ
IDSERIKAVMANKIAQGEVVSGKTPLGYSIENKKLVINDDAPIVIDIFNYFLSSGSLRKTVYYLGSQY
GIVRDYQSVKNMLINKKYIGELRNNKNYCPPIIDKKLFYAVQKALPKNLKTNAKRDYIFKGLLKCSDC
QGSVAGQTIKARYKKKDGTESIYERTCYRCVKRRNNKLRCTNKRAFYEKNLERYIFEATKQKFEQIQI
NYSKKQPKIIKKKNSKKNIENKLDRLKKAYLNEVIDLEEYKKDREALMKELNEIEVEPAKIDIKNVEF
ILSKEFDEIYKESSEEEKNALWRSIIDNIIVFPDGNITVNFLI
Kp01 integrase
SEQ ID NO: 109
MGLRPICYERVSSIQQIEGGGGLDDQRSALEGYLDRNSDKFSNDRIFIQDRGVSAFKNSNISSESQLG
IFLQDVQNRKYGEGDALIVMSLDRISRRSSWAEDTIRFIVNSGIEVHDISASTVLRKDDPHSKLIMEL
IQMRSHNESLMKSVRAKAAWDRKIIEAVQNGTVISNKMPMWLKNVDNRYQVIQEKADLIIRCFEWYRD
GFSTGEIVKRIADPKWQMVTVSRLVRDRRLLGEHKRYNDEIIHNVYPKVIDDDLFLTANRMMDRVMLE
KNKPAEDLLLESDVVQEIFQLYESGLGSGAIVKRLPKGWSTVNVLRVLRDKNVVTQKIIDNLTFERVN
QKLSMNGVANRIRKDITIAQDDYITNLFPKILKCGCCGGNIAIHYNHVRTKYVICRNREERKICDAKS
IQYIRIEKNILKCVKNVDFQKLMIESTGSETSVLDGLREELSSLRREESSYNDKINERKLAGKRVGIH
LNDGLTEVQDRIEEIEKEIISAQTVREIPKFDFDMDEVLDPMNIELRAKVRKQLRLVLKAVKYWMEDK
RIFIQLEYFNDVLSHMLVIDNKRGGGEVMYEMSIEEKKGERIYTVHENGYAVFIASVTIGTELWSLAL
SRTRTIDSVGNYLSLLAREGFEIFVNEDQIDWF
Kp03 integrase
SEQ ID NO: 110
MGRQVITYLRFSSKPQERGDSIRRQKGLFERWLKDNPDAKVVDEFSDEGASAYHGHHLKGDFGRMLQN
IQDGKYLSGQTVLLVESETRLNRQKARNTENLVDLITGKGVDVICLESGKIYTSTNIDDLDTSIQLKI
AAHIAHQQSKEKSIKVSAAWEHRAQLALEGKQQLTKNVPGWIDPDTRKLNEHSSTVVTIFDLLLSGES
LHNIARYLQANNIKSFSRREKANGFSVHSVRTILRSESTVGTLAASKRNDRPAIPNYYEPAIDVATFN
KAQEILSKNRVGRAPASDNPITINLFKGIIRCQCGASVHPTGVKATYQGVYRCNNVPDGRCNVPTIKR
KPFDKWMLDNIVGFLERDDGNNTDKRKAEIEYQISLVTSKLKKATTLLLELDDVTELKEQVKELNIQR
SNLQSELDELNQRETLSDKPLHHLSEIDLTTKAGRVEAQLILSKFVQSIELQREMIIITLRNGTVIGK
SRDLSPVLSQDLMKQVVSSPSPTDIDMFSVITSDEEFRKSGKQVTKRS
Kp04 integrase
SEQ ID NO: 111
MGPKAISYIRFSTKIQSVGDSTKRQSKYINDWLKRNPDYYLDESLRFQDLGISGFSGANAKSGAFGEF
LAAVESGYIEAGSVLLVESLDRVSRQDIDTAGEQLRKILRSGVEVVTLVDNEWYTKESLKDSLSMIKA
MLVMERAHEESAMKSTRLRSVWAAKRERAAKGEIMSKRCAAWLKVSEDRSRFEFIPENVKAVQRVFQL
RLEGLSHVKIAKQMNDEGFSTLNQFKSVTGGWSQSSVTELLSNRSVIGFKVPSKSMAVKGVSEIPNYY
PSIITDEQFYSVQQLKQGSGRKPSSDLPLLTNLFKGVLRCSECGFIVVVAGVSAKRSGIYKCSMKSEG
RCNSVGFSRLQTDRALVQGLLYNTNRLSLNRDNGSAIGTLQSELEQLQKQRERLIKLAMLADDTESMA
KDLKALNSQIKDAEKAVSEVHQREQSSQLETISHLDLTVKKDRIESQIIIKRIVKEIRLNTAGKKCDV
FLHNGLKLYNFPLDRVVDGAQWLEILPLIDGDEFDFEGFTTKPRHIALEEAPEWVKEMEEQPKQ
Kp05 integrase
SEQ ID NO: 112
MGKKIIPYGYLRVSSLEQVRSGGGLEAQDEEVRRYITQKSDVFDIDKMVMMSDEGLSAYSGRNIKEGE
LGRFLADVDAGLIPAGSALVCYSVDRLSRQNPWVGTQLISTLIGAGIEIHSVAENQILRSDDPVGAIM
STIYLMRANNESVIKSERAKHGYTKRLNESIANNKVLTRQMPRWLYDNDGKYAIDPNMQKVIDFTFDS
YIAGQSTGYIAKKLNDMGLKYGDTSWRGSYVAKLIRDERLIGKHIRYSKQIKGVKREIIETIPDFYPV
TIDTDKFHIANNMLTSVAKNIRGRTRMTYGDISILRNLFNGVIKCGVCSGETSVVQNTRRKITNGVVT
YVPYKTFLRCRNRYELKTCTQGDIRYEVIERAILNHLMGLDITTLLAAPVDNKIERYRTELELCKAEE
EELQAIIKERENEGKRPRPQTLKSYEDVADRIDELTQLIESHVEDNFIPHENVDLDSITDVSNVSERS
LIKKGIATIADSICYKRISDFILVEIKYRNLNDKHVLVIDNKNSEMVVNFSIEYNEENKVYICNSFVM
EYDNLSCEFTVSKTTMEDYAHMMNFVDYVSDDESYNAKEFLVKNFTHIKFIDKSNE
PA1 integrase
SEQ ID NO: 113
MGPSAFSYVRFSSGKQAKGSSEHRQRAMLGQWLEQHPSFTLSDLRFEDLGRSGFSGEHLDHGLGQLLA
AIDSGAIKSGDVILVEAVDRIGRLEPLEMLPLFSRIVKAGVSVITLEDGHVYDRSSVNETSLFLLVAK
IQQAHEYSNRLSRRINASYTARREKAKAGLGIKRETPVWLTTDGQLVPHVAPHIAQAFQDYADGLGER
RICRKLRESGLEEFSKTNATTVRRWLKNRTAIGYWNDIPDVYPHVVDPALFYQVQQRLDAPKVDRAKP
SAHYLTGLVKCAVCGRNYNYKQRKHTDPAMLCTSRARLAGEGCSNSKTYPVIVLDQVRKLTSLPFLQH
AMESASSQADPSSQRLAVIDGEIGELSRKISEATKALLVLGFTPEIQESLEQLKTAREALEEERATLL
LPQAEKLTTAQLEAFSNGLLDDEPMKLNHVLQTAGYSMVVHPDGSIDVDGKRFVYEGASRKEKVYKLR
LIGEDKQWSLPILTPQMATYKSLFMAAVRLPGDPSEEELRRFEEAKHSER
PA3 integrase
SEQ ID NO: 114
MGPTAYAYIRYSSKAQGEAGRDSVDRQMASIQAITKQQGIELRTENIFSDTGISGFDGSNKNKGKLKD
LIDLIISQKIQDGDFVEVESIDRLSRQKMRLSKDLVYTILDRGVTLVTTIDGQMYSRAKDGMEQDIML
SVIAQRAHEESKIKSVRRKSAWNRAKKLADEEKEIFNGHNPPYGISENKEESRFEIVEEEAQEIRDIF
ESLKYVGVSLTIKKVNEYSKRKWTNRNIKHLLDTKYVLGSYMAQRRDENKKKVFERYIENYYPQIVSF
ELFNEAVASMKNRAHRKHYGNQTVGSLNIFRHSIKCSNCNASMLFEKQTNPKGVVYPYFQCFTRKELK
NGCDQPRFRFDLAFGVFLELVKFATTSSETINPNDWNNEITSVGSFHKTLFKLLTSTEKDKELEKKIS
HVKNLLLEQKNYQDNINKSFEAFTDGIIPAAFIKKASETEIKIEALERELAELNIESSTRNVSLLVHS
YNDIIDLYKTEAGRLKINSFFTSNNIAFSFSFDQKTRMLRCKVYYKDIHVDVINKKFPLHNPLKEFGI
DNLNQYFN
SA1 integrase
SEQ ID NO: 115
MGKVAIYTRVSTLEQKEKGHSIEEQERKLRAYSDINDWKIHKVYTDAGYSGAKKDRPALQEMLNEIDN
FDLVLVYKLDRLTRSVKDLLEILELFENKNVLFRSATEVYDTTSAMGRLFVTLVGAMAEWERTTIQER
TAMGRRASARKGLAKTVPPFYYDRVNDKFVPNEYKKVLRFAVEEAKKGTSLREITIKLNNSKYKAPLG
KNWHRSVIGNALTSPVARGHLVFGDIFVENTHEAIISEEEYEEIKLRISEKTNSTIVKHNAIFRSKLL
CPNCNQKLTLNTVKHTPKNKEVWYSKLYFCSNCKNTKNKNACNIDEGEVLKQFYNYLKQFDLTSYKIE
NQPKEIEDVGIDIEKLRKERARCQTLFIEGMMDKDEAFPIISRIDKEIHEYEKRKDNDKGKTFNYEKI
KNFKYSLLNGWELMEDELKTEFIKMAIKNIHFEYVKGIKGKRQNSLKITGIEFY
SA2 integrase
SEQ ID NO: 116
MGKVAIYTRVSSAEQANEGYSIHEQKRKLISFCEVNDWDRYEVFSDPGVSGGSMKRPSLQKLFDRLEE
FDLVLVYKLDRLTRNVRDLLEMLEVFEKNNIAFKSATELFDTTSAIGKLFITMVGAMAEWERETIRER
SLIGARAAVRSGKYIKVQPFCYDLVDQKLKPNQYAEYIRFIVDKLLSGKSANEVVRLLESKKKPPGIT
KWNRKTVLGWMRNPILRGHTKHGDLLIKNTHEPIISEDEHSKMLDIIDKRTHKSKTKHNSIFRGVIEC
PQCQNKLYLVSSIQKRANGGSYEVRRYTCATCHKNKEVKDVSFNESEIEREFINTLLKKGTDNEMVNI
PKPKDYDIENNKEKILEQRANYTRAWSLGYIKDEEYFVLMDETDKLLKDIEEKESPRINIELNEQQIR
SVKNLLIKGFKMATAENKEELITSTVDLIKIDFIPRRLNKEGNINTVKINEIHFKY
Pf13 integrase
SEQ ID NO: 117
MPKAISYIRFSTGRQSLGSSHERQRQAVTRWLEKHPDYTLYDKPYDDLGRSGYSGDHVDNAFGHLLAA
IEDGTIPKGSTILIEQIDRVGRMEPFEMFPLLSRIVNAGVDLVTLDDGITYNRQSVNNNHLFLLVAKV
QAAWGYSKTLSERTKASYAIRREKAKNGEPIKRFAVAWLTTDGKLKSHLVPYVKQVEDLYISGVGKNT
IANRLRASGMPELASISSPTIDAWLQNKTAIGFWNDIPGVYKPVVTPEVEMQAQKRRQEVKTQSRSRT
SKNFLVGMVKCGVCNANYIIHNKEGKPNNMRCLTHHRLKDAGCTNSETIPYQVVHFIYLSSAPSWIDK
AMKVIQLTDNEKRKLYLVTEREELTASILRMAKLLARTDSPELESEFDLANERRASIDIELSVLDRKA
DNGVESKSTSIFVGYEATLEHDRLAFHDPIQLSALLKQAGYSITVQPGRKLYVAESNVPWVYTGVARK
GNTALGYRIQDGEMEYTISNVIPEAVDVQAYKNNPDGEMQHVADRSYKHVKSPTLLNPTGLRNTNVMT
IEKFESANAAMQRLTSGV
Td08 integrase
SEQ ID NO: 118
MKAVAYIRISSSEQEYKRQHEELSELARFKNLNLVKVFADVVSGSKTKAKERASFEIMDKYLLDNSDV
KNLLILETSRLGRKKLDVLNTIEDFFLRGVNIHIKDLNLCTMENGKRSITTDILVSLLSIMADEETRL
LSERIKSGKMSKAKENKAFGAKVIGYKKGKDGTPIIDEKEAPIIKRIFELASQGLGMRKISSIIESEF
NREFAIGTLSSIIKNSFHKGKRKYKDLILDVPPIVSKGLWQKANDSINSRSKFGSRKYVNTNVVQGKI
KCGVCNSVMYQKVIPKGRINSFVCKDTKCKNSINRPWLFRMIRLIVDKHALKNKDEKVREKIKLQITS
HKAELQINNKLLAKLKRRSEKIRILWLDDEITDARYKSDISNVNKEIKLCNTKSKEIEKAIVIAEKSL
KNDIEHYSKELSVFKTEIQDVLSHVIIDKERVLINIFGWREYDLSKPNSIKLGWEARKPISERYKNEK
LPLRKPISDEDLNLMIDNYTL
Se37 integrase
SEQ ID NO: 119
MNKVAIYVRVSTTNQAEEGYSIEEQIDKLKAYCMIKDWSVYDIYVDAGFSGSNIKRPAIQKLIKDTKR
KVFDTVLVYKLDRLSRSQKDTLYLIEDVFLENKIDFVSLLENFDTSTAFGKAMVGILSVFAQLDREQI
KERMQLGKLGRAKSGKPMMWAKVAYGYTYHIGTGKMTVNQSEAIIVKEVESSYLNGRSITKLRDDLNE
KYPKTPAWSYRTIRQMLDNPVYCGYNKYKGQVYPGNHAPIISKEIYNQVQDELKIRQQKAYEHNNNYR
PFQSKYMLSGIAQCGYCKAPLKITLGTIRKDGTRFKRYQCVQRTPRKTKGATVYNNNEKCNSGFYEKD
DIEAYVLESISKLQTDSNCIDELENDEPEKLDRDALNKEIETLSNKISRLNDLYINNLITLDDLKTKT
DTLQSKIDILKEKLEKDPALERQKNKQKMLKKLDTKDIFKMDYEEQKMLVRALINKVQVTADSIKILW
KI
Ct03 integrase
SEQ ID NO: 120
MENVCIYLRKSRADEEMEKTLGHGETLSKHRKALLKIAKEKNLHIVEIKEEIVSGESLFFRPKMLELL
KEVEDKKYNGVLVMDMQRLGRGDMKEQGIILETFKNAKTKIITPNKIYDLNNDFDEEYSEFEAFMSRK
ELKMITRRMQGGRVRSVEDGNYIASAPPYGYDIDYILKSRTLKINEHEAEGVKIIFDSYINGNGASAI
SEKLNNLGFKTKLGNNFSPSSVLTIIKNPVYIGKVTWKKKEIKKSKTLGKVKDTRTRDKSEWIIANGK
HKPIISEEVWNNTQEVLKNKYHIPYQLTNAPINPLAGLLVCGVCGKAMVMRPSRGILRVMCVHKCGNK
SVRFDYVEKAIIDSLEQYYSNKKLEVKKQKTIQNTSNEEKELILLENELSTLNKQRLSLEDFLERGIY
TEDVFLERSKNIDSRINLVESEMKKISEKIKFKKTKKDTKALLQTLNNAIENYKSSSDVITKNSYLKS
ILNDITYIKTPEQKRNSFSITLNPKLRF
Ps40 integrase
SEQ ID NO: 121
MIAAIYSRKSKFTGKGESIENQIQLCMDYAKNLGINEFLVYEDEGFSGKSMDRPKFKEMLKDAKDKKF
DYLICYRLDRISRNISDESTLIEDLNKLNISFVSIKEQFDTSTPMGRAMMYISSVFAQLERETIAERV
RDNMYELARTGRWLGGMPPYGFISTQINYYDENMNQRKMYKLKVDEDTIEIVKLIFDKYLELRSLSKL
YKYMYENGIKGTRGGNLDPSALSLILKNPAYVKADKSVVDYLRKSNIDVMGDIDNIHGILTYAKNTDS
PIAAVAKHKGVIDSDKWIEAQRLLNANKAKAPRAGTGSKALLSGLLKCSKCGSNMRITYKNSKSGTIY
YYICGTKKSLGVSACDCRNIRSDKAESKVIDELKNKSIKSIMSSYKDSKLENSKNIKNIKTEINSINS
QIKEKETYIDNLVMQLAKVTESSASTFIINKLESLNNDLSNLKSQLESINTISMENKQVDININMLID
NLNKFNKEIDNSDINKKRLLLSTVVDYMTWDSDTDTIKVNLIGINPSNTIASGK
Sa10 integrase
SEQ ID NO: 122
MKVAIYTRVSTLEQKEKGHSIEEQERKLRAYSDINDWKIHKVYTDAGYSGAKKDRPALQEMLNEIDNF
DLVLVYKLDRLTRSVKDLLEILELFENKNVLFRSATEVYDTTSAMGRLFVTLVGAMAEWERTTIQERT
AMGRRASARKGLAKTVPPFYYDRVNDKFVPNEYKKVLRFAVEEAKKGTSLREITIKLNNSKYKAPLGK
NWHRSVIGNALTSPVARGHLVFGDIFVENTHEAIISEEEYEEIKLRISEKINSTIVKHNAIFRSKLLC
PNCNQKLTLNTVKHTPKNKEVWYSKLYFCSNCKNTKNKNACNIDEGEVLKHFYNYLKQFDLTSYKIEN
QPKEIEDVGIDIEKLRKERARCQTLFIEGMMDKDEAFPIISRIDKEIHEYEKRKDNDKGKTENYEKIK
NFKYSLLNGWELMEDELKTEFIKMAIKNIHFEYVKGIKGKRQNSLKITGIEFY
Td01 integrase
SEQ ID NO: 123
MLAIYARTSTDKAENSTIEQQVKAGIEFASKNNMNPKVFQDKGVSGYKIEEDENKNPFENRPAFTQMI
EDIKKGTIDAVWVWEHSRISRNQYASAYIFNIFSKYKIRLYEKDKEYDLNDPNTQLLRTMLDAVAQYE
RQLIVKRTTRGLHNAIDNGKRSYPSLLGYRKTIKNSKGNYIWEPVESELLQVKNWFTRYKNGESLKNI
VFSQNSNENKASHILKRTTHLSRTLQHYVYTGYSLNTKGLDYLKKEDNFEIDNLQMLHNPDYWVKSIP
YSLEIINREDWIEVKERLRIYKEKHKKNTNRRAEKSIGTGLITCGYCGAKFFYQVQAHKRKKGLVLYP
YYFHMSCLDRTCLQSPKSVSQDKIDTIFKIFVLYSTITSDSKSKFLKERLFQEDIEVKAIKEKVKILK
RDHQKTETQISKFKTALETTEDVGAITVLAKQIDNTETTLTEIKNSIISGEAELQERQEAMNKTRSQL
MHYSICDLLTQFFEKWNIEEQRNHLLKIVDNAVITGTTLNIKSGEYTYIFDTNKKYEFPTVVYNEMLK
EAKEDIDYSSFFRNKPDDHFERRMWSILVMSESVWHICEWRDKEKQLIF
Enc3 integrase
SEQ ID NO: 124
MMKKIAVLYMRMSTDMQEHSIESQERVLMEYAKRNGYVVIRKYIDRGISGQHASKRPDFLKMIDDSET
GEFQFVLIYDSSRFARNLVESLTYKSILKENCVRLISMTEPNLEDDEMSLYIDAMQGATNEIYVRKLS
KSVKRGHNDRALRGDLPGDVQFGLKRLKDGSIVLDEVKAPIMRWMYEAIYYDDSSYYSICETLAAKGI
KSQRGNIIDSRQVKRMLMNIKNKAYHWAEKDGKPILKKGNYPAIVDEEIFDAVQEIIAERAKHYKKNE
KPAEFRKHWLSGLLVCPHCGAGYSYNTRKPPQHDAFRCGNQTRGACKKGSSILVDVAVEMVLDKLSEV
YAGPLAPYVKNITVSQPEPQIDYDKEIKLLEAQLKRAKQAYLAEIDTIDEYAQNKRRIASNIKELQEA
KNQAQEGAALNEPQFKVKLLNAITLLKSDCPMSEKIPAARSIIEKILVDPRNKTMDIYFFA
Fp10 integrase
SEQ ID NO: 125
MTENNNRVCCLYRVSTDKQVDFNSNHEADLPMQRKACHKFAESKGWVIVHEEQEEGVSGHKVRAAARD
KLQIIKDYARRGKFDILLVFMFDRIGRIADETPFVVEWFVRNGIRVWSTQEGEQRFDNHTDKLLNYIR
FWQADGESEKTSVRTRTSLRQLVEEGHFKGGNAPYGYDLVRSGRINKRKHELYELHINEQEAAVVRIV
FDKYVYEGYGPQHIATYLNNSGYRARSGKCWHPSSIRGMVQNLTYTGVLRCGDARSELMPDLQIVPQE
QFENAQRIRNERSVRSTAEAENRLPLNIHGKSLLAGNAYCGHCGAKLELTSSRKWRKMADGSLDDTLR
IRYTCYGKLRKQTNCTGQTGYTVHILDEIIDKAVRQIFSKMRGIPKEQIVTKRYEKENTERKNHLQDL
QTQRNKAEKDLLALKTEVLACIKGESVLPRGTLAEMITEQEEKLAELENLCESATEELEKTAELMDKV
SRLYDELISYADLYDSANFEAKKMIVNQLIRRVDVYRGYQINISFNFDLTPYIEGE
Ph43 integrase
SEQ ID NO: 126
MKIAYARVSSREQSENSHALEQQIARLESSQVDRVIQDVESGSKNSKSPGFRELMDLVKEGKVAEIVV
TRLDRLTRSLSSLQKTMEILKAHGVALVSLDDSIDTSTAAGVFHLNMLGALAEMEVGRLSERVRHGWS
HLRDRRVAMNPPFGYRKENDQHVLDTTPFLCLISDRSEWSRAKISRYYIDTFLQERSIRLTLRVVYPH
FGIQVYRSRRRGPHATRLIRFSPSAFNEWLINPVLQGHLSYRRNASGNRKREDPKTWQIIPNTHEPLI
TAEEAAMIKQILSRNRQAKGFGSPKRRHSVSGLVFCGECRSACYHQSGCQNYARSKRLGIPQIIRRYY
QCKNWRSRACPQKAMVSLDIIEEAVIAALISRAEDLAKMADTPAPTPEPLALRELRSQLADLNRMAYN
PAIENAKAQIKNQIAGMELDLTHTTQESSRLGQLISALADPDFWKEGLEPNEKSQLFQDLVSQVIIKD
GAVLEVKLKV
Sm18 integrase
SEQ ID NO: 127
MITTNKVAIYVRVSTTSQAEEGYSIEEQRDKLEAYCKIKDWSVYDVYTDGGFSGSNTNRPAIERLIKD
AKNKKFDTILVYKLDRLSRSQKDTLYLIEDIFIKNNIAFLSLQENFDTSTPFGKAMIGLLSVFAQLER
EQIKERMQLGKLGRAKSGKSMMWAKTSYGYNYHKETGTVTINPAQALAVKFIFKSYLAGRSITKLRDD
LNEKFPKEIAWNYRAVRNILDNPVYCGYNQYLGEIYKGNHESIISKEDYDKTQNELKIRQRTAAENVN
PRPFQAKYILSGIGQCGYCGAPLKIMLGVKRKDGSRLKKYQCHQRHPRTLRGITTYNDNKKCDSGFYY
KDDLETYVLTEISKLQNDTNYLEQIFSEDNTETIDRDSYQKQIDELSKKLGRINDLYIDDRITLEELQ
TKSAEFTSMRSSLETKLGNDPALKQKDRKKGMINILNQRDILTMNYEEQKVVVRSLIDKVQVTAEDIV
IKWKI
Pf80 integrase
SEQ ID NO: 128
MKQAISYVRFSSDRQRHGSSVERQEGMIADWLKRHPDYEMSDLKFSDLGKSGYHGEHIKEAGGFGKLL
KALEDGFIRAGDVVLVEAIDRTGRLEPMDMLTLVINPILKAGVSIITLDDNTTYSKESVNTAQIFLLV
AKIQAAHGYSAALSTRVADSYKKRRKDAAKYGIVPRRITPVWLNPDGTVREDVAPWIKTAFELYVSGV
GKSTIAKRMRESGVERLAKASGPGVEGWLRNKAVIGKWETLEGTPDHQIIDDIYPAIIEPSLFYKAQV
HAEKMKTQRPIKTASHFLVGLVRCGECGKNYIVQNLHGKPHSMRCRTRQSQNECTNSHCVPKPVLDAI
YRYTSVTAAIEAVQQQQMGVNDKEIVTREAELLAITKRVDGLVQALTQTGPIPEVIEALKQSRIEREA
AESALVILRSTVVPSAGTHWQEMGKVWKLEAADAQRLAAMLKLVDYHITVAPTGEITASHSEVLYRYI
GVDRALDKYKLLANGKLMLIPKGYVDDFPYHEPFQEMQSENTWDEADYDNLRQQHQ
Bs46 integrase
SEQ ID NO: 129
MEDSSNKSVGIYVRVSTDEQAKEGFSISAQKEKLKAYCVSQGWANFKFYVDEGKSAKDTHRPSLELLL
RHIEQGIIDTVLVYRLDRLTRSVRDLYTLLDYFDKYNAVERSATEVYDTGSATGRLFITLVAAMAQWE
RENLGERVKMGQNEKARQGQFSAPAPFGFIKEGKSLVKNHEQGEILLEIIDKVKKGYSTRQIANYLDD
SGLLPIRGYRWHPGTILTLLKNPILYGSFRWGDEIIEDTHEGYISKDEFDRIQEILKERSIVKKRDSY
SVFIFQSKIVCAGCGNRLASERSKYFRKKDKQYVETNNYRCQTCAQNRKPSIMGSEKKFQKALVKYMQ
NVTPKLEPKIPEEKKHDYEKVHQKILNLEKQRKKYQKAWSLDLMTDEEFEQLMYETKEALKSAQNELA
AAHSSDSQNSQIDIERAKEIVKMFNENWSVLTNEEKRSIVQELIKHINFTKEDGEIIITHIEFY
Pf48 integrase
SEQ ID NO: 130
MPTAFSYARFSSATQKKGSSLERQRAMVARWLVAHPDYSLSDQTFQDLGKSGWKGEHIKEGGGFAELL
VAVQAGLIQKGDAVLVEAIDRTGRLPVLDMLSIIVSPILRAGVSIVTLEDNLVFTEASLNEGGHIYIL
VGKIQAARQYSDNLSRRLTASYDSRRRLAKEGKAPKRNTPVWLTSNGDVIGEVAEQIRLAFELYTSGL
GKAVIAKRMRESGVPALAKTSGPGVEGWLRNEAAIGRWNGSEVYEPIVDLSRYQLAQIEGERRKTTPT
AKTATHFYVGLVKCGSCGGNFIMRTIKGVQVSMRCRKRQELKGCDNKKVIPKVVIDAVYRHTSTPAAR
KLVAHERRSVNEKAIIAAEAKVLELAKEIEAMVLAFSGAMAIPEVVGRIQALHAEREAAERELALLKV
TVERPPTDWRVQGRVDDLGRTDPQRLAAMLRSVGYTITIDSDGRLCTSESKTVYRYAGVNRREDMYRL
AVAGGKELLISKIPVEVEDEWWEAEDGDEVVTSEWDADNPDAMRSRHG
Rb27 integrase
SEQ ID NO: 131
MQEHSPSNHSSGPVRAYSYVRMSTHKQLRGDSLRRQLERSKAFADEHSLLLDDSLRDIGISAWKGRNF
KTGALGRFLSMVESGEIPKGSYLLIESLDRLSREAVPDALTLFMAIINAGITIATLGDDRQIYSRDIL
NGDWTKLIIGLAVMSRGHEESQTKSERVRAAIHRKRENAREGKGQITGLTPAWIDAERIGANRYTFTL
NHHTETVRAIYEMAARGLGATVIARKLNAEGVPAFKSKDGWYQSIIKALLSRHDVIGTFQPHRVQDGK
RVPDGDPIESYFPAAIDKDLFLRVQSMRSNPGRPGRKGDMFTNLFTGLCHCSHCGGPMTMKLSRVKGN
ENGRYLVCSNYVRGHRCTEGNRHFRYEPFETAILDHVRELNLAEAIATTMTNEAITGINETIAALTLQ
LDELRRKEQRLAMALEDDNQPIDSIIDLLRQRQQERHAIEAGLQYHQQERHRLTVRHNDPAQTCDRIG
QLRTAWEQADEATRYGLRSEAHAAIRELITEISFDSGSHSAIVIVANGISAYRLQDGLINGRFHAFAA
SA1 integrase
SEQ ID NO: 132
MKGKIALYSRVSTSEQSEHGYSIHEQEQVLIKEVVNNYPGYDYETYIDSGISGKNIEGRPAMKRLLQD
VKDNKIEMVLSWKLNRISRSMRDVENIIHEFKEHGVGYKSISENIDTSNASGEVLVTMFGLIGSIERS
TLVSNVKMSMNAKARSGEAITGRVLGYKLSLNPLTQKNDLVIDENEANIVREIFGLYLNHNKGLKAIT
TILNQKGYRTINQKPFSVFGVKYILNNPVYKGFVRENNHQNWAVQRRSGKSDENDVILVKGKHEAIIS
EDVFDQVHEKLASKSFKPGRPIGGDFYLRSLIKCPECGNNMVCRRTYYKTKKSKERTIKRYYICSLEN
RSGSSACHSNAINAEVVERVINVHLNRILSQPDVIKQIASNVIEELKQKHSNQTEIKYDIDSLEKQKA
KLKTQQERLLELFLDDQMDSEMLKAKQSQMNEQLEMLDKQIKETQQARESQAEVPDFDKLKSRLTMMI
SRFSIYLRKATPEAKNQLMKMLIDSIEITTDKQVKLVRYKIDESLIPQSLKKDWGSFFMPKFQFEIDG
RKNYFIDQITTFTT
Bc30 integrase
SEQ ID NO: 133
MTVGIYIRVSTEEQARDGFSISAQREKLKAYCVAQDWDNEKFYVDEGVSAKDTNRPQLSILLNHIQQG
LITTVLVYRLDRLTRSVMDLYKLLDTFDKYNCAFKSATEVYDTSTAMGRMFITIVAALAQWERENLGE
RVRMGQLEKARQGEYSAKAPFGFDKNKHNKLVINEIESKVVLDMVRKIEEGYSIRQLAIHLDSYIKPI
RGYKWHIRTILDILSNNAMYGAIKWSNEIIEGAHEGILTKERFIQLQKILSSRQNIKKRQTHSIFIYQ
MKLICPNCGNRLSSERSRYYRKKDEQHVECNQYRCQSCALNKHTTKPFATSERKVESALMNYISNLQF
EQVPKINNENNELEILKKQVKKVEKQREKYQKAWSNDLMTDDEFTERMNETKILLNSAKKKLQTLEVN
NHQEIDVDVIKEKVNNIKKNWFHLSPDEKKQFMSMFIENIKIDKKDGVTEVLDIEFY
Cd04 integrase
SEQ ID NO: 134
MNNRIDAIYARQSVDKKDSISIESQIEFCKYELKGGNCKEYTDKGYSGKNTDRPKFQELVRDIKRGLI
AKVIVYKLDRISRSILDFANMMELFQQYDVEFVSSTEKFDTSTPMGRAMLNICIVFAQLERETIQKRV
TDAYYSRSQRGFKMGGKAPYGFHTEPIKMDGINTKKLVVNPDEAANIRLIFEMYAQPTTSYGDITRYF
AEQGILFYGKELIRPTLAQMLRNPVYVQADLDVYEFFKSQGTVIVNDAADFTGINGCYLYQGRDVKPS
KKNDLKDQMLVLAPHEGIVPADIWLACRKKLMNNMKIQSARKATHTWLAGKIKCGNCGYALMSIENPS
GRQYLRCTKRLDNKSCPGCGKIITSELEAVVYQQMVKKLEKHKTLTGRKKAAKANPKIAALQVELLHV
DSEIEKLVDSLTGANNVLLSYVNVKIAELDGRKQELVKQIAELTVETISPGQVNQISGYLDTWDDVSF
DDKRRVVDLMITTVAATSDSLNITWKI
Sa34 integrase
SEQ ID NO: 135
MNKVAIYVRVSTTNQADEGYSIDEQIDKLKAYCEIKDWVVYKVFTDAGFTGSNIDRPAMTNLISAAKK
RQFDTILVYKLDRLSRSQKDTLYLIEEIFIKNGIDFLSLSENFDTSSAFGKAMIGILSVFAQLEREQI
KERMMLGKVGRAKSGKTMMWAHPAYGYTYNKETSSLDIVPAEAALIKKIYELYIKGKSISKLRDYLND
NKIFVNKSVPWSHRTISYALTNPVYCGMIRYEGKLYDGLHEPIITKELFNKTQEVLAERRMEASKKNP
RPFQSKYMLSGIIRCGCCNAPMKSLLGMPRKDGTRTRRYQCINRFPRKTKPVTVYNDNKKCDSGYYYM
EDVEHAVLHRISTLYSDEIEASEFFEDEITFDIQKVKDEITKIESKINKINDLYINDFISLDSLKKQS
ANLINEKKIIENEIEKENSKQVNNLKEDALKILATNNIHDLDYEMQSYVVKSLIDKVFVTKEDMEILF
KK
Pp20 integrase
SEQ ID NO: 136
MLHCGFPCHEERAMPSAVPYIRFSSARQTTGSSAERQRQMVTQWLTQNPDYILSELTYEDLGRSGYHG
EHLNDDNGFAKLLQAVEAGSIKAGDVVLVEAIDRAGRLSPMQMLKRVIIPIIEAGVSIITLDDNVTYD
ESSVEGGHLFLLVAKIQAAHNYSKQLSDRTKASYAIRREQAKATGKVKRHTPIWLTSEGEVIEHVAVH
VRQAFELYVSGVGKTTIANRLRASGVPELATCSGPTVEAWLRNQAAIGNWEYGKDDPDKPSEIIRGVY
PAVVSDELFLQAQLRKKAAATKPRERTSKHFLVGLVKCGVCGSNYIIHNKGGKPNNMRCGTYHRLKKA
GCTNDETIPYQVAVYIYSETATHWVDKALQQVQLTVNDKRKLVLTTERDALTTSITNLTEKAAALNIP
ELWKKLEEESNRRKVVEDELAVLERTPDAGGESGFSAALSQDQMMIHDPIQLSALLKQVEYSIVVYPN
KLFTVSGEVYPWLYLGPKRKPKSNVTLGYRMLYLGDEIIISPDVPVTLDWGAPTDNPVEQMRYMLRRA
YKMVSAPKPYEYNDDVAE
Efs2 integrase
SEQ ID NO: 137
MSKRTRRTFSQEFKQQIVNLYLDGKPRVEIIREYELTASAFDKWVKQSKTSGSFKEKDNLTPEQKELL
ELRKRNQQLEMENDILKQAALIFGPKRQVIDANKHLYPISAMCRILGLSRQSYYYQSKPKKDESELEE
VVAEEFIRSRKAYGSRKIKKALSKRGIKISRRKISRIMKNRGLKSSYTVAYFKIHHSTCNEAKTTNVL
NRKFLRDNPLEAIVTDLTYVRVGKKWNYVCFNLDLFNREILGYSCGEHKDAVLVKKAFSRIKQPLTEV
EIFHTDRGKEFDNQTIDELLTTFDINRSLSHKGCPFDNAVAESTYKSLKVEFVYQYTFETLQQLDLEL
FDYVNWWNHLRLHGTLGYETPVGYRNQRLAQRILDNELGCANASEAV
Pf15 integrase
SEQ ID NO: 138
MRSAIPYIRFSSARQTTGSSAERQQQMVTQWLTEHPEYTLSDLTYKDFGKSGYHGEHVKDGGGFAKLL
AAVEAGDIKAGDVVLVEAIDRTGRLHPLDMLNKVITPILAAGVSIITLDDKVTYTHESAASGHLFLLV
AKIQAAYGYSKQLSERTKASYAIRLEQAKEGNKVKRNTPVWLHSDGRINDDVAPYIKQAFELYVSGVG
KTAIANRLRASGVPELVKCSGPTVEAWLRNQAAIGNWEYGKDDTDKPSQIILGVYPPVISNELFLQAQ
HRKSAVATKPRERTSKNFLVGIVKCGVCGANLIIHNKDGKPNNMRCLTHHRLKDAGCTNKETIPYQVV
HFVYLQTAPAWIDKAMKVIQLTDNEKRKLTLTTERNEVTASIQRLAKKIAKVDSVELEAEFDLVNERR
AAIDIELNILGRTDDDGAESKSKSNYVGYESNLEHDRLAFRDPIQLSALLKQAGYSIVVQPGRKLYLP
NDNHPWVYAGVVRKGNMTLGYRIRNSEEEFTISQAIPEVPDVRLYGNIPNGDLVHVAERSYKYAKPPT
LLNSSDKHSRKGVFVLRFESADIAMEYMKSGIETDSK
Ps45 integrase
SEQ ID NO: 139
MKQAISYIRFSSARQEGGSSVERQEGMIAKWLLDHQDYELSKLNYSDLGKSGFHGEHVKEGGNFGKLL
KAVMDGYIKRDDVVLVEAIDRTGRLPALQMLSDVIAPILRAGVSIITLDDNTTYTEASVGGPHLFMLV
AKIQAAHEYSRTLSRRVEDSYKKRRKDAKEKGVAPKRMTPVWLNSDGTIREDVAPWIKTAFELYVSGV
GKSTIAKRLRESGVERLAKASGPGVEGWLRNKAVIGKWETLIGTPEHHVIDDVYPLIIEPSLFYKAQV
HAEKMKTQRPIKTAKHFLVGLVHCGECGKNYIMQNLHGKPHSMRCRTRQSQNNCSNSYVVPKPVLDAI
YRYTSVTAALEAVQLQQLGVNEKEIVTREAELLTITKRVEGFVQAVNEAGPMPELLTALKQARIERES
AENALVILRATVVTPPANQWREMGKVWSLEAEDAQRLSAMLRQVGYNITVGKGAIIKSSHSDVVYQYL
GVDRVKDMYRVLADGEMKLIHKSQVDDYPYHEPFHEVVGEATMDETDKENLLLQYQSS
Sp56 integrase
SEQ ID NO: 140
MSTSIPEESGPNDLELRGTPAGLPPYADLVAANPNAIFVGAYSRISDDWRKNKSKKAAASRWSAGKGV
ANQHRRNDMNAGRHQVIVVHRYTDNDLVASRLDVFRPDFAQMLKDLKLGRTKDGYRLDGIICVHQDRL
QRTDTDWEHFVHALLAKPGRLLWTPSGSSDLTDEGEIVKTGIMAVLNKAESMKKKRRIRDWHQDLILD
GLPHSGPRPFGWNEDKMTLRAEEADYLAWAIRERIKGKALSTLCAEAKKRGLKGTKGGEIAPTTLSQM
MTAPRVCGYRANRGTLALDENGAPIVGKWDTICKPEEWEAVCATFSPGSTYMHRGPGAPRVTGKPKTV
KYLASQLLRCMNKVERDGETRICNGTLTGSPTKSARSPYTYRCGSCNKNSIAGPMVDRQITRLLLGKL
GEAQITYRRPELAWHLESTLKTLADRLAGLENRWMAGEVDDEQFYRLSPGLQAEVRKLRAERARWELE
NAAGSEEPADIIRKWRSGELDLAQRRRILFDVFAAIQVTPGQKGSKTPNPHRLKPIWG
Dn29 integrase
SEQ ID NO: 141
MERVLMHLRKSRADLEAEARGEGETLAKHRNILLKLAKERNLNIVKIREEVGISGESLIHRPEMLETL
KEVEEGLYDAVLCMDIDRLGRGNTKEQGIILEAFKNSNTKIITPLKTYDLNNEFDEEYAEFGAFMARR
EFKFITRRLQRGRVATVEEGNYIAPRPPYGYIIEKNNKERYLIPHPEQAPIVKMIFDWYTHDDPNVRM
GASKIANELNKFCKSPTGIAWKGSTVLSILKNAVYAGRIQWRKKEEKKSITPGKKKDVRMRPKEQWID
VEGKHEPLVSMETYLKAQEILERKYHVPYQIQNGITNPLAGLVKCGICGSSMVYRPYTHQRHPHLICY
NRYCTNKSSRFEYVESKIIQGLHQLLAQYKADWFKHQRPKVNDDSVDLRQKALHRLEKELNDLYKQKD
NLHNLLEKGVYSIDTFLERSNILAERIDDTKKAIHDAEKALAEEVQRNKVKKDIIPTVENVLELYYKT
QDPAKKNNLLKSVLDYAVYRKEKHQRDDDFTLVLYPKLPQINS
Vh73 integrase
SEQ ID NO: 142
MQDLPKQAYSYRRFSFLTQKFGSSLKRQTKLAQDYATQHGLTLSDTSFEDLGVSAYRAANASEDAGLG
QFLLALREGKITTPCTLLVENLDRLSRAKIKVAMRQLWDITDQDVHVVTLVDGRIYTKDMDFEDIMLA
GLIMQRAHEESETKSKRLQEKWQERRTLGKFIHKNCPFWLTPNKDRTDYEVNKYIETVHQIYAMALGG
LGSRVIAQELNSSGITAPRGGLWSTATITKVLNNRAVLGEYQPKQRVIVDGVRSEKPIGSPIQNYPVI
LDSDYFDQVQSALRGRHKGNNRNSTKTYRNVLKHIAHCGCCNGTIRLKQQQHLYYLQCSVECKGSRPV
SIRYLHDWLNEVWITSDFASVSLSDVPEAKELATLESELEKLTEAVSGLAAAYAATLDPTINSQLLET
SAKKVETQTKLDDLRSELSPYNQTKAAQFERQMLVDLAFSERNEVENFVARTKLTGLLAQLKDFIIHK
GQNDIVTFEIKTAQNESKTYTAVKNPYHKTRKLTGKIWNY
Em12 integrase
SEQ ID NO: 143
MKKRAGLYIRVSTEEQVDNYSIPEQKRRLEAYCQSHDWAIAEEYIDGGFSGAKLDRPAMQKMIADSKA
GNLDVVVSIKLDRLSRSQKDTLHLIEDIFLPYHVDYVSVNESFDTGTSFGRAMVGILSVFAQLEREQI
LERMHSGMEARAKTGLYHGSKPPYGYALEKGVLKINPTEALAVRKVFDLWLKGYSYNKISEIMEETYH
GEKAWMHPSAINQLLTNPAYTGKIRFAGEVIEGQHEPIIDEITFKKANLRLETRAANRGRQTTYLLTG
VIWCGNCGSRFGINMSTCNGVRYTYYTCSPNRRKKGNEGIRCCGNKPIPTKKLDPLIIEKIKRLAISK
DFFEEIQKPDASPSDAIASLESAAAEIDKRIGKLMELYSMDGIPTDTLSQQINTLYAQKKDLESQISE
KQSGFKQTYEDYKEVEDKVDYAFKSGTIEEQKSIVRSLIKRIDILNQEITITWNFQ
Pc64 integrase
SEQ ID NO: 144
MRVALYYRVSTKLQEDKYSLAAQKEELTKYAKSQGWNIVGEFKDVESGGKLEKKGLTALLDLVDEGGV
DVVLVVDQDRLSRLDTVAWEYLKSVLRDNNVKIAEPGNIIDLNNEDDEFISDIKNLIAKREKRAIVRR
MMRGKRQRMREGKGWGLPPFEYQYDRQTGKYKAKPEWKWVIPFIDNLYLNEQLGMKAISDRLNEISKT
PTGKSWNEHLVHTRLVSKAFHGVMEKTFANGETISIPGIYEPLRTEETYRKIQEEREKRGEQFSVSGR
KGSEKHILKRTKITCGECGRKIQIATHGTKEKPIYYAKHGRKERVDGSVCDININTVRFDKNIMTALK
EILSSEEQAKKYINLDIDQNELNALKKNIKTLNKRISKLQESLDRLLDLYLAGGLKKEKYLEKQKQLE
SQIEIYKKELDQNELKLKTVESNMWNYEYLYEYLEVIADLEKELTPLERAQLVGKIFPTATLYREKLI
LTADVKGIPVDIEIPIDPDPYPWHPKKRNTSIK
Vp82 integrase
SEQ ID NO: 145
MRKVYSYMRFSRPEQAKGTSIERQSNFAEQYALEHGLELDKSLTMMDKGLSAFHGVHKTKGAFGQFLA
AVTAGKVPSGSVLVVESLDRLSREDPLIAQAALTDLILSGITVVTAADNQVYSREEIQQNPFKLIMSL
VVMIRANEESETKQRRSNAFLKSALNQYQANGKIRRLGSDPSWLDENKDNDTYSFNERVEVIRRILNL
YNKGIGSLKIARQLTEESILTLSGKRSAWGQTTVANIVKSHALYGMKRINVQSVEYDLEDFYPALITK
SKWLALQQHRTKRSKSMHGRGEVVHLITGHGKGFTTCGYCGGGLGAQTQKQYKRSGEYSRTVTRLHCL
THKETSSCCSSVFLEPIESAVLFAACIGVEPQSLVPTVNNVNYSALIDEVENKVKHIVEQVTAGAPWD
LFKDAHDKLKLEKQKLIKERDSQKPDVNQSDVQKWVNKLVELADKARANDKEARLKCRTLINSICKSI
VIKLRGHDLKSEPVVTITFVTDEQFEFKVGKNGAVQFVDNGMAVNRRLKPLLA
CMp1 integrase
SEQ ID NO: 146
MLTVAYVRVSTDDQVEHSPDAQRRRCAEYAAQRNLGPVKFLSDEGQSGKDLERPAMQELVGLIEAGQV
TNLIVWRMDRLSRDSGDTSRLLRLFEQHCVNVHSLNEGDLETGSASGRFTAGLHGLLAQLEREKITEN
IHLGNEQAVRTGFWINRAPTGYDTVDRVLVPNDDAHLIRRAFKLRAGGQSYPQIEQGTGLKYSTVRHA
LENKVYLGFTRLRDERFPGKHEPLVSQAEFDAAQRAHTPGRRRGKDLLSGRVRCGECGRITGIDINGR
GKPIYRCRTRGNGCAIPGRSAAGLRRAARLSVELLRTDDQLVEAIRTHFAEKTERAGAGTAEPSRAGT
LGSLRSKRQKLFELLLAGKITDDFFAEQERQLTAKIEATEAHRTEAIETHRHHNALAEAFEQAAAMLR
DPAFELAEIWDNASAAERRVIVEELIESVTIYADRLEVNVTGAPPLLVTLAEVGLREPGTAPVVSEDL
TDFRSSGAWGLSGRTRLIDWVA
Pa19 integrase
SEQ ID NO: 147
MEINAVNHIKDVAIYLRKSRGQEEDLDKHREELVELCEKNGWRYVEYGEIGSSQKLMDRPELSRLLKD
IQEDMYDAVVVMDKDRLSREGVGQAQINKILAENDCLIVTPTRVYDLTNESDMMVSEFEDLMARFEYR
AIVKRLKRGKRRGAKQGKWTNGTPPIPYIYNPKFRDKTDKSIEKGEDLDSLIVDEEKLEIYRFIVDSF
LNGMSPYTIAWELNKRKIASPRGSTWNNTGIRRLLKDETHLGRIIANKTTGSKKRLSTGRLDYKVNKR
EDWIIVENCHKAVKTKEEHEKILIEMQKRKRDSYATTGENALSGLIVCSKCGATMAQKKSKIESERYS
YVEACKNFLEDGITKCDNHGGSTMYLMKEIERQLVMYRDDIEKENERVSKGGGLTKTIESEKKKKEER
IIELEDELEQVTTLALAKFFTPEEAVQQKKKILDNISKLESELYALNLQTDNLGNMTRSEKVNAINKF
LEVMDMPHISNSDLNRLYKTIIHSIIWDKSNPDELKVTINFL
Pg17 integrase
SEQ ID NO: 148
MKRAALLLRCSTNIQDYNRQKLELEKVANRFKLKVAKVFGEHVTGKDDIRKGNRKSVDELINACENNE
IDVVLISEVSRLTRNFLYGVTLVDKFNRDYSIPIYFRDKRKWTIDVETGEVNLEFEKELRKAFEQAEE
ELASIRFRMASGKRDSAGLGQWFAGFPPFGYTRQKNGYLVKNEYAPIVKEMEDKYLEEGQALITTTRY
IYGKYPDIKKLKSIGNTKFILNNKAYTGRAEANIYDEIDKVTDTFYFDIPAIIDEETYNKVQKKLANN
RTTTPYPKAAKYLLQKLIKCSICGAFYTRLHSNKRDTVHFKCTSDKNSLTGCKSQVYLNERIVNPIIW
NFVKEQLFYVGKMNAEQRLEAIAGENDNKSKAIEEQEALKISYKEQENKLTRLEDLYLDNDIDKNRYK
ERKSKIEKELSSIKAQMDRLTDRIRLSDENIKRENSTDFTEQYFKEVEADLEKQMKVLREYVKGIYPL
YIDKVYCFLKVDTIEGMFNIFYEPRKAQQKCAYFIKDTLAQYHPDIRNFYTPNNTLLTDSDEVDAYYT
LEEMKEICSRNGFEVRYRE
Sall integrase
SEQ ID NO: 149
MKVAIYTRVSTLEQKEKGHSIEEQERKLRAYSDINDWKIHKVYTDAGYSGAKKDRPALQEMLNEIDNF
DLVLVYKLDRLTRSVKDLLEILELFENKNVLFRSATEVYDTTSAMGRLFVTLVGAMAEWERTTIQERT
AMGRRASARKGLAKTVPPFYYDRVNDKFVPNEYKKVLRFAVEEAKKGTSLREITIKLNNSKYKAPLGK
NWHRSVIGNALTSPVARGHLVFGDIFVENTHEAIISEEEYEEIKLRISEKTNSTIVKHNAIFRSKLLC
PNCNQKLTLNTVKHTPKNKEVWYSKLYFCSNCKNTKNKNTCNIDEGEVLKQFYNYLKQFDLTSYKIEN
QPKEIEDVGIDIEKLRKERARCQTLFIEGMMDKDEAFPIISRIDKEIHEYEKRKDNDKGKTFNYEKIK
NFKYSLLNGWELMEDELKTEFIKMAIKNIHFEYVKGIKGKRQNSLKITGIEFY
E101 integrase
SEQ ID NO: 150
MPKTKTPKTAARVYRAASYARLSVDDESYGTSGSVLNQHAMIRDFADANGGLSIVAEYSDDGFSGSTF
ENRPGWGSLLADVETGKVDCIVVKDLSRLGRNYLDVSRYLDQVFPALGVRVVAITDGYDSAAEKTPAD
ALMLPVKNLFNDMYCRDASAKTKASLSAKRRRGEFVGAFAPYGYAKGSGPDRGRLVIDPEPAGVVRGI
FDARIGGMSAAGIAAMLNESLVPSPYEYFAAKGAARSSNFCKGERTAWDARTVLRILSNETYAGTLVQ
GKTCKPDFRRKAVLSVDESEWDRSPGAHEAIVDPATFGLVRKLARRDMRLSPGAKRSLPLSGFLFCAD
CGATMARHASRCSSGKRFYYSCETHRKNRAECGMHKVWEDELSAAVMRAVHGHALAAVDGDGVVRSAE
AYRHDRRAELACRLESVEGRIEHNGDLRLRMYSDYAAGVIDKAQYAELARAVDVRLQELKGEKAGIER
ETAQLDDVHAETWEQTLARYRDAEGLERVMVVELIDRVLVGEGGDIEVVERFGGPVAACATRQEGVA
Cp36 integrase
SEQ ID NO: 151
MKQLNIQSSSKITALYCRLSRDDELQGTSNSILNQKMMLEKYARDNNFTNLEFFIDDGYSGTNENRPD
WSRLQSLIDEGKIGCIIVKDMSRLGRDYLRVGYYTDIVFPEADIRFIAINNGIDSNESTENDLTPFIN
IINEFYAKDTSKKIRAVFKAKGESGKPLATIPPYGYLKDKEDKYKWVIDEEASKVVKKIFQLCVQGYG
PSQIASELIKEGIPTPTEHFDKLGINVSSPLSEIKGNWQPKTISLILEKMEYLGHTVNFKTYKKSYKS
KKKLENPKEKWQIFENTHEAIIDQETFDIVQRIRQGRRVRNNLGEMPALSGMLYCADCGAKLYQVRGK
GWEHEKEYFVCASYRKHKGLCTSHQIKNVQVEELLLHELKKITEYARQYEDDFVKLVQSKTQNELNKS
LKESKKDLVHVKERINKLDTIIQRLYEDMVEGKLSEDRFQKLSSNYETEQCELEKKATMLEKIIHDTE
QTTLNTTAFLKQVREHTTINKLTPEIIRMFVDKIIVEKPEKIEGTRTKKQTIWIYWNYIGILDIEKTA
Pc01 integrase
SEQ ID NO: 152
MRAAIVRRVSTLKEVQENSLQNQKDFFEDYVQSKGWDIAEIYTETETGTKFSRKEMNRLIADAKAKKF
DIILAKELSRLARNQRLALEIKEVIEKHGIHLVTLDGAIDTTTGNTHMFGLFAWIYEQEAQRTSERIK
MALETKAKKGLYKGSNPPYGYVVREGRLYVSDDGSSEIVERIFQSYLGGKGFDAIARELFEEGVPTPS
MIAGKKNQTIYWHGSSVRKILENPHYTGDMVQGRQSTISVTNKSRKEKPKTEFIVVKNTHESIISGEV
FESVQQLIAFNRKKSIDNPDVCSRPHQNVHLFTGVIFCPDCGSGFHYKRNGACYICGRSDKLGDKACS
KHRIREDALVAIIRWDLQRLSKLLDDQSFYNTVKDKFVKAKSKLEKELKACAGKIENIKNLKSKALSK
YLEESITKSDYDDYIAAQDAEIQKLLHNKEKLDSAISASVDVDVLGKIKGIVASSLEFQEINREVINR
FIEKIEVTADGNVKLYYRFAGTSKILNELMTDVN
Enc9 integrase
SEQ ID NO: 153
MTALYARLSQEDTLEGDSNSIVNQKAVLSKYAADNGFSNPVFFIDDGVSGVTFDRPNFNRMIAEIEAG
NVATVIVKDMSRLGRDYLKVGYYTEIFFVERDVRYIAINDGVDSAKGDNDFTPFCNLENDFYAKDTSK
KVRAIKRAQGQAGEHLTKPPYGYMVSPTDKKQWIVDEEAAAVVKQIFDLCIGGKGPMQIAKILKEDKV
LTVKAHYAEKKGKTFPDNPYNWNENSIVAILERMDYCGHTVNFKSYSKSHKLKKRIPTTKEQQAIFFN
THEAIVEDAVFERVQELRANKRRPTKADRQGMFSGLVYCADCGSKLHFATCKSENGSQDHYRCSNYKS
NTGSCTAHFIREEVLKQIIWSRIFDVTALFFDDIIAFKEMMYQQRSTETEKEMKRRKREVMQAQKRIV
ELDRIFKRIYEDDISGAISHDRFLKLSAEYEAEQRELEEKVKSEQQEVDTYEQNMSDFDSFSAIIRKY
VGIKELTPAIVNEFIKKIIVHAPEKADGKRVQKVDIVENFVGEINFLSATQPKRQGA
Cd16 integrase
SEQ ID NO: 154
MKQQIYNTALYLRLSRDDELQGESSSITTQKSMLRLYAKEHHLNVIDEYIDDGWSGTNFERPSFQRMI
EDIEAGKINCVVTKDLSRFGRNYIMTGQYTELYFPSHNVRYIAIDDGVDSEKGESEIAPFKNIINEWV
ARDTSRKVKSAFRTKFAEGAHYGAYAPLGYKKHPDIKGKLLIDDETKWIVEKIFSLAYQGYGSAKITK
ILREEKVPTASWLNFTRYGTFAHIFEGKPESKRYEWTIAHVKAILKSEVYIGNSVHNMQSTVSFKSKK
KVRKPESEWERVENTHEPIIDKEVFYRVQEQIKSRRRQTKEKATPIFAGLVKCADCGWSMRFGTNTAN
KTPYSYYACSFYGQFGKGYCSMHYIRYDVLYQAILERLQYWAKAVQQDEEKVLHKIQKAGNAERIREK
KKKASALKKAENRQNEIDRLFAKMYEDRACEKITERNFVMLSGKYQKEQIELEQQITSLREELSKMEQ
DMIGAEKWVELIKEYSVPKELTAPLLNAMIEKILIHEATTNEDNERIQEIEIYYRFIGKVD
Cd15 integrase
SEQ ID NO: 155
VVTKDLSRLGRNYIMTGQYTELYFPSHNVRYIAIDDGVDSEKGESEIAPFKNIINEWVARDTSRKVKS
AFKTKFAEGAYYGAYAPLGYKKHPDIKGKLLVDEETKWIVEKIFSLAYQGYGSAKITKILREEKVPTA
SWLNFTRYGTFAHIFEGKPESKRYEWTIAHVKAILKSEVYIGNSVHNRQSTVSFKSKKKVRKPESEWF
RVENTHEPIIDKEVFYRVQEQIKSRRRQTKEKATPIFAGLVKCADCGWSMRFATNKANKTPYSYYSCS
FYGQFGKGYCSMHYIRYDVLYQAVLERLQYWAKAVQQDEEKVLNKIQKVGNAERIREKKKKASALKKA
ENRQNEIDRLFAKMYEDRACEKITERNFIMLSGKYQKEQIELEQQITNLREELSKMEQDMIGAEKWIE
LIKEYSVPKELTAPLLNAMIEKILIHEATTNEENERIQEIEIYYRFIGKVD
Cd31 integrase
SEQ ID NO: 156
MPRIRKDKMARSYAEPFWKIGLYIRLSREDDNEDESESVINQEKILRDFVDSYFEPGTYVIVDVFADD
GLTGTDTARPNFKRLEGCIVRKEVNCMIIKSLARGERNLADQQKFLEEFIPINGARFICTGTPFIDTY
ANPHSASGLEVPIRGMFNEQFAATTSEEIRKTFKMKRERGEFIGAFAPYGYKKDPNDKNSLIVDEEAA
EVVKSIYHWFVNEGYSKMGIAKRLNQMGEPNPEAYKKKKGFKYNNPNSDKNDGLWSASTIARILQNEV
YTGVMVQGRNRVISYKVHKQINVPEEEWFVVPNTHEAIIDRETFEKAQALHKRDTRTAPGKQEVYLMS
GFVRCADCKKAMRRKTARDIAYYSCRSYTDKKICSKHSIRQDKLENAVLAALQMQIALVDRLAEEIER
INNAPVINRESKRLSYSLKQAEKQLKQYNDASDSLYLDWKSGEITKEEYRRLKGKIAEQIQQLEANIS
YLKEEMQVMADGIGTDDPYLTAFLKHKNIQSLNRGIMVELVKAVWVHENGEITVDENFADEYQRIIDY
IENNHNVIQVIENKAI
R109 integrase
SEQ ID NO: 157
MEKAVLYLRLSKEDIDKISEGDDSASIKNQRLLLTDYALKHDYQIVDVYSDDNESGLYDDRPDFERMI
QDAKLGKFSIIIAKTQARFSRNMEHIERYLHHDFPILGIRFIGVTDGVDTADSSNKKARQINGLVNEW
YCEDLSKNIRSAFKAKMKDGQYLGSSCPYGYIKDPTNHNHLIVDDYAADIVREIYKLYLQGIGKGRIG
RILSDRGVLIPSLYKRNVQGINYHNANAKAETHLWSYQTIHQILNNQMYLGNMVQYRTTTLSYKDKTK
KLRLPSEWIIVEGTHEPIISYAIFQRVQELQKIRTKEVNTEQKYTNIFSGLLYCADCNHTMNRNYTRK
GVFCGFICSTYKRHGNKAGCRSHRVDYDALCDAVLESIKLEARKILSDKDVDELKKSRLISSREEKIE
NEIRILENECEKLKQYKQKTYEAYLDDLITMNEYKVYIKKYDTELSDTCKKIDKLYAEKQVTDSLDQK
YKEWVNMFSDYINITELTRTVVIELIKRIDVYEDGNIKIHYRFKNPYESSK
cd08 integrase
SEQ ID NO: 158
MLQSNKITALYCRLSQEDMQAGESGSIQHQKMILQRYADEHHFLNTKFFVDDGFSGVSFEREGLQAML
QEVEAGRVATVITKDLSRLGRNYLKTGELIEIVFPENGVRYIAINDGVDTAREDNEFTPLRNWENEFY
ARDTSKKIRAVKQAQAQKGERVNGEYPYGYIPDPNNRHHLIPDPETAPIVKQVFAMFVSGVRMCEIQK
WLAENKVLTIGALRYQRTGQARYQRAMIAPYTWPDKTLYDILARQEYLGHTITAKTHKVSYKSKKTRK
NEEEQRYFFPNTHEPLVDEETFELAQKRIATRHRPTKAAEIDIFSGLLFCAGCGHKMYYQQGVNIEPR
RFSYSCGAWRNRARTGSECTSHYIRKNVLLDLVLEDMRRVLQYVKEHEQDFICKATEYGDMEARKALA
QQQKELSKAQARMTELDTLFRKLYEDNALGRLTDERFVFLTSGYEDEKKSLAARIDELQQQIATVTER
KRDISRFIQIVGKYSDIQELTYENVHEFIDRILIHELDRETNTRKIEIHYSFVGQVDTEQEPTQVVNH
DRRNMVDVKSIAI
attD sequence
SEQ ID NO: 159
ACTTAATATAAGGGAGATTACTTTTAAATTTTAATAGTAGTAGTGTTACAGGGTACGTAGTGCTTGTA
ACACTATTTTTATGTATAAAAAAAGACCACGCTCATAAGAAC0
attD sequence
SEQ ID NO: 160
TAGTGACGTCTGTCCGCGCAGTGATCGAGGGAGTGTGTGCTTTGCCGACTGGCAAGGTCAAGCCGGTC
TGCTAGGCACAGAGAGCCGGTACAGTCCTCCCCATGCAACCCAA
attD sequence
SEQ ID NO: 161
CGTAAAATCAGGCGATGCGCCGGCACATCGCAAATGTATTTTGACTCTCGTTCGGGTTGCCGAGCGTG
TCCGAAAATATATTATCAGACAACTCGGTCAGGGGAGCGCGTAAACGAAA
attD sequence
SEQ ID NO: 162
GGGTGTAGATGACTGCCTTCACCTGCATAGTTAAAACGGTAGCAGTGAAGCTACGATCTGTCATAGCG
TCCGAGCTCACTGTTTTAGGCTCTAACCGGTGCGGGACGTG
attD sequence
SEQ ID NO: 163
GCATTGCGTGGTGGGCTGGCCATATCCTAATTGTTGCACGGTTGCGGAATGTGGTGGAATTCCGCACC
GGATTAACAACTGCCAAAAAATAGAAACCCGCAGCTCACGGCATAT
attD sequence
SEQ ID NO: 164
CGAAATGGTTGGCGTTGAGGTCAATGATTAATGTGTATAGGGTTAACATTTAAATCAGTACAATCGTA
GACGCTCTACACTATTTTCTGTGTATAAAAATATCGAGAATAAACGCTT
attD sequence
SEQ ID NO: 165
ACGGCGCACGTCGTTCCGGTCTGGGAGCCTATGCACAACGAGGGATAACGGACGTTCTCCCATGCTGC
GCATAGGGCAGGCGTGCGTGCGGTTGCGCGTCGTAGG
attD sequence
SEQ ID NO: 166
CATATAGTTAATGTGTGGTTTGTTTTTTTGTACGGAAGTGTCTAAAAAGCGTCGCAATTTGCGGGGGT
TCCGTACATAATAATAGTCATTCGGGTACATCCATTTAGTGGA
attD sequence
SEQ ID NO: 167
TAATGAACATAGCACAACAAAACCAAAAAACAGATTTTACAAGGTTTTCCCGTTTAGTGTTAGCGAAA
TACTAAAACCTGATAAAAACCCTCTCCAGTTGTTTTTTCTTGCCCC
attD sequence
SEQ ID NO: 168
ATGGCGACATATAAGCGTTCGTGCTTTGTCGTCACCTTGTTGGTGTAATTAGGTTGACGCCAACAGGG
TGATAACACAAGAAGGACTTTTTATTTCTTCTATTATATATAGA
attD sequence
SEQ ID NO: 169
TGATTACGATCAGTGCCCTGGGAGGCGATTCCGGCATGGCTTATATCCAACACCACCGAGAGCGCTGT
TGTCGAGCGTGTAAGCCAGGACGAGGACGAGCACGCCCACGGGCACG
attD sequence
SEQ ID NO: 170
AAGAGTGTTCTAAAATAGAAGAAAATAAAAACATACACATAAAGACGCACGTAGATACGTGAGTTCCT
ACCCACTTGTTTTTTACTCTATCTTCTCTTGTTTCCAATTTCT
attD sequence
SEQ ID NO: 171
CACATTCCAAGATGTCTCAAAATCAGTCTCACAATCCCCGTATAGGTATAGTATCCCTCGGGTGCCCA
AAGTATATTGAAAATATACCAAGGTTGAATACCTCGTCAGCTAGGCTAG
attD sequence
SEQ ID NO: 172
CACCCGTCCTAGTACTCGCATATGGCGAGTCTACAACCGTTCCCATACGACAAGTCGTAGTACAACTA
TTGTAGATGGGTGTTTAGGGTACGAAAAAAGCCCCCAGGCCA
attD sequence
SEQ ID NO: 173
CCTGGAGAAGCCGATTCCATGTCATGTACGGTAGCTTGTTGTGTACAGGTTGATGTTCCACGAGCCGG
ACAGCAACCTCCACAAAACCTGAACAGCCCCCGGCGGTGGTGCGAACA
attD sequence
SEQ ID NO: 174
ATTCATTACAGGTAGAACTTGTTGACTAATAATTAGTGTAGTTTTACCTGTGCTGCACATCCAGACCA
GTTAAAACTCCATTAAAACACGTGATTATTTTCACACAAAAAAAACCTCTA
attD sequence
SEQ ID NO: 175
TCTTTTATGTCAGCAGTTCTTTTCATCCTGTTTCATCTTGTACGCTTCGTTTCGCCGAAGCGTACAAG
ATGGAAATCATAAACCTTCAAATGCGCCATTCGATCTTGATGGA
attD sequence
SEQ ID NO: 176
CTTGAAGATTATGTGAGGCGAACATCTAAAGAAAACCGAATAGACTTTATTCAAGGGTCATTGTATTG
TAGCTATTCGGCATTGTAGCATTAGCCATAACGGTTATAAGCTTA
attD sequence
SEQ ID NO: 177
GGTTCATTGAACAGGAGAACAATGATATGAGTGTTAAGGCAAGAATACTAGTGCTTTTACATAGCTAA
ACACGTACATTCAACTACCTGTTAATAACAGGACAATAT
attD sequence
SEQ ID NO: 178
TACTATATAAACTAAATAATAAATATTCTTGTTCACTTTGAAACTATATTGTGATATTGTTGCAAAGA
AGCAAAATTGATACTCTCTTATACTTTACTGGGGTG
attD sequence
SEQ ID NO: 179
ATCAATTGAAGAAGATGTAAGATGAACATCTAAAGAAAACCGAATAGACTTAAATCAAGGGTCATTGT
ATTGTAGCTATTCGGCATTGTAGCATTAGCCATAACGTTTATAAGTTCA
attD sequence
SEQ ID NO: 180
AACAGGTGATAGGATTCGGATGTTCTCATAGTGTATTTAGGGAATTAATATCAGTATAATGGGTCCCT
AAACACATCATTTTAGGTATTGAATATGAGACGGGC
attD sequence
SEQ ID NO: 181
AAAATATACTTGCGATTAGTCGTTATTTTTCATAGACAAGTAAATCAATCATGCACATGGTAGCATGA
GTGTTCTATGAAAAAAGAGGGTAGGAGCGATCAGCTA
attD sequence
SEQ ID NO: 182
TAACAGTAAATTTCCTTATATAATTTATGTGTACTAACTTATATATTCCGGAAGGATAATATAGGTTA
GTACACGTATAAAAATATCTTTTATATTGACACAATTTA
attD sequence
SEQ ID NO: 183
CCGTTGAAGAACTACGCTAAAAGTATTAAATCAAAATGTTCCCATAGTATACGAAAGGACGCATACCA
TAATCAATGGGAACATTTGAATTTTCGTAAAAAAAAGAGGCCA
attD sequence
SEQ ID NO: 184
AGAACCTTGAAAAACTATGGCTTATGCTACCTCGCCGAATAGCTCAAATACAATGACCCTTGAATATA
GGCTATTCGGGTTTTGAAGGTATCTGGTTTTTATTCACATCACAA
attD sequence
SEQ ID NO: 185
TAAGTGATTTCAGTCTGAGAGGGAAAGTGTATCAATAGAAAGGTCTCTTCAGGATTACACCTTTCTTA
TGATACACTTCTTCAATCCTTCATAACTCATCATAAA
attD sequence
SEQ ID NO: 186
TGAATTATTGATGTAAGAGGCTTTTTGTTCTTTAGTGTTCTTAACTTCCTTAATGTCTGCTATTGTAG
TGCTATCTACACTACGGTTAAGCGACCTCTCTAATGAAAA
attD sequence
SEQ ID NO: 187
GTAACGCTCTTCGAGAAAGCAGATTCTCATATCCATCTTGAGTCTTCTTTCTCGCAAGACAACACGAA
ATAGACACAGTCTCTTCCCTAGCTGTACACTGAGCC0
attD sequence
SEQ ID NO: 188
GTTCAACCGTCCTAGAAGACCTTGATGTGTGAGATTCACCCCTACCATTCGAGACTGGCAGGTGGTAT
TCTCACACTTCCTAAGATCTCAGCAGGAAGCCCG5
attD sequence
SEQ ID NO: 189
CACCACCTATTAATTTAGGAGTGTGGTTGTTTTTGTTGGAAGTGTGTATCAGGTAACAGCATAGTTAT
TCCGAACTTCCAATTAATAAAACTCTATACCCGTAATCTTC0
attD sequence
SEQ ID NO: 190
CCTATTAATTTATGAGTGTGGTTGGTTTTTAATGAATGTTTTGTAACTATTGCGTTCTTTCTAGTTAC
ATAACACTCATTAATATTTGAAATGTATTTCATTGATT
attD sequence
SEQ ID NO: 191
TGACAGCGCCATTTGCTGGGAGAGAGTGATGGTGTAACAAGCGAATTACTTGGGATCAGATCATCTGA
ATGTTACACTGCCACCTTTTCGACGAAGGTGTGTGGTTTC
attD sequence
SEQ ID NO: 192
AATTGCCATGGAAAAACTAAACCTGTCGGCAAGAGCCTATGATCGAATTTTAAAAGTATCAAGAACTA
TTGCCGATTTAGCATCCGAAGAAAATATAAAATCGGAACA
attD sequence
SEQ ID NO: 193
GTACAAATAAAAGCATCAAGACACCGATAATTAACAGGACAATCAACATCTCCACAAGTGTGAAAGCT
TCAACAGATTTTTTACGTAATTTTTTCCATAGTT
attD sequence
SEQ ID NO: 194
AAAAAGGAGAAGTTTATCTTCAAGATCCAATAGGGGTTGATAAAGAAGGAAATGAAATTTGTTTAATA
GATGTTTTAAGTAGTGAAAAAGATTTAGTTTTAGAAAA
attD sequence
SEQ ID NO: 195
TAATAAAGAATGAAAATAAATCTACTATTTTAGTTACCCATGATATATCAGAAGCTATTTCTATGTCA
GATAAGGTCGCAGTTTTATCCAAACGTCCTGCATCT
attD sequence
SEQ ID NO: 196
AATTTGAGTTTTAATTTATTTTTTTCTTTCGCAATTCTAAATTTTTGTAACATTTGTAGTTCCTCCTT
CATTCGAAATCATCGATAGTTAATTCTGAAACTCTCTTTTCATAGATATATAAATAATAGT
attD sequence
SEQ ID NO: 197
CGGAGGGTGATTTTAAAGAGTTTTTCATAATTAATACTTTGCACTCTGTTATTTTTTTTATAATTTAC
TATTTTTTTCAAATAATAACTATAAATATCTTTGCGTAGACAACCTTTAAATTTCGCTCAGAACC
attD sequence
SEQ ID NO: 198
TAAAGGGTTTCACGCATAAGTACCAATAATGTAACAACCTGTACTGAATGTGCTTCCAGTACAGGTTG
TTACACCGTTAGGCAAAAAAATAAATATCCATAG
attD sequence
SEQ ID NO: 199
TGATTGTCGCAGGACTATCCGACTGGGGGTGTGTTTTGATACCAACCGGGCTCCCGAACGGTATCGAA
ACACACCCCCACACGAAAGTGCGGGGACAGGCTTAAT
attD sequence
SEQ ID NO: 200
AGTTGATCCAGGTGGAAAAGGCGATCGAAGTTCATTGAGGGGTTCTATTTTTGAACGAGTTAACTGAG
TTTAACCCATACCATTCTCAGGGATTAAGAGGATATT
attD sequence
SEQ ID NO: 201
GAAAAAAATGATGACATTATTGAAAAAAGCGAAGGTTAAAGCTTTCACACTTATTGAGATGTTGGTTG
TCTTGCTCATTATCAGTGTGCTTCTCTTGCTCTT
attD sequence
SEQ ID NO: 202
AAATGCTGGACGGGAGGATAACAGAGGCTCAGTGTTACATACGATATTGTTGGGAGCAAGCTTTTCGT
AAGTAACACTGAGATATAGACGCCACTGGCTGTGGCT
attD sequence
SEQ ID NO: 203
CAAAATATGTGATGGAAGATATGAGGTTTTTACAACAGGAGAATTTCGTTTGTCTTTATCTCAATTCA
AAAAATCAAGTTCTTCATAAACATACTGTGTTTA
attD sequence
SEQ ID NO: 204
TCGATCAGGTCGACCAGCGTGCTGGCTTCGAGTTTGTGCATGAGGCGTTTCTTGTAAGTGCTGACCGT
CTTGCTGCTTAGAAACATGCCCTTTGCAATCTCCTTGT
attD sequence
SEQ ID NO: 205
CAATGAGATATGCGTTAGTTAGAACAATAGGTTGTCGGGCGCTTCTGCCAATTGAAAATCCGCGTGTC
GGTGGTTCAAATCCGCCTCCGGGCACCATTAACTTGCTGAAAATGCTAAGTTTTTGGAAAGCTCTTTG
ATCCCGCTTGTG
attD sequence
SEQ ID NO: 206
GCGTCATGCTCATCGAACAAATCTACAGAGCCTTTAAGATCATGAAAGGCGAAGCATATCACAAATAA
AACTAAAAAATAGATTGTGTATAATATATTTTAAATATAAAAAGGATTGATTTTATGTTA
attD sequence
SEQ ID NO: 207
CGAAATACATGATGGAAGAAATGCGTTTTTTACAACAAGAGCATTTTGTTTGTTTATATTTAAATACA
AAAAATCAAGTTATACATAGGCAAACGATCTTTAT
attD sequence
SEQ ID NO: 208
GTAATCCTCCCGTCAGACCGTTACCCTGTGTAGTCCCTTGTAAACTGTACTTTAGGTCAGTTTACAAG
GAACTACACGCAGACCGTGAAACGGGCTGCTGACAAC
attD sequence
SEQ ID NO: 209
CAGTTCCAATGGTTCTCAGAAAATTTCAAGTTAAAGCATTTACTGTTTTGGAGAGCCTTATTGTATTA
TCAGTAGTGGCATTTATGACGTTAGTATTTTCAA
attD sequence
SEQ ID NO: 210
ATCGCACGCTCTTCGTGGCAGGGAAAGCCGCAGTGTAACATTCAGAGAAACTGGTCACAACATTTTCT
TTTGTTACACCACGTTGATGGCTAGCCACCTATGCACC
attD sequence
SEQ ID NO: 211
AGTTTAATACTAAAAGAGGTATATTAATTTTATTTAAAAATTGACATTAATGACTCTTAACCAAATGC
TCTGCTTGTGAAAAAAGCTTTATAAAACTTTATAAATAGGTT
attD sequence
SEQ ID NO: 212
GCTGCTCAAAAGGGCAGCATACATCACAGTGTCACTTAGCACATAGCTGTTCACAGGTATTTTATATG
TTACACCACCAGCCCATTCTGCTGGCAATACTAG
attD sequence
SEQ ID NO: 213
ATTTGCTGGCTGAGATGGTAACAGGGGATCAGTGTTACATGTGAAAACGTTGGGAGCAAGCTCTTTGT
AAGTAACACTGAGAAGTACCTAACATGAGAGTAACCC
attD sequence
SEQ ID NO: 214
TACTCCCTGGTGTCCTCCCAGCAAGCGCACTACTGGGTTAGGATGTTCACTGACACCAGTACAGTATC
AGAAGCATAAGTGGCAGGACGAATACCCAACTGAAATCAGTA
attD sequence
SEQ ID NO: 215
TGATTTTACGCTGGTGCTATATCCTAAACTCCCACAGATAAACAGTTAATGGTAATGAAATAACATTA
ACTGTTTATCTGTGTTTAATGCCTTAACTTAATCTAGTAGGAGGG
attD sequence
SEQ ID NO: 216
TTAAAAAGAAAGGCTTTAAGTTTGTGGGCGAGACGATTTGCTACGCCTTCATGCAAGCAGTAGGCATG
GTCGATGACCACATTGTTGGCTGTCCTAAAAAGC
attD sequence
SEQ ID NO: 217
GACACACTTTCGAGTGTGTCTTTTTTATTACCTGAATAAAACCAAGAACTAAAGTACCTAGTTTTATT
CAGGTAAGTGTAAAAAACGGCTAAATCTAGCCGTT
attD sequence
SEQ ID NO: 218
TTGGAATGACAATCAACAATAAGACAGAAATAACCATTAATACGATTAGCATTTCGATTAACGTAAAC
CCCTTTTCATTCTTCATTGTCCTTCCCTCCTATAAAT
attD sequence
SEQ ID NO: 219
CGTACATGGCTCCTAGTGTGTACGATGGGAAATAACCAAAAGAGCCGTCTGTCCAATGGATGTCTTGC
ATACAACCATTTTTGAAGTTGCCCTCTGTAGATAG
attD sequence
SEQ ID NO: 220
TTTGCGGGACGCTTCCGACGCTGTAGGACCGAGTGGACGGCCTGAAGCTCACTTCTAATCCGACGGTT
GCAGGTTCGAGTCCTGCCGGGGGCACTTCTAAAACCCTTGCAAGCAAAGGGTTCCTAGCGCCAACAGC
TGAGCTGTGCA
attD sequence
SEQ ID NO: 221
TAGATATTTCTCTTTATTTAAATAGTTGGTCGTAAATTACCACATGCTATTGGGGAGAAGTAGTGTAA
AAGGTACTTGGTCTGTGTTATTCGCCATATATCAAAAA
attD sequence
SEQ ID NO: 222
ATCTGTCCGCCCAATCGGCGGCAGAATGTAAGCTGACGGAATTCGGCTTGATCAATATGGATGAATTT
GATAAATTTACTCCTCGCAAGATGGCATTGTTGAAGAA
attD sequence
SEQ ID NO: 223
CTAAGATCAATACGATGTATCTTGTTATTACTTTTGCATCCATTTGTTTGCTCCTTTTATCCAAAATA
AAAAACGACTAAATAAGCCGTCTATTTGATATTTATATTATGGTGTGTTAATTTATATATAGA
attD sequence
SEQ ID NO: 224
CCATGGATTTCTCAGAGAACTCCGGGCGCGTTTTTAATATGCGCAAAGGGATCCCTTTTGTCAAGACG
AAAAAACAAGATTACCTGCAGAAATGCTCCGACTA
attD sequence
SEQ ID NO: 225
CTTAACCGCTTTTGAATGTCCGTCTTAGTTAATCCCTAATTGAACGCTCCAAAAGGTGACTTCCAATA
GGGATTTATTCCTTTTAAAATTAACGGCATAATCGT
attD sequence
SEQ ID NO: 226
CGTTTATCGGGCGGTAATATTTTAAAGTATTGCGCTACACTCGGCACCCGACACATGTGGAGTGCTGT
GTGTCGCTCGTATGGAAGTAATGATTAGGAGCCGCATTTACCTTTC
attD sequence
SEQ ID NO: 227
TGTCAGCGTTAATGATAAGTTGGTTTCTATACTTCCTTATCACTGTAAACATCAAGGTTTGCGGTGAT
AAGGAAGTAATTTCAGATTAGGCGGTATAGCCCCAT
attD sequence
SEQ ID NO: 228
TTAAAAAAGTCTGCTAATAAAGGCAATAATTCTATATCTGGGTAAGAATTTCCACTCTCCCACTTAGA
TACTGCTGGTGTTGACACACCTATAAACTTTGCTAATT
attD sequence
SEQ ID NO: 229
TCTTTGTTAAATTACGCAAAATTTCATTCCCAACTTCATATCCAAATAAATCATTTATATATTTAAAT
TTATTGACATCCAATTGTACTAAGTAAAAATTATCTTGATAATAATTCTTTAAA
attD sequence
SEQ ID NO: 230
AAGCCGCATGGTTACGGCATTTTCCGTGTTGTGCATATATTGACGGAGCAGTAGAGCCGTGTATTTAT
GCACAACATTAGATTTTCCTTTGTTTTGAGTAGG
attD sequence
SEQ ID NO: 231
AGAAACTTTACTCAAATGTATTTCTGTTGCCAGTGCAAATGATGCCTGTGTTGCTATGGCTGAATATA
TTTCAGGAAATGAAGAGGAATTTGTCCGTCAGAT
attD sequence
SEQ ID NO: 232
TGATAATAAAATATAACAGTTATTAAATCCCTTGGAATATATGAAATCTCAATTCCAGTTTGCCGAAA
TATCTGGCTAACTTTCCCAATTTTACAGACATTCCAGCCCATTC
LSR amino acid motif
SEQ ID NO: 233
[AEILSTVY]-[ADEGKQRST]-x (3)-[EG]-x-[ACFLMV]-x-[AFILMTV]-x (2)-
[FHILMNV]-[AGSV]-[ADILSTV]-x-[AGS]-x (3)-[KRSV]-[ADEGKNST]-
[AEIKMNQST]-[FILMST]-x-[DELQSV]-[ENQR]-x (4)-[AFHIKLMNQRSV]-x-
[AEGHKLMNQRSV]
LSR amino acid motif
SEQ ID NO: 234
[AGI]-[DEGNPSTV]-[DGNQS]-[AHNQRTVY]-x-[ADEHILPQRTY]-[ADEQR]-
[FIKL]-x-[DEFGNQRSTV]-[AILSTV]-[DEIKLNQRSTV]-[ADEKMNRSTV]-
[AGQRST]-x-[ADEKLQRT]-x-[ALMV]
LSR amino acid motif
SEQ ID NO: 235
[ADFILMNSY]-x (2)-[AIKMSV]-x-[AFGILMV]-x (3)-[QRT]-[AGS]-x-
[DEGNQS]-E-S-x-[AHKNRSTV]-K-x (2)-[LMRY]-[AINQSTV]-
[AEFIKLNRTV]-x-[AFHLNQSTY]-[AILMNRSTVY]
LSR amino acid motif
SEQ ID NO: 236
[EKNTGSLDVARP]-[EHITGSLDVAP]-x-[MITSLVARP]-[EKNITGSDQVARP]-
[EGSDARP]-[ILDAR]-[MHKTLVQDAR]-[EKITGSLDQVA]-[EKHDQVAR]-
[MHI SLVQAR]-[QEKNMSLDVAR]-[EKHGSLDQAR]-[EYKNIHLVA]-X-
[EKITGSLDQAR]-[EKHTGDQAR]-x-[QEKNTGSDVAR]-[QEKNTGSVDAR]-
[ISWLVFAR]-[QEMTGSLVDA]-[EKNITGSDARP]-[EMILDQA]-[EYILVFAR]-
[EMTGSLDVAR]-[EKNGSLDQAR]-[QEGVDARP]
LSR amino acid motif
SEQ ID NO: 237
[ADEHKNQRS]-[ADEFGHKMNQRSWY]-[EFY]-[FHLWY]-x-[ADEFIKLMNQRSTY]-
[FIQSTV]-[AGKLNRSTV]-[ADEHKNQRTY]-[INQR]-[FILMQS]-x (2)-
[AGKNS]-[KMQRSTV]-x (2)-[AEGKMNSTY]
LSR amino acid motif
SEQ ID NO: 238
W-[AEHNRSTV]-x-[AGNST]-[FGLMNQSTV]-[ILPV]-x (2)-[ILTV]-x (4)-
[ACGMQRST]-x-[ILVY]-G-[DEHNQS]-x-[EHILMQRT]-[AEFHLNPY]-
[CFHKMNQRTY]-[DEFIKLNQRSTV]
LSR amino acid motif
SEQ ID NO: 239
[AGINSTV]-x-[AIS]-x-[FILMY]-E-[IR]-x (2)-[DILT]-x-[AEIKMQS]-R-
[ITV]-x-[ADGRST]-x-[FKLMY]-[AEHIKLMNQRVWY]-x-[AIKLMR]
LSR amino acid motif
SEQ ID NO: 240
[FY]-[DEKQS]-[EKLMQ]-[KLR]-[KLV]-x-[GN]-[DEHKLMR]-[ST]-x-
[FHIQSTVW]
LSR amino acid motif
SEQ ID NO: 241
[ILV]-x (2)-[ADFHILMNQSVY]-x (3)-[AGS]-x-[DEIKNQRS]-[EQ]-S-x (2)-
[AK]-[AQRS]-x-[LMR]-[ILQRSV]-x-[ADEGHIQRS]-[AKNQSTV]-[AHKRWY]-
x-[AGHIKQRST]-x-[CHIKLRV]
LSR amino acid motif
SEQ ID NO: 242
R-[LMQR]-[ANS]-[NPST]-W
LSR amino acid motif
SEQ ID NO: 243
[ILV]-[AV]-x-[AFHILQWY]-[IMV]-x-[ELQT]-[AIV]-F
LSR amino acid motif
SEQ ID NO: 244
R-[DKNRSV]-[ADEFGKPQS]-[AEIKLSTV]-x-[FGILNV]-[AFILQRVY]-
[DEILMNQSTV]-[DEFILMQTVY]-[IKLRV]-[DEKNQR]-[DEFKLNQWY]-[FL]
LSR amino acid motif
SEQ ID NO: 245
[AEFILMNQSTVY]-[AFGILMRSTV]-x (3)-[ADEFGHLMNST]-x (2)-[DMNS]-
[DEQ]-x-[CFHLTVY]-x-[AEKLRY]-x (2)-[ALS]-x-[DEKNQRS]-[GIMQRTV]-
[DHKNQR]-x-[AGILNSTV]-[FHI KLMNQVWY]

### Example 3: Transgenic Animals

A system for stably integrating one or more nucleic acid sequences into a genome of a cell as provided herein is delivered to an embryonic stem cell of a non-human mammal (e.g., a mouse) to integrate a donor nucleic molecule containing a desired transgene into the genome of the embryonic stem cell.

In some cases, (a) a genome-editing system comprising (i) a polypeptide comprising a DNA binding domain and, optionally, a polymerase and (ii) a nucleic acid comprising a guide sequence that is complementary to a target site within said genome and a sequence that encodes an attA sequence; (b) a donor nucleic acid molecule comprising a transgene and an attD sequence; and (c) an integrase that targets said attA sequence and said attD site and can facilitate recombination between said attA site and said attD site are delivered to an embryonic stem cell of a non-human mammal (e.g., a mouse) to integrate the donor nucleic molecule containing the desired transgene into the genome of the embryonic stem cell.

The embryonic stem cell containing the transgene is injected into an inner cell mass of a blastocyst, and the blastocyst is then implanted into the uterus of female non-human mammal (e.g., a female mouse). Transgenic mice are selected from the offspring.

### Example 4: Knock-out Animals

A system for stably integrating one or more nucleic acid sequences into a genome of a cell as provided herein is delivered to a non-human animal model (e.g., an adult mouse having a particular disease) to integrate a donor nucleic molecule containing a knock-out cassette into the genome of one or more cells within the non-human animal model.

In some cases, (a) a genome-editing system comprising (i) a polypeptide comprising a DNA binding domain and, optionally, a polymerase and (ii) a nucleic acid comprising a guide sequence that is complementary to a target site within said genome and a sequence that encodes an attA sequence; (b) a donor nucleic acid molecule comprising a knock-out cassette and an attD sequence; and (c) an integrase that targets said attA sequence and said attD site and can facilitate recombination between said attA site and said attD site are delivered to a non-human mammal (e.g., a mouse) to integrate the donor nucleic molecule containing the knock-out cassette into one or more cells within the non-human animal model.

### Example 5: Generating Engineered T Cells

A system for stably integrating one or more nucleic acid sequences into a genome of a cell as provided herein is delivered to T cells to generate engineered T cells such as CAR T cells.

In some cases, (a) a genome-editing system comprising (i) a polypeptide comprising a DNA binding domain and, optionally, a polymerase and (ii) a nucleic acid comprising a guide sequence that is complementary to a target site within said genome and a sequence that encodes an attA sequence; (b) a donor nucleic acid molecule comprising a transgene encoding a particular receptor (e.g., a TCR or a CAR) and an attD sequence; and (c) an integrase that targets said attA sequence and said attD site and can facilitate recombination between said attA site and said attD site are delivered to T cells (e.g., T cells obtained from the mammal to be treated) to integrate the donor nucleic molecule containing the transgene encoding the particular receptor (e.g., the TCR or the CAR) into the T cells such that the particular receptor is expressed by the T cell (e.g., to generate an engineered T cell).

### Example 6: Treating Cancer

A system for stably integrating one or more nucleic acid sequences into a genome of a cell as provided herein is delivered to T cells (e.g., T cells obtained from a mammal (e.g., a human) having cancer).

In some cases, (a) a genome-editing system comprising (i) a polypeptide comprising a DNA binding domain and, optionally, a polymerase and (ii) a nucleic acid comprising a guide sequence that is complementary to a target site within said genome and a sequence that encodes an attA sequence; (b) a donor nucleic acid molecule comprising a transgene encoding a receptor (e.g., a TCR or a CAR that can target an antigen expressed by cancer cells within a mammal) and an attD sequence; and (c) an integrase that targets said attA sequence and said attD site and can facilitate recombination between said attA site and said attD site are delivered to T cells (e.g., T cells obtained from the mammal to be treated) to integrate the donor nucleic molecule containing the transgene encoding the particular receptor (e.g., the TCR or the CAR) into the T cells such that the particular receptor is expressed by the T cell (e.g., to generate an engineered T cells).

The generated engineered T cells are administered to the mammal (e.g., a human) having cancer to treat the mammal.

### Example 7: Treating Diseases Associated with Nucleotide Repeats

A system for stably integrating one or more nucleic acid sequences into a genome of a cell as provided herein is delivered to a mammal (e.g., a human) having a disease associated with nucleotide repeats (e.g., C9orf72 amyotrophic lateral sclerosis and frontotemporal dementia (C9 ALS/FTD)) to integrate a donor nucleic molecule containing a nucleic acid encoding a therapeutic gene product (e.g., a wild type C9orf72 polypeptide) to treat the mammal.

In some cases, (a) a genome-editing system comprising (i) a polypeptide comprising a DNA binding domain and, optionally, a polymerase and (ii) a nucleic acid comprising a guide sequence that is complementary to a target site upstream of a G4C2 repeat within said genome and a sequence that encodes an attA sequence; (b) a donor nucleic acid molecule comprising a splice acceptor, at least a portion of a wild type C9orf72 gene, and transcription termination signal and an attD sequence; and (c) an integrase that targets said attA sequence and said attD site and can facilitate recombination between said attA site and said attD site are delivered to cells within the mammal to integrate the donor nucleic molecule containing the splice acceptor, the at least a portion of a wild type C9orf72 gene, and the transcription termination signal into the cells such that a wild type C9orf72 polypeptide (e.g., a C9orf72 polypeptide lacking G4C2 hexanucleotide repeats associated with the C9 ALS/FTD) is expressed by the cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for stably integrating one or more nucleic acid sequences into a genome of a cell, the system comprising:
(a) a genome-editing system that can insert an acceptor attachment site (attA) sequence into a target site within said genome;
(b) a donor nucleic acid molecule comprising a nucleic acid cargo and a donor attachment site (attD) sequence; and
(c) an integrase that targets said attA sequence and said attD site and can facilitate recombination between said attA site and said attD site.

2. The system of claim 1, wherein said cell is a mammalian cell.

3. The system of claim 2, wherein said mammalian cells is a human cell.

4. The system of claim 1, wherein said cell is a plant cell.

5. The system of claim 1, wherein said cell is a prokaryotic cell.

6. The system of any one of claims 1-5, wherein said genome-editing system comprises (i) a polypeptide comprising a DNA binding domain and (ii) a nucleic acid comprising a guide sequence that is complementary to said target site within said genome and a sequence that encodes said attA sequence.

7. The system of claim 6, wherein said DNA binding domain is present in polypeptide selected from a Cas9 polypeptide,a Cas12 polypeptide, a zinc finger polypeptide, and a transcription activator-like effector (TALE) polypeptide.

8. The system of claim 6, wherein said polypeptide comprising said DNA binding domain comprises a polymerase.

9. The system of claim 8, wherein said polymerase is a reverse transcriptase (RT) selected from the group consisting of a Moloney murine leukemia virus (M-MLV) RT, an avian myeloblastosis virus (AMV) RT, and a human immunodeficiency virus type 1 (HIV-1) RT.

10. The system of any one of claims 1-9, wherein attA sequence comprises from about 20 to about 100 nucleic acids.

11. The system of claim 10, wherein said attA sequence comprises any one of SEQ ID NOs:11-84 and SEQ ID NO:254.

12. The system of any one of claims 1-9, wherein attD sequence comprises from about 20 to about 100 nucleic acids.

13. The system of claim 12, wherein said attD sequence comprises any one of SEQ ID NOs: 159-232.

14. The system of any one of claims 1-13, wherein said integrase is a large serine recombinase (LSR).

15. The system of claim 14, wherein said LSR comprises an amino acid sequence containing a motif set forth in any one of SEQ ID NOs:233-245.

16. The system of claim 14, wherein said LSR comprises or consists of an amino acid sequence having at least 70% sequence identity to the sequence of any one of SEQ ID NOs:85-158.

17. The system of claim 14, wherein said LSR comprises or consists of an amino acid sequence set forth in any one of SEQ ID NOs:85-158.

18. The system of any one of claims 1-17, wherein said donor nucleic acid molecule is from about 250 nt to about 30 kb.

19. A method for stably integrating one or more nucleic acid sequences into a genome of a cell, the method comprising administering to said cell:
(a) a genome-editing system that can insert an attA sequence into a target site within said genome;
(b) a donor nucleic acid molecule comprising a nucleic acid cargo and an attD sequence; and
(c) an integrase that targets said attA sequence and said attD site;
wherein said genome-editing system integrates said attA sequence into said target site, and
wherein said integrase facilitates recombination between said attA sequence and said attD sequence thereby integrating said donor nucleic acid molecule into said genome of said cell.

20. The method of claim 19, wherein said cell is selected from the group consisting of a T cell, a natural killer (NK) cell, a non-human embryonic stem cell, an induced pluripotent stem cell (iPSC), a hematopoietic stem cell (HSC), a liver cell, a muscle cell, a monocytes, a B cell, a neuron, an astrocyte, and a microglial cell.

21. The method of claim 20, wherein said cell is a T cell and wherein said nucleic acid sequence encodes a chimeric antigen receptor polypeptide or an engineered T cell receptor.

22. The method of claim 20, wherein said cell is a NK cell and wherein said nucleic acid sequence encodes a T cell receptor or an engineered natural killer cell receptor.

23. The method of any one of claims 19-22, wherein said cell is a mammalian cell.

24. The method of claim 23, wherein said mammalian cells is a human cell.

25. The method of any one of claims 19-22, wherein said cell is a plant cell.

26. The method of any one of claims 19-25, wherein said genome-editing system comprises (i) a polypeptide comprising a DNA binding domain and (ii) a nucleic acid comprising a guide sequence that is complementary to said target site within said genome and a sequence that encodes said attA sequence.

27. The method of claim 26, wherein said DNA binding domain is present in a polypeptide selected from a Cas9 polypeptide, a Cas12 polypeptide, a zinc finger polypeptide, and a TALE polypeptide.

28. The method of claim 26, wherein said polypeptide comprising said DNA binding domain comprises a polymerase.

29. The method of claim 28, wherein said polymerase is an RT selected from the group consisting of a M-MLV RT, an AMV RT, and a HIV-1 RT.

30. The method of any one of claims 19-29, wherein said attA sequence comprises any one of SEQ ID NOs:11-84 and SEQ ID NO:254.

31. The method of any one of claims 19-29, wherein said attD sequence comprises any one of SEQ ID NOs: 159-232.

32. The method of any one of claims 19-29, wherein said integrase is a LSR.

33. The method of claim 32, wherein said LSR comprises an amino acid sequence containing a motif set forth in any one of SEQ ID NOs:233-245.

34. The method of claim 32, wherein said LSR comprises or consists of an amino acid sequence having at least 70% sequence identity to the sequence of any one of SEQ ID NOs:85-158.

35. A method for labelling a polypeptide encoded by an endogenous nucleic acid within a cell, the method comprising administering to said cell:
(a) a genome-editing system that can insert an attA sequence into a target site within said genome;
(b) a donor nucleic acid molecule comprising a nucleic acid cargo encoding a detectable label and an attD sequence; and
(c) an integrase that targets said attA sequence and said attD site;
wherein said genome-editing system integrates said attA sequence into said target site, and
wherein said integrase facilitates recombination between said attA sequence and said attD sequence thereby integrating said donor nucleic acid molecule into said genome of said cell such that said cell expresses a fusion polypeptide comprising said polypeptide encoded by said endogenous nucleic acid fused to said detectable label.

36. The method of claim 35, wherein said detectable label is selected from the group consisting of a HiBiT tag, a HaloTag, a Flag tag, a HA tag, a MS2/PP7 tag, a Sun/Moon tag, a poly(His) tag, a mCherry polypeptide, a green fluorescent polypeptide (GFP), a glutathione-S-transferase (GST), a luciferase, a horseradish peroxidase (HRP), an alkaline phosphatase (AP), and a apurinic/apyrimidinic endodeoxyribonuclease 2 (APEX2) polypeptide.

37. The method of any one of claims 35-36, wherein said cell is a mammalian cell.

38. The method of claim 37, wherein said mammalian cell is a human cell.

39. The method of any one of claims 35-36, wherein said cell is a plant cell.

40. The method of any one of claims 35-39, wherein said genome-editing system comprises (i) a polypeptide comprising a DNA binding domain and (ii) a nucleic acid comprising a guide sequence that is complementary to said target site within said genome and a sequence that encodes said attA sequence.

41. The method of claim 40, wherein said DNA binding domain is present in a polypeptide selected from a Cas9 polypeptide, a Cas12 polypeptide, a zinc finger polypeptide, and a TALE polypeptide.

42. The method of claim 40, wherein said polypeptide comprising said DNA binding domain comprises a polymerase.

43. The method of claim 42, wherein the polymerase is a RT selected from the group consisting of a M-MLV RT, an AMV RT, and a HIV-1 RT.

44. The method of any one of claims 35-40, wherein said attA sequence comprises any one of SEQ ID NOs:11-84 and SEQ ID NO:254.

45. The method of any one of claims 35-40, wherein said attD sequence comprises any one of SEQ ID NOs: 159-232.

46. The method of any one of claims 33-38, wherein said integrase is a LSR.

47. The method of claim 46, wherein said LSR comprises an amino acid sequence containing a motif set forth in any one of SEQ ID NOs:233-245.

48. The method of claim 46, wherein said LSR comprises or consists of an amino acid sequence having at least 70% sequence identity to the sequence of any one of SEQ ID NOs:85-158.

49. A method for making a non-human transgenic organism, the method comprising administering to an embryonic stem cell of said organism:
(a) a genome-editing system that can insert an attA sequence into a target site within said genome;
(b) a donor nucleic acid molecule comprising a transgene and an attD sequence; and
(c) an integrase that targets said attA sequence and said attD site;
wherein said genome-editing system integrates said attA sequence into said target site, and
wherein said integrase facilitates recombination between said attA sequence and said attD sequence thereby integrating said donor nucleic acid molecule into said genome of said cell such that said cell expresses said transgene.

50. The method of claim 49, wherein said cell is a non-human mammalian cell.

51. The method of claim 49, wherein said cell is a plant cell.

52. The method of claim 51, wherein said transgene expressed by said plant cell comprises a herbicide resistance polypeptide.

53. The method of any one of claims 49-52, wherein said genome-editing system comprises (i) a polypeptide comprising a DNA binding domain and (ii) a nucleic acid comprising a guide sequence that is complementary to said target site within said genome and a sequence that encodes said attA sequence.

54. The method of claim 53, wherein said DNA binding domain is present in a polypeptide selected from a Cas9 polypeptide, a Cas12 polypeptide, a zinc finger polypeptide, and a TALE polypeptide.

55. The method of claim 53, wherein said polypeptide comprising said DNA binding domain comprises a polymerase.

56. The method of claim 55, wherein the polymerase is an RT is selected from the group consisting of a M-MLV RT, an AMV RT, and a HIV-1 RT.

57. The method of any one of claims 49-56, wherein said attA sequence comprises any one of SEQ ID NOs:11-84 and SEQ ID NO:254.

58. The method of any one of claims 49-56, wherein said attD sequence comprises any one of SEQ ID NOs: 159-232.

59. The method of any one of claims 49-56, wherein said integrase is a LSR.

60. The method of claim 59, wherein said LSR comprises an amino acid sequence containing a motif set forth in any one of SEQ ID NOs:233-245.

61. The method of claim 59, wherein said LSR comprises or consists of an amino acid sequence having at least 70% sequence identity to the sequence of any one of SEQ ID NOs:85-158.

62. A method for making a non-human organism having reduced or eliminated levels of a polypeptide, the method comprising administering to an embryonic cell of said organism:
(a) a genome-editing system that can insert an attA sequence into a target site within said genome;
(b) a donor nucleic acid molecule comprising a nucleic acid cargo and an attD sequence; and
(c) an integrase that targets said attA sequence and said attD site;
wherein said genome-editing system integrates said attA sequence into said target site, and
wherein said integrase facilitates recombination between said attA sequence and said attD sequence thereby integrating said donor nucleic acid molecule into said genome of said cell such that said endogenous nucleic acid sequence encoding said polypeptide is interrupted and expression of said polypeptide is reduced or eliminated.

63. The method of claim 62, wherein said nucleic acid cargo comprises a stop codon.

64. The method of claim 62, wherein said nucleic acid cargo comprises a nucleic acid encoding a selectable marker.

65. The method of claim 62, wherein said nucleic acid cargo comprises nucleic acid encoding a detectable label.

66. The method of any one of claims 62-65, wherein said cell is a non-human mammalian cell.

67. The method of claim 62-65, wherein said cell is a plant cell.

68. The method of any one of claims 62-67, wherein said genome-editing system comprises (i) a polypeptide comprising a DNA binding domain and (ii) a nucleic acid comprising a guide sequence that is complementary to said target site within said genome and a sequence that encodes said attA sequence.

69. The method of claim 68, wherein said DNA binding domain is present in a polypeptide selected from a Cas9 polypeptide, a Cas12 polypeptide, a zinc finger polypeptide, and a TALE polypeptide.

70. The method of claim 68, wherein said polypeptide comprising said DNA binding domain comprises a polymerase.

71. The method of claim 70, wherein the polymerase is an RT selected from the group consisting of a M-MLV RT, an AMV RT, and a HIV-1 RT.

72. The method of any one of claims 62-71, wherein said attA sequence comprises any one of SEQ ID NOs:11-84 and SEQ ID NO:254.

73. The method of any one of claims 62-71, wherein said attD sequence comprises of any one of SEQ ID NOs: 159-232.

74. The method of any one of claims 62-71, wherein said integrase is a LSR.

75. The method of claim 74, wherein said LSR comprises an amino acid sequence containing a motif set forth in any one of SEQ ID NOs:233-245.

76. The method of claim 74, wherein said LSR comprises or consists of an amino acid sequence having at least 70% sequence identity to the sequence of any one of SEQ ID NOs:85-158.

77. A method for treating a mammal having a disease or disorder, the method comprising administering to said mammal:
(a) a genome-editing system that can insert an attA sequence into a target site within said genome;
(b) a donor nucleic acid molecule comprising a nucleic acid cargo encoding a therapeutic gene product and a attD sequence; and
(c) an integrase that targets said attA sequence and said attD site;
wherein said genome-editing system integrates said attA sequence into said target site, and
wherein said integrase facilitates recombination between said attA sequence and said attD sequence thereby integrating said donor nucleic acid molecule into said genome of said cell such that said cell produces said therapeutic gene product.

78. The method of claim 77, wherein the therapeutic polypeptide is selected from the group consisting of an adenosine deaminase polypeptide, an α-1 antitrypsin polypeptide, a cystic fibrosis transmembrane conductance regulator (CFTR) polypeptide, a β-hemoglobin (HBB) polypeptide, an oculocutaneous albinism II (OCA2) polypeptide, a Huntingtin (HTT) polypeptide, a dystrophia myotonica-protein kinase (DMPK) polypeptide, a low-density lipoprotein receptor (LDLR) polypeptide, an apolipoprotein B (APOB) polypeptide, a neurofibromin 1 (NF1) polypeptide, a polycystic kidney disease 1 (PKD1) polypeptide, a polycystic kidney disease 2 (PKD2) polypeptide, a coagulation factor VIII (F8) polypeptide, a dystrophin (DMD) polypeptide, a phosphate-regulating endopeptidase homologue X-linked (PHEX) polypeptide, a methyl-CpG-binding protein 2 (MECP2) polypeptide, a ubiquitin-specific peptidase 9Y, Y-linked (USP9Y) polypeptide, a carbamoyl-phosphate synthase 1 (CPS1) polypeptide, an ATP binding cassette subfamily A member 4 (ABCA4) polypeptide, an fatty acid elongase 4 (ELOVL) polypeptide, amyosin VIIA (MY07A) polypeptide, an usher syndrome 1C (USH1C) polypeptide, a cadherin related 23 (CDH23) polypeptide, a protocadherin related 15 (PCDH15) polypeptide, an usher syndrome 1G (USH1G) polypeptide, an usher syndrome 2A (USH2A) polypeptide, an adhesion G protein-coupled receptor V1 (ADGRV1) polypeptide, a whirlin (WHRN) polypeptide, a clarin 1 (CLRN1) polypeptide, a retinitis pigmentosa 1 (RP1) polypeptide, an eyes shut homolog (EYS) polypeptide, a lipoprotein (a) (LPA) polypeptide, a lipoprotein lipase (LPL) polypeptide, an apolipoprotein C2 (APOC2) polypeptide, an apolipoprotein A5 (APOA5) polypeptide, a lipase maturation factor 1 (LMF1) polypeptide, a glycosylphosphatidylinositol anchored high density lipoprotein binding protein 1 (GPIHBP1) polypeptide, a proprotein convertase subtilisin/kexin type 9 (PCSK9) polypeptide, a ryanodine receptor 2 (RYR2) polypeptide, a calsequestrin 2 (CASQ2) polypeptide, a myosin heavy chain 7 (MYH7) polypeptide, a myosin binding protein C3 (MYBPC3) polypeptide, a troponin T2, cardiac type (TNNT2) polypeptide, and a troponin 13, cardiac type (TNNI3) polypeptide, and a C9orf72 polypeptide.

79. The method of any one of claims 77-78, wherein said mammal is a human.

80. The method of any one of claims 77-79, wherein said genome-editing system comprises (i) a polypeptide comprising a DNA binding domain and (ii) a nucleic acid comprising a guide sequence that is complementary to said target site within said genome and a sequence that encodes said attA sequence.

81. The method of claim 80, wherein said DNA binding domain is present in a polypeptide selected from a Cas9 polypeptide, a Cas12 polypeptide, a zinc finger polypeptide, and a TALE polypeptide.

82. The method of claim 80, wherein said polypeptide comprising said DNA binding domain comprises a polymerase.

83. The method of claim 82, wherein the polymerase is an RT selected from the group consisting of a M-MLV RT, an AMV RT, and a HIV-1 RT.

84. The method of any one of claims 77-83, wherein said attA sequence comprises any one of SEQ ID NOs:11-84 and SEQ ID NO:254.

85. The method of any one of claims 77-83, wherein said attD sequence comprises any one of SEQ ID NOs: 159-232.

86. The method of any one of claims 77-83, wherein said integrase is a LSR.

87. The method of claim 86, wherein said LSR comprises an amino acid sequence containing a motif set forth in any one of SEQ ID NOs:233-245.

88. The method of claim 86, wherein said LSR comprises or consists of an amino acid sequence having at least 70% sequence identity to the sequence of any one of SEQ ID NOs:85-158.


SEQUENCE LISTING (254 sequences)

SEQ ID NO: 1 - protein, 1023 aa, Streptococcus pyogenes
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAK

SEQ ID NO: 2 - protein, 1023 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAK

SEQ ID NO: 3 - protein, 1023 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAK

SEQ ID NO: 4 - protein, 1023 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAK

SEQ ID NO: 5 - protein, 1052 aa, synthetic construct
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 6 - protein, 1052 aa, synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - protein, 677 aa, Moloney Murine Leukemia Virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 8 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 9 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 10 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 11 - DNA, 110 bp, synthetic construct
  source 1..110: mol_type = other DNA
gtggtgcttgttacataactcttatgtatattttatatgggggttatagggggtacgtagcaccggtaccccccatagttgcaacgaacatttgtgtaccaggtgtaata

SEQ ID NO: 12 - DNA, 112 bp, synthetic construct
  source 1..112: mol_type = other DNA
ttccgacgcagtttccgacgagtacgaggacgaggacagacgtgcctaccggcaaggtcaagtggttcaacagcgagaagggcttcggctttctctcccgcgacgacggcgg

SEQ ID NO: 13 - DNA, 118 bp, synthetic construct
  source 1..118: mol_type = other DNA
gcgtcagatcgacgatcgtcggcagcagcgcgagatagaacagcatgatcttcgggttgccgagcgtgaccagcgtgccggcgacgaacatccgtatcggcgattgcccgcgcggcag

SEQ ID NO: 14 - DNA, 109 bp, synthetic construct
  source 1..109: mol_type = other DNA
cggctgacgctggcgctgtcgccggccacgctgccgaagatggggtcggtctacgatctcgcgctggcgctggccgtgctgtcggcggagaagaaaaccgagtggcccc

SEQ ID NO: 15 - DNA, 114 bp, synthetic construct
  source 1..114: mol_type = other DNA
ccggaaaggtccggatggaaggcaaggaatacgtcatggttgacggtgacgtggtggaattccggtttaacgtctaggcggaagcgctactaatgccgcgccgccgcgaacttc

SEQ ID NO: 16 - DNA, 117 bp, synthetic construct
  source 1..117: mol_type = other DNA
gctggtcgaggagttaatgaagatctccaacatgttcggctacggtgaggtaaatcagtacaatcgtcactcgaccctaatgtctgcctataaaaacatcaaagatgaacatttccg

SEQ ID NO: 17 - DNA, 105 bp, synthetic construct
  source 1..105: mol_type = other DNA
tcgtcgacgcctcgctgcccggcggcgagcggctccacgtggtgatccccgacgtcacgcgcaccgcgtgggcggtcaacatccgcaagcacgtggtccgtgcgt

SEQ ID NO: 18 - DNA, 111 bp, synthetic construct
  source 1..111: mol_type = other DNA
tcaaacgctaggtcttctgcttctagccattttttggcttttttaattgtgtcgcaatttgggatgccgaacatggtaatcgtcatattatttccttttctttttattttt

SEQ ID NO: 19 - DNA, 114 bp, synthetic construct
  source 1..114: mol_type = other DNA
agaatggcgcccattttctttgaatcacagaatagcaaattatgagcgtacgtttagtgttagccaaagggcgcgagaaatctctcctgcgccgccatccctgggtcttttccg

SEQ ID NO: 20 - DNA, 112 bp, synthetic construct
  source 1..112: mol_type = other DNA
cttgaccgaatgggacgaagtagatgttttttgtggccattaggcgcatgaggttgacgccattaagccctaaagcatcattcgtcgaaacggccaatacgacaggcttgcc

SEQ ID NO: 21 - DNA, 115 bp, synthetic construct
  source 1..115: mol_type = other DNA
gtgacccgttccgccgtcgagaacgccacctccgtggcgcgcatggtgctcaccaccgagagcgccgtggtcgacaagccggccgaggaagagcccgcgaacggccaccacggcc

SEQ ID NO: 22 - DNA, 111 bp, synthetic construct
  source 1..111: mol_type = other DNA
tagcacctatcttattggcattgattggtgcgttaaatactccacctgtagtagatacgtgtgtcccaataaatttatttatctttctattttccattaaataattctcct

SEQ ID NO: 23 - DNA, 117 bp, synthetic construct
  source 1..117: mol_type = other DNA
tgccgccacgccccacattcaagatgtcccaatcccccaaagtagggttcgtatccctcgggtgcccgaaagcgctggtcgactccgaacaaatcatcacccagctgcgcgccgagg

SEQ ID NO: 24 - DNA, 110 bp, synthetic construct
  source 1..110: mol_type = other DNA
ggctggccggtacgtcccggccccctgcggaacatctgcacggcgcacgccaagtcgtaggtgatcacgccgtcgagcgccagcgctgccaccgtcttggtgcccatacc

SEQ ID NO: 25 - DNA, 116 bp, synthetic construct
  source 1..116: mol_type = other DNA
gctcggcggacggcgacccgtacctggcaagctgaacgccgtgttccgagtgatgttccacgagccccgcatcccacccaatacgggaaacgcgatccgcatggtcgccgggacgg

SEQ ID NO: 26 - DNA, 119 bp, synthetic construct
  source 1..119: mol_type = other DNA
gttttttcatataaagttacatcagcacctgcctttttagctgttatggctgctgcacatccagaccagccacctccaattattataatcttcatattagtcttctcctttcaaaaaca

SEQ ID NO: 27 - DNA, 112 bp, synthetic construct
  source 1..112: mol_type = other DNA
acgtagtagacatttttctcgtccaggcggtccttggcgaggcgcaggccttcgccgaagcgctggaagtgtacgatctcgcgcgcacgcaggaacttgatgacgtcgctca

SEQ ID NO: 28 - DNA, 113 bp, synthetic construct
  source 1..113: mol_type = other DNA
catgaatgcagaccgaaagtaacgtcggccaggggaagcggcgaggtaaatcaagggtcattgaagtcataatcatttcagtagaaaaaccaggtctcgattttaaatgcaaa

SEQ ID NO: 29 - DNA, 107 bp, synthetic construct
  source 1..107: mol_type = other DNA
ctacggaatagagataacacgaggagtggttagaaatggctaaagttctggtgctttattattccatgtacggacatattgaaacgatggcacgcgcagtcgctgag

SEQ ID NO: 30 - DNA, 104 bp, synthetic construct
  source 1..104: mol_type = other DNA
caaatttgacggaaatgttttcaaacaacggcttactgccgaactgcatggtgacgttactggaaactaacacaggcgtatcctgaaaagagatatgacaaacc

SEQ ID NO: 31 - DNA, 117 bp, synthetic construct
  source 1..117: mol_type = other DNA
gcaccatgaatgcagaccgaaagtaacgtcggccaggggaagcggcgaggtaaatcaagggtcattgaagtcataatcatttcagtagaaaaaccaggtctcgattttaaatgcaaa

SEQ ID NO: 32 - DNA, 104 bp, synthetic construct
  source 1..104: mol_type = other DNA
gcttggcgttaccggtcactccgccctgaatatgtggtccggtattgtcttcagcattacatttttattttcggccatcgcctcaccgttttggggtggactcg

SEQ ID NO: 33 - DNA, 105 bp, synthetic construct
  source 1..105: mol_type = other DNA
cgacaccaactggcttggcttctgcttggattttacgccatccagccaatatgcaagtgatcgccggtacgatgaacgtagggcgaatcaaggaaatcgctcaag

SEQ ID NO: 34 - DNA, 107 bp, synthetic construct
  source 1..107: mol_type = other DNA
gtatccttttggtaaaattcatatcctgctgcgatggaataacattaccagaaggatgattatgcgctaaaacgatccttgctgcacaataacgtacagcataatgg

SEQ ID NO: 35 - DNA, 111 bp, synthetic construct
  source 1..111: mol_type = other DNA
tccgggggcccccactattcatatgaacggctctcaacctgtgctaaaaaacgaaaggacggcatgccatgaatatattcgatcactatcgccagcgctacgaagctgcca

SEQ ID NO: 36 - DNA, 113 bp, synthetic construct
  source 1..113: mol_type = other DNA
tttgcatttaaaatcggagcatcatttttcaacagaaacgactatgagcgcaatgacccttgatttacctcgccgctttccgtggccaacgctgctgtccgtggctatccacg

SEQ ID NO: 37 - DNA, 105 bp, synthetic construct
  source 1..105: mol_type = other DNA
cgatcatcgccggactggtggccgcagcgctatggaccgggcggctgtcgaggatcacgcggttgacgttacgctcgtgcagcccgcggttgagctgctgttccg

SEQ ID NO: 38 - DNA, 108 bp, synthetic construct
  source 1..108: mol_type = other DNA
cgaggatgagttatgaagctggaagaaatcgtagcccttagtgtaaagcataatgtctctgatctacacctgtgcaattccgccgcaccacgctggcggcggcagggc

SEQ ID NO: 39 - DNA, 104 bp, synthetic construct
  source 1..104: mol_type = other DNA
ccggtttcccttcgcacccgcaccgcggcttcgagaccgtgacctacatgctcgaagggcgtatgcgccacgaagaccacctcggcaatcgcggcctgctcaag

SEQ ID NO: 40 - DNA, 102 bp, synthetic construct
  source 1..102: mol_type = other DNA
aactgccggagttcgagcgcaaggtcctggaggtcctgcgcgagccgctggaaagcggcgagatcgtcattgcccgggccaacggccgggtacgtttcccgg

SEQ ID NO: 41 - DNA, 109 bp, synthetic construct
  source 1..109: mol_type = other DNA
tgattgttttaagtgggactttttatattgcaaaaaataaatggcggacgaggtaacaggatacctcatctgccaattaaaatttgttaatttaataattaaataaaaa

SEQ ID NO: 42 - DNA, 106 bp, synthetic construct
  source 1..106: mol_type = other DNA
aacatgaggttattgttgctaatattaataagttatattggaggaacgtgtgcgttagaagtcgtaccattcatgtccttacgagataaattaactaaacacgtat

SEQ ID NO: 43 - DNA, 108 bp, synthetic construct
  source 1..108: mol_type = other DNA
gtggcaaaccttttcagtgcgtgattggcaccggccgggtgatcaagggctgggatcaagggctgatggggatgcaagtgggcggcaagcgcaaactgctggtgccgg

SEQ ID NO: 44 - DNA, 108 bp, synthetic construct
  source 1..108: mol_type = other DNA
ataatgtactggttaaaagtaatttatgagcaatatataaaaaataatactaaaagtaattaatttttatatattgctcatatttaaaaaaaaatataaatataagct

SEQ ID NO: 45 - DNA, 102 bp, synthetic construct
  source 1..102: mol_type = other DNA
gacagtatcaaaattttatggaaaatttaacaaatttagtatcattcatttcaatcaaatatattaaatttgtttattaaataggaagaaaaagacggtcat

SEQ ID NO: 46 - DNA, 106 bp, synthetic construct
  source 1..106: mol_type = other DNA
taggaaggaatttttttataatacataattacatacaacatatagtatgtaatgaataacactcaatatgatgtatgtaataaatccaaagcttagtaattagaat

SEQ ID NO: 47 - DNA, 104 bp, synthetic construct
  source 1..104: mol_type = other DNA
ccaagcaatactatagcttcaggtaaataggaactttaatacatttatctgaagatatatgtattaaagtaaaactttttaattatgatgtcaattaattctta

SEQ ID NO: 48 - DNA, 129 bp, synthetic construct
  source 1..129: mol_type = other DNA
acgtcaattaagttttcgtgttttttattgaatagccttcttagtagtttcatttgtagttcctccttcattcgaaatcttcaattgacaaggtttcaattcgtttttggtaacgatataaataaaagt

SEQ ID NO: 49 - DNA, 133 bp, synthetic construct
  source 1..133: mol_type = other DNA
taatttaacaaggcagataatttaaccgcaggggacgcaaaggacgctaaattttttttataatttactatttttttcaaataattcttataaatataatggggatgggaaaatattaaaaaataataggaga

SEQ ID NO: 50 - DNA, 102 bp, synthetic construct
  source 1..102: mol_type = other DNA
aaggaagggacgtgctgggaatcatgcccactggggccggaaaatccctttgttatcaaatccctgcccttatgatggatggaatcacgctggtcatttccc

SEQ ID NO: 51 - DNA, 105 bp, synthetic construct
  source 1..105: mol_type = other DNA
ggagaatagcgggatcgaaccgctgacctcttgcatgccatgcaagcgctctcccagctgagctaatcccccacatattcggttggtgtcctgcgacgtgagtta

SEQ ID NO: 52 - DNA, 105 bp, synthetic construct
  source 1..105: mol_type = other DNA
aatcacgatctattggtctcaattctccattcgtgattgtatggttattgttgaataagttaacaatcgcgaatttatgaaaattgcctatgcccgtgtctcaag

SEQ ID NO: 53 - DNA, 102 bp, synthetic construct
  source 1..102: mol_type = other DNA
cattcattgcagatgtatgagatggaaaaaagaaataattttactatcctttgtggaaatgtaggttactaaaattacttatattttccacttgatgacaat

SEQ ID NO: 54 - DNA, 105 bp, synthetic construct
  source 1..105: mol_type = other DNA
gtggcaaacctttccagtgcgtgattggcactggccgcgtgatcaagggctgggatcaggggctgatgggcatgcaggtgggcggtaaacgcaaactgctggtgc

SEQ ID NO: 55 - DNA, 102 bp, synthetic construct
  source 1..102: mol_type = other DNA
tgcgtctaatctacggttataagattttttgtgtttatgttatgtttacatgcttaaacctgacataaatactaataaaattctatatgagtgattattatt

SEQ ID NO: 56 - DNA, 106 bp, synthetic construct
  source 1..106: mol_type = other DNA
cgggcagggtctacctaagcctttacatttgtgtacatctgaaattgttgcttgtaggtatctcatatgtttacaatttgcacccaagattctttcagagggcgcc

SEQ ID NO: 57 - DNA, 148 bp, synthetic construct
  source 1..148: mol_type = other DNA
agcggcacagaaaccaagcgacgaattcatcaagaagataacttgaaagaaatggtgcccggaggcggatttgaaccaccgacacgcggattttcaatccgctgctctaccaactgagctatccgggcacttcaggtccttgaagaac

SEQ ID NO: 58 - DNA, 128 bp, synthetic construct
  source 1..128: mol_type = other DNA
ataaatttctgtagttatttttcaaaaaccgcatcattaactgataagcagaagcatatcacaaataaaactaaaaaaacgatgttgaacaataatattcattatgaattttttgagtaaatcttagg

SEQ ID NO: 59 - DNA, 103 bp, synthetic construct
  source 1..103: mol_type = other DNA
acgctgtgctcttttgttttgtaatttttcgtatttacgtgaactttatatgtgtaaatgtaacataaacactaataaaattctatatctaatacttctgtaa

SEQ ID NO: 60 - DNA, 105 bp, synthetic construct
  source 1..105: mol_type = other DNA
aataattttaattttttataaaaaatattcatatattctttatattaaagtttagatatctaaaaatacttttagaatttattatattatgttaatttttttata

SEQ ID NO: 61 - DNA, 102 bp, synthetic construct
  source 1..102: mol_type = other DNA
tgtctgaaataacagacactaaatatataagtgttttatgtacatttattgaaataagtgtaagttaaacactctattttttaaataaaatttccatgtcct

SEQ ID NO: 62 - DNA, 106 bp, synthetic construct
  source 1..106: mol_type = other DNA
gccggcaccagcagcttgcgcttgccgccgacctgcatgcccatcaggccctggtcccagcccttgattacccgaccggtgccgatcacgcactgaaacgccttgc

SEQ ID NO: 63 - DNA, 110 bp, synthetic construct
  source 1..110: mol_type = other DNA
tgaaaagctattttatacaacgggggcatagctcagttggttagagcatctgactcttaatcagagggtctagggttcgaatccctatgcccccattgggtgccaaaccc

SEQ ID NO: 64 - DNA, 102 bp, synthetic construct
  source 1..102: mol_type = other DNA
gcacgaacaagcgacgcaccccgcccacctgcatgcccatcaggccctggtcccagcccttgatcacgcgccctgtgccgatcacgcactggaaaggcttgc

SEQ ID NO: 65 - DNA, 105 bp, synthetic construct
  source 1..105: mol_type = other DNA
gtggcaagccatttcagtgtgtgatcggcaccggtcgcgtcatcaagggctgggaccagggcctgatgggcatgaaagtcggcggcaagcgtcaattgttcgtcc

SEQ ID NO: 66 - DNA, 110 bp, synthetic construct
  source 1..110: mol_type = other DNA
tcagcagcgcggccacctgctcgtcggcgaaggactcgtgcgccatgacgtgacaccagtggcaggcggcgtaaccgaccgaaatcatgatcgggacgtctcggcggcgg

SEQ ID NO: 67 - DNA, 113 bp, synthetic construct
  source 1..113: mol_type = other DNA
aagttaaagcggaggtttctctgtacgaccccattggtgtagacaaggaaggtaatgaaataagtttgatagatattttgggtaccgacccggaagtggtggcggacatggtg

SEQ ID NO: 68 - DNA, 102 bp, synthetic construct
  source 1..102: mol_type = other DNA
actccaagcaggtaggccgtttttctaaactatgctaagcaatttctttaattaaagtttttgctttgtatggtttaggtgataagctctgcacccctttaa

SEQ ID NO: 69 - DNA, 103 bp, synthetic construct
  source 1..103: mol_type = other DNA
tggaatcggtgggaatgaaaatcaccgttaattatggagaaaaggatgggaaaattgtcaagggttctaagacctacggcgatgtcaagaaagaagtgacaga

SEQ ID NO: 70 - DNA, 105 bp, synthetic construct
  source 1..105: mol_type = other DNA
cgaaaaaaaggaatacctctataaaataatatgggtattctctaatatttatttctataaatatagagaaatacccatattttcgcatataaaagaataaattat

SEQ ID NO: 71 - DNA, 103 bp, synthetic construct
  source 1..103: mol_type = other DNA
tgtcttgtaagcacccatccctgtagttatgcactgtacatttgctttcagtctaatgtatagtgataacttacattttatggtatggtgtaacaatgagaaa

SEQ ID NO: 72 - DNA, 147 bp, synthetic construct
  source 1..147: mol_type = other DNA
tgtcgctgcatcgaagcgggcccctgtagctcaattggatagagcatcggacttctaatccgacggttgcaggttcgagtcctgccgggggcacttccaggacgctgtttggcacaccagcgtccttctcggtagcgaccgccatga

SEQ ID NO: 73 - DNA, 106 bp, synthetic construct
  source 1..106: mol_type = other DNA
atatgaatctatccttagtagctattgatgaggctcattgtatttcgcagtggggacatgattttcggccaagctatttaaaaattaaaacccttttaaaagagtt

SEQ ID NO: 74 - DNA, 106 bp, synthetic construct
  source 1..106: mol_type = other DNA
tattacgctgatttacagctgattatttaatcaataatgtttttagtttaatcaattaaactaataacttttatgattaaatttaaagtgctatatttgtgctgaa

SEQ ID NO: 75 - DNA, 131 bp, synthetic construct
  source 1..131: mol_type = other DNA
ccaaaatgatttttgtttcattctaattttccaattaatcatattcttatctccttttatccaaaataaaaaacgactaaaaaattagtcgtttaaaattattcaatggtcaatgttggagatcctgaata

SEQ ID NO: 76 - DNA, 103 bp, synthetic construct
  source 1..103: mol_type = other DNA
cgtaggccgcgagctcctcctcgacgacagggcgcgggccgatcacgctcatctggccggccagcacgttgaggaactgagggagctcgtcgagggaggtctt

SEQ ID NO: 77 - DNA, 104 bp, synthetic construct
  source 1..104: mol_type = other DNA
ataatttatatattaataaaattatactcttaaaacaaaaaagaagctctaaaaagagcttctttttataattaaatttatattattttaatctttcttctaac

SEQ ID NO: 78 - DNA, 114 bp, synthetic construct
  source 1..114: mol_type = other DNA
ttgcccggtatcaggaaactttacttatgatagttgacactgcttattcaacacatgtggagtgtgtggtgttgttggttaggaatgacagataattgaggtatactgacgtgt

SEQ ID NO: 79 - DNA, 104 bp, synthetic construct
  source 1..104: mol_type = other DNA
ataaatttactgaggctttatcggttaaggttccttgaaaaaacaacaaaatcaggttaaattaggttttttaaggcagatgcaatctgtttgtgctgccaata

SEQ ID NO: 80 - DNA, 106 bp, synthetic construct
  source 1..106: mol_type = other DNA
aagtaggcggtcatgggttgtatttatcaatattttaaaattagtgaaactccactttcccttagttaaagatatgaaccctatttattagtctactttcccaata

SEQ ID NO: 81 - DNA, 122 bp, synthetic construct
  source 1..122: mol_type = other DNA
cctgtgatatatgaattaatatcttattcccttcagcatatccaaacatctcatttatatatttaaatttatcaatatctatatataataaagcatactttacttgagtatttttacatagt

SEQ ID NO: 82 - DNA, 102 bp, synthetic construct
  source 1..102: mol_type = other DNA
ctcctgttgctcctgttgggcctgttactccatctgctcctgttgcccctgttgggcctgttgggcctgttactcctgttgctccatctgctcctgttgctc

SEQ ID NO: 83 - DNA, 102 bp, synthetic construct
  source 1..102: mol_type = other DNA
catatgagtcaagcaaataaagtgtatctcatttttaaacgaaacatgtattacatttaaaaataggaattttaccccatatgatattatacattaactaag

SEQ ID NO: 84 - DNA, 112 bp, synthetic construct
  source 1..112: mol_type = other DNA
gaattttatatattttaaatatgatagtttaaaattatgaaagaatgataaattccagtttgtagggtaattttgataaataatattaataagtatgagtaaaaagtaggag

SEQ ID NO: 85 - protein, 465 aa, synthetic construct
MKVAIYTRVSSYEQATEGYSIHEQERKLKAFCEVQNWNEFKVFTDAGVSGGSMNRPALKRIMDNLEYYDLVLVYKLDRLTRNVKDLLEMLETFEKYNVAFKSATEVFDTTTAIGKLFITMVGAMAEWERATIRERALFGSRAAVREGNYIREAPFCYDNVDGKLVPNKYKWIIDYLVEQFKHGVSGNEIARQMNVKKVNVPKVKKWNRTSIIRLMKNPVLRGHTKYGDMYIENTHEPVLSESDYKRIIDVIENKTHRSKVKHHAIFRGVLTCPQCHNKLHLYAGKITDKKGYSYEVRRYKCDTCSKDKNVQTISFNESEVEDKFIELLKTYDMNKFKVDIVEESTPKLDYDIDKIMKQREKLTRSWSLGYIEDDEYFSLMDETKEILDEVERAGTEVESTQTVTNEQLNMIDNILIKGWSKLNVEQKEELILSTVKEIVFDFVPRKYNENGKVNTLNIREITFKF

SEQ ID NO: 86 - protein, 555 aa, synthetic construct
MQPNLRYLACLRLSADSDGSTSIEWQRGVIRHHVSSPHLSGVLVGEAEDTDVSGSLSPFKRPKLGKWLTAKADEFDVIIAAKMDRLTRRSMHFNELLEWAQQNGKFIVCVEEGFDLSTPQGKMMARMTAVFAEAEWDTIQARILNGVQTRLENRSWLVGAPPTGYRIKTVEGGKRKILEIDQDFYPYVEEIFRRIREGQSTHRIARDFNGRSVLTWGDHLRKLKGEEPKGTQWQATIINKFIRSSWVPGLYTYKGEAVLDDQGDPVILPETPLATMDEWTDLVDRIKPAPKPEGATGGSRNSAKSLLSGVAHCGECGAPFTSLMDSGYKRKDGTKVPGHRRYRCSNKFKGGDCKNGSYVRADVLDSWVDQAIRDSIGQEDMYERAGKGPSQARELQETKARLAKLEADYESGKYDGEGQDESYWRMNKNLSAKVAHLAKQEAERANPTFKATGKKYGEVWEAKDQEDRRDFLRTYGVKVFVWGEGADKKDRGYAMNLGDIKTMAEELFPNRDRARFKLVHTHNAPEGYLSKIGIAVGLLKYGHPLEVKLRSPENS

SEQ ID NO: 87 - protein, 560 aa, synthetic construct
MAKKPKAKVYSYLRFSDPKQAAGSSADRQMEYAARWATEHDMQLDATLTLRDEGLSAFHQRHIKQGALGVFLRAIEDGRIQPGSVLIVEGLDRLSRAEPIQAQAQLAQIINAGITVVTASDGREYNRERLKAQPMDLVYSLLVMIRAHEESDTKSKRVKAAIRRQCEGWVAGTWRGIIRNGKDPHWVRLGEHGKFEHVPERVLAVRTMIDLFLEGHGAIEITRRLTEQNLYVSNAGNYSVHMYRIVRNQALIGEKRISVDGEEFRLDGYYPPILTREEFAELQQTMSERGRRKGKGEIPNIITGLSITVCGYCGRAMTTQNSKARAPKGKSVVRRLSCPMNSFNEGCPIGGSCESEIVERALMRYCSDQFNLSRLLEGDDGTARRTAQLAVARQRASDIEAQIQRVTDALLSDDGKAPAAFTRRARELETQLEEQRREIEALEHQIAASSAHGIPAAAEAWAQLVDGVLALDYDARMKARQLVADTFRKIVVYQRGFAPIDDAAADRWKRSGTIGLMLVTKRGGMRLLNVDRRTGCWQAEDDLDPSLIPSDGLPMLPLDA

SEQ ID NO: 88 - protein, 476 aa, synthetic construct
MKAVIYTRISKDREGAGLGVERQRADCAALADRLGWQVVGTFSDNDLSAYSGRHRPGYAAMCEALEAGEAQAIIAWHTDRLHRRPVELESFIGLCERRSIQVRTVRAGTLDLSTPSGQMVARMLGAAARHEIDHAIERQKRAKKQAALDGRYRGSRRAFGYERDGLTLCDAEADAIRTAAERVLSGTSLSQVARDWNAAGLRTAFGGKAFTSREVRRILLRPRNAGYSLHEGKRIPNAQWPPIITTDTFAALEALLRDPVRSKHLAFERKWMGSGVYLCGKCGAKISTASQKGTGKSWRPTYVCSASKHLGRVADTVDEYVTEAVLERLSRPDAPILLGGNKVDVADLTSRRDGYRARLDELAAMFAAGDIDAGQLKSGTTELRRKLDRVDAELAAARASSVLADLVLSGDDLRDTWAAIPPGGKGKVIDALMTVTIEPTRRGRRPGGSYFDPESVTIRFKGVGEHRLDDGQLIGA

SEQ ID NO: 89 - protein, 455 aa, synthetic construct
MASPPRNAALYLRISLDQTGEGLAIDRQREECERIAAQRGWTVVGVYEDRSISATQANKKRPGYEQLVSAYQAGQFDAMVCYDLDRLTRQPRQLEDWIEAAEGRGLALVTANGEADLTTDGGRMFARVKLAVARSEVERKSARQRTAAHQRASLGRPPLGTRLTGYTPKGETIPAEAEVIRRIFKLFQAGETLRSITRMLTEEGVTTRRGNAWNPSTIYGTLTNPRYAGRAVYQGKPTGQLGNWEPLVSPEVFDLIQARLADPRRKTNRVGTDRKHLGSGLYVCAVCEQPTTSWSQGRYRCKDSHVNRAQSQVDSYVLDTIAARLRRGDIATLLAPAKADLAPLLDDIERLTARQATIDADYDAGLIDGTRHAAATATVRAELIAVQQQMAAADKGSALGELLTSPDPAQAFLDAGLMTQRSAIDALAVVRLHRGHRYSRTFDPETVEVDWRRPR

SEQ ID NO: 90 - protein, 515 aa, synthetic construct
MSRPTGLTIDIYLRKSRKDLEEEKKASESGETYDTLERHRRTLFAVAKKERHNIANIYEEVVSGESVSERPQIQAMLRNLETSHIEGVLVMDLDRLGRGDMLDQGLLDRAFRYSGAKILTPTEVYDPESETWELVFGIKSLVAREELKAITRRMQRGRVASAGEGKSISKVPPYGYLRDENLRLYPDPETAWVVKKMFEMMRDGHGRIAVAQELDKLGIKPPNDKRRSWAPSSITAIIKNEVYLGTIIWGQVKYSKRNGKYKKTKLPRSKWTIKENAHEPIVSRELFEAANRAHTGRWRPSTNATKTLSNPLAGVLKCDVCGFTMLYQPRPNRPNDFIRCTQPTCQAVQKGATLALVEQHILDGLKQFAQELELQTEVPELDNDKDIAVKKSLVGNKQEEIAQLETQKSKLHDLLERGIYDVDTFLERQQNLNNRINGLQDDIRNIESEIKKEEVRNSSVLNLLPQLQTVISEYENADTESKNRLLKSVLEKATYLRKKEWTKRDQFIIQLYPKI

SEQ ID NO: 91 - protein, 146 aa, synthetic construct
MSRRRALAPVPDTPPRAVAYLRQSTYREESISLELQETACRDYAARHGYQIVAVEKDPGISGRTWNRPAVQRVMEMIESRDADVIVLWRWSRLSRNRKDWAIAADRVDVAGGRIESATEPNDTATAAGRFARGVMTELAAFESERI

SEQ ID NO: 92 - protein, 542 aa, synthetic construct
MKTAYSYIRFSSSRQADGDSIRRQTELARAYAEEHELDLQDVSISDFGVSAFRGSNATEGALGAFLKLVDEQKIDSDCYLLVESFDRLSRQAVEDALALLLQVVNRGITLVTLSDNHVYKRGELDMTMLILSIVTMSRANEESEIKSQRTSAAWSKARIDALNGIKVKNSRLPDWLSWNEDKTDFVVNQPAKATIQRMFELSAGGSGIEIIAKTLNSEGLKTFKQFKEWRSSGVSKLLRNRAIVGEFQFYRKDAKGNRVAVGEPIAGYYPEVVSKQLFLTVQQGMDLRNRRGSGNRAGQFTNLFTGLIRCGECGSAVVTSSQTTKTPQGYLKCTMRCESKARMNYKYTEPQVLSALGSLQSVIEKYRTPISDETASIELDVRALDEKVTTFESLLDNAFTPKLAERLQEAELQLANAKALLESERQRVSAEQSREQIVLGLEPLESTDDRRAFNSKLRTVIDRIEMIEHPHEKGSALVYFINDCPVMEQHFTKLKGAHGTSSEVYELHSDDVPSHLGRTTSKKPLEFIIEESGDVLSFMPAQ

SEQ ID NO: 93 - protein, 562 aa, synthetic construct
MEKRKLYSYIRWSSDKQAKGSSLQRQLETARRVAHENDLELVEIIDAGLSAFRSKHLEKGSLGAFIEAVKVGQIASDSWLTVESLDRISRDAILKAQGLFMELLELGITIYTGMDNRIYTKSSVTDNPMELMLSIMTFSRANQESMVKSQRQKSATQLKINKFNASVRADNGYPHAIRNSGANVFWSDVSDGTVRPHEYYFPIAKLIVSLRLKGWGYMRIAKHLNENYTPPKGTAKRKHFKDLWSPKLICNFLQSRTLMGEKSIRVDGVEHILKGYYPQVISENEFYSLQNVINRKLANEPNNYIPLLTGIRRFKCGCGAAMVAFFQYDKIRYRCDGMKSLDKRMHCGAKSFNGASIENAIVQICADKIFKDVKTHTSNVGAIQAMLVEAKRKYNRGMDMLLEDTAPQGLNIKLIELEKQIEELQKKLNDELVTEAGVNSSVSWGAVPKDYRDVFNTERLEIKQKIQASTLSIVCTTIKTKGHLFEIKFNDGDVIRFFSDKRRVHVDVHSINNAEIMQSEGIILHDHLDYLVGPEEFIERIRQKHLQMKALQERTLDDIFDI

SEQ ID NO: 94 - protein, 515 aa, synthetic construct
MKTAIYLRKSRADLEAEARGEGETLAKHRSTLLKIAKEMNLNVLSVREEIVSGESLVKRPEMLALLEEIEDNKYDVVLCMDMDRLGRGGMKEQGIILETFKRSNTKIMTPRKTYDLNDEWDEEYSEFEAFMARKELKIITRRMQRGRVASVEAGNYLGTHAPFGYDIHRLNKRERTLTINPEEASIVRMIFDWYANEDMGANAIRSKLNDLGYKSKLGNEWNPYSILDILKNNVYIGKVTWQKRKEVKRPDAVKRSCARQDKSDWIIADGKHEPIIPESLFEQVQEKLNSRYHIPYNTNGIKNPLAGIIKCAKCGYSMVQRYPKNRKETMDCKHRGCENKSSYTELIEKRLLEALKEWYINYKADFEKHKQDDKLKETQVIQMNEAALRKLEKELVDVQKQKNNLHDLLERGVYTVDMFLERSNVVSDRITEITSTMENLKKEIKTEIKKEKVKKDTIPQVEHVLDLYFKTDDPKKKNSLLKSVLEKAVYKKEKWQRLDDFELVLYPKLPQDGDI

SEQ ID NO: 95 - protein, 605 aa, synthetic construct
MPESPPRALIVIRLSKVTDATTSPERQLAECRAICEKRGYEVVGVAEDLDVSGAVDPFDRKKRPHLARWLHGEHLDDNGEPVPFEVIVVYRVDRLTRSVRHLQKLVAWADDHKKLVVSATEAHFDTSLPFSAVLIALIGTVAEMELAGISERNASAARHNIQAGRYRGSTPPWGYVPSNDTGEWRLVRDKEQADVINEVARRVIEGEPLQKIAHDLTRRGILTPKDNFAKQRGREIKGREWSVTQLKRSLLSEAMLGHAVSGGAAVRNDDGSPVVRSEPILSREIYDRVAAELSSRAKRGEPNKRTSSLLLGVISCGNPCLHKQQGHECPEGCSGTCDEPVYKFNGGSHSQFPRYRCRTMTRAYKCGNRTIRADQADAQVERTILALLGSSERLERVWDAGEDHSAELADINDELVDLTSQLGVGAFRAGTPQRAKLDARIASLAERQAQLSSEAVVPAGWKLLPTGELFGDWWSRQDLTARNVWLRSMGVRARFKRDDKTLYIDLGNLNDLISGLKPGGTAQRVRGGLQAMERNGIQGMVFSDADSEVMPAPAAGYMWIQPVEGVWVYTSEALLAAAAERQALRREKIEDEFAYGPGDFDEDWD

SEQ ID NO: 96 - protein, 477 aa, synthetic construct
MRDSKSKTVIIYNRVSTIMQDKNESLTEQTDECIRYCKTNGYEIYKILKDVKSGTKDDRAGYLELKKHIRRRDFDILVVLETSRIARKMKELVLFFSLLNENNIEYISIREPNYNTTTPDGKFAMNIRLGLIQFERDNTAERVTDRLYFKASKGQWVNGKPPIGYKLVNKRLEIDEEKAEIVKNIYEDFLNGYSLNQINNKLQFSWGSKQVKRILTNPTYKGYIRYGTRSNRKKNNREAFIVKGWHEAIIPEDKWEKVQEMYKSLNRKASNTKPTLLSGLLKCKECGNNYIRKRGGSYDKNLYYGCNLNNLRYSDKFFYKDIPECSSATIKGDLLEKAVIDTLKKQINDLNFNDIEIDKKRQVNIKQIDSSINKFKNRLNKIYELYIEDEIPKDKYLKDKKDIEARIISLEKQKKSFGEIEVEKSNNEMIQEYFSKIDLSNIEEANRILKIIVNKIVVYKKKKTPKDDFEVEIYLNI

SEQ ID NO: 97 - protein, 550 aa, synthetic construct
MAAKSRVYSYLRFSDPKQAAGGSIDRQLEYAARWAADREMELDASLSLRDEGLSAYHQRHVRQGALGVFLRAIEDGQVPAGSVLVVEGLDRLSRAEPLQAQAQLAQIVNAGITVVTASDGREYNRERLKAQPMDLVYSLLVMIRAHEESDTKSKRVKAAIRRQCEGWIAGTWRAPIRVGKDPHWVRETEGGGFELAADRASAVRLVIEMFRQGHGAVRIVRELAERGLQISETGRTHSSNIYKILANRMLIGEKSVEVDGETYQLDNYYPALLTPAEFADLRYLAEQRGRRKGKGEIPGVVTGLGITYCGYCSAAIVAQNLMGRKRLPDGRPYPGHRRLHCVTYSQSVGCKISGSCSVVPVERALMLYCSDQMNLTRLLEGDAGTASISAQLASARQRVAELEAQIRRVTDALLFDDGDAPAAVLRRVRELESQLASERRDVESFEHHLAASASNVAPAAADAWRELVQGVEQLDYDARLMARQLVADTFSRIVIYQSGFQPETDHGTIGVVLVGKRGNTRMLNVDRRTGEWRSAEDIELSGLATIPLPA

SEQ ID NO: 98 - protein, 472 aa, synthetic construct
MRVLGRVRLSRLTEESTSVERQRELIKKWSEMNDHTIVGWAEDVDVSGSIDPFEAPELGPWFQEDKRGDWDILVAWKLDRIGRRAIPLNKVFGWMLEHEKTLVCVSDNIDLSTWVGRLVANVIAGVAEGELEAIRERTKASRKKLLESGRWTGGPVPYWLIPEKLPEGGWVLSLNTETAPILRRAIDEVLDGTAVHTVAERLNDQGVPSPGGKKWTSQTLWRILQHKYLKGHSTDRGKTVRDSSGVPISNCEALLTPSEWDRLQAVLTQWKLPETSNRVKNTSPLLGVVVCYICDKPLYYRSYTRNYGKGLYRSYYCRNHRTPGIKADMLDEYLEENLMREVGDKNVLERYFVPAENHQIELDEAIRATEELTALLGTMTSATMRSSLTAQLAALDSRIASLEKLPTSESRWEYRELPRTYREMWESDDDPQFRRELLLKSGITLAATMTGGQKLHLHIPDDILERMALKGE

SEQ ID NO: 99 - protein, 612 aa, synthetic construct
MESASPGLRVLGRLRLSRLTDESTSIARQRETIQRWAEIQGHTVIGWAEDADVSGAVDPFDTPQLGQWLNHRVEDYDVIAAWKLDRLGRNAIQLNKLFGWCIDHNKTLVSCSESIDLGSWSGRMLAGVIAGLAEGELEAIRERARSSRVKLREAARFAGGKPPFGYRKVRRDGGGWMLEIDEPAAELVGKIVADVLDGKPVSRVVMALNEGGYRTPRDYYETVKAGKPALKLAAGERRNSEWRSTALRNLLTNPALRGYVHHKGQIARGDDGMPLQLAEEPLVDADEWEGLQAIFNGHRERRSHYRRPDASPLVGLVYCYWCHSPLWHNRNVSRGHEYFYYRCPNIQKHERASMIPADQAEKAVADSFLDQAGDLPVMERVWVPGDSTERELRDAVAALDELTEVLGTLGSATARQRITRQITALDERIRDLEAQPVREARWEYKQLGGTYREAWESLGEAERWQLIMKSGMTFAFGLTDRGRGPNSVWVSSVYTPEPLKQTLVSGVTQRRTLADADPHRDVNSADHTKSLPEHWATMRKHGVEGIEVHEVKSVFVSKPGERVEIPHWLHEVGISEITNDDDVRIIWTQDGRGWYQHSDGEWIEVPLGELEE

SEQ ID NO: 100 - protein, 509 aa, synthetic construct
MLRIAIYSRKSVETDTGESIQNQIKLCKEYFKRQDPNCIFEIFEDEGYSGGNINRPSFQRMMELVKIKQFDIVAVYKIDRIARNIVDFVNTYDELDNIGVKLVSITEGFDPSTPAGKMMMLLLASFAEMERMNIAQRVKDNMRELAKMGRWSGGTPPKGYTTKKVIENGKKITYLDLIDDEAYIIKDAFKLYAEGYSTYKINKHFKEKGIRLPQKTIQNMLNNPTYLISSKESVDFLKNKGYTVYGEPNGFGFLPYNRRPRTKGKKSWNDKSQFVGVSKHEGIIDLPLWIEVQNKLKERTVDPHPRESNFTFLSGGLLKCSCGSSMFVHPGHTRKDGSRLYYFRCMKNNGNCSNSKFLRVDYAESSILEFLESISSKEKLTEYQKKKKPRLDFSIEIKNLNKKIRDNSKAIDNLIDKLMILSNEAGKVVATKIEELTKQNNILKESLLEIERKKLLSGLEDNNLNILYNEIQNFIQTEDISLRRLKIKNIIKYITYNPQNDSLQVELVD

SEQ ID NO: 101 - protein, 497 aa, synthetic construct
MNAIYARQSVDRADSISIESQIEFCQYEMRGEQYKVYTDRGYSGKNTDRPAFAEMMNDIENGVIGKVVVYKLDRISRSILDFSNMMEQFGKHKVEFVSTTEKFDTSSPMGRAMLNICIVFAQLERETIQKRVADAYYSRSQKSFYMGGRVPYGFRLIPTTIEGIKTSMYEINAEEAEQVQLIYELYSKPECSYGDIIRYFQAHGILKNGKPWGRTRLADILRNPIYVRADPSVYAFYRDQGAIMANDPADFIGTNGCYYYKGQDSAGRKQMNLEGNHLVLAPHEGIISSDLWLKCRVKCLEAQQIKPYQKAKNTWLAGKIKCGACGYALVDKHYSTTRSRYLLCSNKMNAKACEGPGTIYTDEFEQIIYNEMQKKMAQFKKLRRCKGKRVNPELTALNIQLTQVETEISSLMDRLSAADDTLFRYISGRIKELDGKKQELMKRISERKLHKEADYTEINNHLTMWDELSFDDKRQTVDQLIRVIYATSDSIKIEWRI

SEQ ID NO: 102 - protein, 507 aa, synthetic construct
MGRSVITYLRYSSAIQGAEGADSTRRQNDLFKQWLKKNSDAQVVASFSDEGLSGYKGKHLTGQFGDMLARIESGEFPEGTILLVESIDRIGRLEHLETEALMNRILGNGIEIHTLQDGLIYTKDALADDLGISIIQRVKAYIAHQESKQKSFRVSQKWEQRAKLALAGEQRLTKMVPGWIDPDTFKLNEHAETVRLIFKLLLDGESLHNIARHLQSNGIKSFSRRKDANGFSVHSVRTILRSETTIGTLPASQRNDRPAIPNYYEAAIDAATFNKAQEVLDKNRKGRTPASDNPLTINIFKGLFRCQCGASVHPTGTKNKYAGVYRCNNHLDGRCDVPPLKRKPFDKWVLENFLGMIDVGNDGESERKIAALQHEVEIVTARIKKATALLLEMDDIDELKIQLKELNQKRTELQTTIDSMRRKASLTDKELPQLKDIDLTTKAGRVECQLILSKHLKGLTLGKDSVTVTLQNDTEITIPTNPLPLNDGSPIFEIADKELLDIDAYQL

SEQ ID NO: 103 - protein, 548 aa, synthetic construct
MGKLLVTYIRWSTKEQDSGDSLRRQTNLIDAFYSKHKNDYYLLPAHRYVDKGKSGFHQQHKAQGSDFRRMFENVMSGAIPEGSLIVVENFDRFSRADIDTAIDDVRQILRKGVSILTLGDGELYDKSALTDPVKLIKHIIIAERAHQESLVKQKRIAQVWNHKTQLARELKKPMGKQAPGWLELSEDGSHYIVDEDKASLVNIIYDKRLSGMSMFAICKWLNEQGYPTINQRKVRISKTKKPDGNWSALSVKHILTSRSVLGYLPAKISTEDRKTVLREEIEGFYPQIVTDSKFYAVQQLLEETGKGKTSSGEHWLYVNILKGLIRCKCGLVMTPTGIRKPVYQGTYRCNGNKESRCSYGTVSRKLLDTQLCSRLFSKLSQLHDEATDTAKLDELQRRLNTVDSELEKLTETLIQLPNITQIQEALRVKQEEKDELIVQLSREKARVKSVSSLDLSGLDMESVEGRTEAQIIIKRLVKEIVVSGNEKLVDIYLHNGNMIRGFPLDGKDDHTLTLEEATDEMQSLDDMLIFGEPVTRIYPAGDMEEVDA

SEQ ID NO: 104 - protein, 500 aa, synthetic construct
MGKQNGKAYSYIRFSSKKQEQGDSVRRQIALAEHYAHANNLILSDKNFQDLGISAFKEGNRPSLGDMLEAIEQGQIEQGSTIIIESLDRLSRRGIDVTQQIIKSILQKNVFIASLVDGLLLNRDSVNDLVSVIRIALAADLAYKESEKKSKRLRETKGQQRQRALKGEVINKILPFWLERKQNKYIFSNRLATVKRIIELRQKGLGTNKIAKILNEEGHKPLRSKGWNHTTIGKTINSVALYGAYQTSETTQDRKVILLDIVENYYPAVISKEEFMLLQSDHKQNKPGYKSEHNAFAGLLKHECGGALVRKFHVASGKTYQYHVCANARDGKCNVTKNFKNIEVALYQIMKELKLEKKTSFDKTLLEERNSVKTKIQELNNMLLELPKVPLSVLQTINNLEEKLQELEEKIKHQDNIIASEKTFNINTLRETKDPQQLNMMLKRVIENIIVFNIEKRWRIKILYRNKHSQSFIWDGSNITFVSDTKKLLELVKHTPEESK

SEQ ID NO: 105 - protein, 507 aa, synthetic construct
MGRGVITYLRYSSAIQGAEGADSTRRQNDLFKQWLKKNSDAQVVASFNDEGLSGYKGKHLSGQFGDMLSRIESGEFPEGTILLVEAIDRIGRLEHLETEALMNRIIAHGIEIHTLQDGLIYTRDALSDDLGISIIQRVKSYIAHQESKQKSFRVSQKWEQRAKLALAGEQRLTRNVPGWIDPETFKLNEHAETVRLIFKLLLDGESLHNIARHLQANNISSFSRRKDANGFSVHSVRTILRSETTIGTLPASQRNDRPAILNYYEAAIDASTFNKAQEILSKNRKGRTPASDNPLTINIFKGLFRCQCGASVHPTGTKNKYAGVYRCNNHLDGRCDVPPLKRKPFDKWMIDNFLGMIDVVSDGESERKIAALQHEVETVTARIKKATALLLEMDDITELKAQVKELNQKRTELQTTIDTLKRKTSLTDKELPQLKDIDLTTKVGRVECQLILSKHLKGLTLGKDSVTVTLQNDTEIIIPTDPLPLNDGTSILEIAEKELLGIDVYQL

SEQ ID NO: 106 - protein, 510 aa, synthetic construct
MGRRAISYIRFSSERQLKGDSVRRQSKLVTDWLDKNPEFYLDSSLSFKDLGKSAFSGKHLKGGLGDFLTAIEKGLVKAGDTLLIESLDRLSRQDIDIASELLRRILRAGVDIVTLSDGEHYTRESLKDPLSLIKSILIMQRAHEESLRKSERVQAAWNRKKELISEGIKVSRRCPAWLRLNDDRRTFTIIPDKVEVVKRAFDLRLQGLSFWAITRTLNDEGHLSLNQYTPKQKGWSDTAVKKLLRNRAVIGCFTPAGREEVQGYYPAIISESLFYRVQQLNTGQYGRASVSSNPLSVNLFRGIIKCSECGATIVLGGYALKRFGMYRCPNRSANRCSAKAISRKQTDTTILYMLALCDRFETENTDTIDSLKLQREDLQRKISKMAELAIELDDMTIITEKLRDMKNALSKLNHDIEEETKRIKAITTGSLKDIDRTTKEGMIETQLLIKGVLKEIVIDAAKRRCKVTFHNGKVIDLSITENPSEDVTEAIQSLSEVTERGLIDVDEVII

SEQ ID NO: 107 - protein, 449 aa, synthetic construct
MGRTGLYVRVSTAEQEKHGYSIKVQLEKLRAFASAKDYTVVKEYIDAAQSGAKLERPGLKQLIEDVENNALDCVLVYRLDRLSRSQKDTMYLIEDVFLKNSVAFVSLQESFDTTSSFGRAMIGMLSVFAQLERDNITERLFSGRAHRAKRGFHHGGGIIPFGYRYDVETGELKRFENESNEVKAMFEMIANGKSVSSVAKEFNTYDTTIRRRIANSVYIGKIQFDGETFDGQHEPIISKELFDKANVRMNARASNLPFKRTYLLSGLIYCGKCGERCSAYESRSKHNDKEYRRAYYRCNARTWKYKQKHGRTCEQPHIRVDELEQAVMEQVKRLPLKHKVKKRAFDFKPVENKIATIDKQKERLLDLYLNEHLDNEMFNKKSKELDKSRDKLAKQLERMRMQAADSVESYQWLDGIDWDALDKDTLREVLERIIERIVIRDKDVEIYFK

SEQ ID NO: 108 - protein, 451 aa, synthetic construct
MGKRVALYMRVSTEQQAKHGDSLREQKETLYEYIEQHKDLKVVNEYVDGGISGQKINRDEFQKLLQDVKENKIDLILFTKLDRWFRNLRHYLNTQEILEKHNVSWNAVSQQYYDTTTAYGRTFIAQVMSFAELEAQIDSERIKAVMANKIAQGEVVSGKTPLGYSIENKKLVINDDAPIVIDIFNYFLSSGSLRKTVYYLGSQYGIVRDYQSVKNMLTNKKYIGELRNNKNYCPPIIDKKLFYAVQKALPKNLKTNAKRDYIFKGLLKCSDCQGSVAGQTIKARYKKKDGTESIYERTCYRCVKRRNNKLRCTNKRAFYEKNLERYIFEATKQKFEQIQINYSKKQPKIIKKKNSKKNIENKLDRLKKAYLNEVIDLEEYKKDREALMKELNEIEVEPAKIDIKNVEFILSKEFDEIYKESSEEEKNALWRSIIDNIIVFPDGNITVNFLI

SEQ ID NO: 109 - protein, 645 aa, synthetic construct
MGLRPICYERVSSIQQIEGGGGLDDQRSALEGYLDRNSDKFSNDRIFIQDRGVSAFKNSNISSESQLGIFLQDVQNRKYGEGDALIVMSLDRISRRSSWAEDTIRFIVNSGIEVHDISASTVLRKDDPHSKLIMELIQMRSHNESLMKSVRAKAAWDRKIIEAVQNGTVISNKMPMWLKNVDNRYQVIQEKADLIIRCFEWYRDGFSTGEIVKRIADPKWQMVTVSRLVRDRRLLGEHKRYNDEIIHNVYPKVIDDDLFLTANRMMDRVMLEKNKPAEDLLLESDVVQEIFQLYESGLGSGAIVKRLPKGWSTVNVLRVLRDKNVVTQKIIDNLTFERVNQKLSMNGVANRIRKDITIAQDDYITNLFPKILKCGCCGGNIAIHYNHVRTKYVICRNREERKICDAKSIQYIRIEKNILKCVKNVDFQKLMIESTGSETSVLDGLREELSSLRREESSYNDKINERKLAGKRVGIHLNDGLTEVQDRIEEIEKEIISAQTVREIPKFDFDMDEVLDPMNIELRAKVRKQLRLVLKAVKYWMFDKRIFIQLEYFNDVLSHMLVIDNKRGGGEVMYEMSIEEKKGERIYTVHENGYAVFIASVTIGTELWSLALSRTRTIDSVGNYLSLLAREGFEIFVNEDQIDWF

SEQ ID NO: 110 - protein, 524 aa, synthetic construct
MGRQVITYLRFSSKPQERGDSIRRQKGLFERWLKDNPDAKVVDEFSDEGASAYHGHHLKGDFGRMLQNIQDGKYLSGQTVLLVESETRLNRQKARNTENLVDLITGKGVDVICLESGKIYTSTNIDDLDTSIQLKIAAHIAHQQSKEKSIKVSAAWEHRAQLALEGKQQLTKNVPGWIDPDTRKLNEHSSTVVTIFDLLLSGESLHNIARYLQANNIKSFSRREKANGFSVHSVRTILRSESTVGTLAASKRNDRPAIPNYYEPAIDVATFNKAQEILSKNRVGRAPASDNPITINLFKGIIRCQCGASVHPTGVKATYQGVYRCNNVPDGRCNVPTIKRKPFDKWMLDNIVGFLERDDGNNTDKRKAEIEYQISLVTSKLKKATTLLLELDDVTELKEQVKELNIQRSNLQSELDELNQRETLSDKPLHHLSEIDLTTKAGRVEAQLILSKFVQSIELQREMIIITLRNGTVIGKSRDLSPVLSQDLMKQVVSSPSPTDIDMFSVITSDEEFRKSGKQVTKRS

SEQ ID NO: 111 - protein, 540 aa, synthetic construct
MGPKAISYIRFSTKIQSVGDSTKRQSKYINDWLKRNPDYYLDESLRFQDLGISGFSGANAKSGAFGEFLAAVESGYIEAGSVLLVESLDRVSRQDIDTAGEQLRKILRSGVEVVTLVDNEWYTKESLKDSLSMIKAMLVMERAHEESAMKSTRLRSVWAAKRERAAKGEIMSKRCAAWLKVSEDRSRFEFIPENVKAVQRVFQLRLEGLSHVKIAKQMNDEGFSTLNQFKSVTGGWSQSSVTELLSNRSVIGFKVPSKSMAVKGVSEIPNYYPSIITDEQFYSVQQLKQGSGRKPSSDLPLLTNLFKGVLRCSECGFIVVVAGVSAKRSGIYKCSMKSEGRCNSVGFSRLQTDRALVQGLLYNTNRLSLNRDNGSAIGTLQSELEQLQKQRERLIKLAMLADDTESMAKDLKALNSQIKDAEKAVSEVHQREQSSQLETISHLDLTVKKDRIESQIIIKRIVKEIRLNTAGKKCDVFLHNGLKLYNFPLDRVVDGAQWLEILPLIDGDEFDFEGFTTKPRHIALEEAPEWVKEMEEQPKQ

SEQ ID NO: 112 - protein, 600 aa, synthetic construct
MGKKIIPYGYLRVSSLEQVRSGGGLEAQDEEVRRYITQKSDVFDIDKMVMMSDEGLSAYSGRNIKEGELGRFLADVDAGLIPAGSALVCYSVDRLSRQNPWVGTQLISTLIGAGIEIHSVAENQILRSDDPVGAIMSTIYLMRANNESVIKSERAKHGYTKRLNESIANNKVLTRQMPRWLYDNDGKYAIDPNMQKVIDFTFDSYIAGQSTGYIAKKLNDMGLKYGDTSWRGSYVAKLIRDERLIGKHIRYSKQIKGVKREIIETIPDFYPVTIDTDKFHIANNMLTSVAKNIRGRTRMTYGDISILRNLFNGVIKCGVCSGETSVVQNTRRKITNGVVTYVPYKTFLRCRNRYELKTCTQGDIRYEVIERAILNHLMGLDITTLLAAPVDNKIERYRTELELCKAEEEELQAIIKERENEGKRPRPQTLKSYEDVADRIDELTQLIESHVEDNFIPHFNVDLDSITDVSNVSERSLIKKGIATIADSICYKRISDFILVEIKYRNLNDKHVLVIDNKNSEMVVNFSIEYNEENKVYICNSFVMEYDNLSCEFTVSKTTMEDYAHMMNFVDYVSDDESYNAKEFLVKNFTHIKFIDKSNE

SEQ ID NO: 113 - protein, 526 aa, synthetic construct
MGPSAFSYVRFSSGKQAKGSSEHRQRAMLGQWLEQHPSFTLSDLRFEDLGRSGFSGEHLDHGLGQLLAAIDSGAIKSGDVILVEAVDRIGRLEPLEMLPLFSRIVKAGVSVITLEDGHVYDRSSVNETSLFLLVAKIQQAHEYSNRLSRRINASYTARREKAKAGLGIKRETPVWLTTDGQLVPHVAPHIAQAFQDYADGLGERRICRKLRESGLEEFSKTNATTVRRWLKNRTAIGYWNDIPDVYPHVVDPALFYQVQQRLDAPKVDRAKPSAHYLTGLVKCAVCGRNYNYKQRKHTDPAMLCTSRARLAGEGCSNSKTYPVIVLDQVRKLTSLPFLQHAMESASSQADPSSQRLAVIDGEIGELSRKISEATKALLVLGFTPEIQESLEQLKTAREALEEERATLLLPQAEKLTTAQLEAFSNGLLDDEPMKLNHVLQTAGYSMVVHPDGSIDVDGKRFVYEGASRKEKVYKLRLIGEDKQWSLPILTPQMATYKSLFMAAVRLPGDPSEEELRRFEEAKHSER

SEQ ID NO: 114 - protein, 552 aa, synthetic construct
MGPTAYAYIRYSSKAQGEAGRDSVDRQMASIQAITKQQGIELRTENIFSDTGISGFDGSNKNKGKLKDLIDLIISQKIQDGDFVFVESIDRLSRQKMRLSKDLVYTILDRGVTLVTTIDGQMYSRAKDGMEQDIMLSVIAQRAHEESKTKSVRRKSAWNRAKKLADEEKEIFNGHNPPYGISFNKEESRFEIVEEEAQEIRDIFESLKYVGVSLTIKKVNEYSKRKWTNRNIKHLLDTKYVLGSYMAQRRDENKKKVFERYIENYYPQIVSFELFNEAVASMKNRAHRKHYGNQTVGSLNIFRHSIKCSNCNASMLFEKQTNPKGVVYPYFQCFTRKELKNGCDQPRFRFDLAFGVFLELVKFATTSSETINPNDWNNEITSVGSFHKTLFKLLTSTEKDKELEKKISHVKNLLLEQKNYQDNINKSFEAFTDGIIPAAFIKKASETEIKIEALERELAELNIESSTRNVSLLVHSYNDIIDLYKTEAGRLKINSFFTSNNIAFSFSFDQKTRMLRCKVYYKDIHVDVINKKFPLHNPLKEFGIDNLNQYFN

SEQ ID NO: 115 - protein, 462 aa, synthetic construct
MGKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNACNIDEGEVLKQFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 116 - protein, 464 aa, synthetic construct
MGKVAIYTRVSSAEQANEGYSIHEQKRKLISFCEVNDWDRYEVFSDPGVSGGSMKRPSLQKLFDRLEEFDLVLVYKLDRLTRNVRDLLEMLEVFEKNNIAFKSATELFDTTSAIGKLFITMVGAMAEWERETIRERSLIGARAAVRSGKYIKVQPFCYDLVDQKLKPNQYAEYIRFIVDKLLSGKSANEVVRLLESKKKPPGITKWNRKTVLGWMRNPILRGHTKHGDLLIKNTHEPIISEDEHSKMLDIIDKRTHKSKTKHNSIFRGVIECPQCQNKLYLVSSIQKRANGGSYEVRRYTCATCHKNKEVKDVSFNESEIEREFINTLLKKGTDNFMVNIPKPKDYDIENNKEKILEQRANYTRAWSLGYIKDEEYFVLMDETDKLLKDIEEKESPRINIELNEQQIRSVKNLLIKGFKMATAENKEELITSTVDLIKIDFIPRRLNKEGNINTVKINEIHFKY

SEQ ID NO: 117 - protein, 562 aa, synthetic construct
MPKAISYIRFSTGRQSLGSSHERQRQAVTRWLEKHPDYTLYDKPYDDLGRSGYSGDHVDNAFGHLLAAIEDGTIPKGSTILIEQIDRVGRMEPFEMFPLLSRIVNAGVDLVTLDDGITYNRQSVNNNHLFLLVAKVQAAWGYSKTLSERTKASYAIRREKAKNGEPIKRFAVAWLTTDGKLKSHLVPYVKQVFDLYISGVGKNTIANRLRASGMPELASISSPTIDAWLQNKTAIGFWNDIPGVYKPVVTPEVFMQAQKRRQEVKTQSRSRTSKNFLVGMVKCGVCNANYIIHNKEGKPNNMRCLTHHRLKDAGCTNSETIPYQVVHFIYLSSAPSWIDKAMKVIQLTDNEKRKLYLVTEREELTASILRMAKLLARTDSPELESEFDLANERRASIDIELSVLDRKADNGVESKSTSIFVGYEATLEHDRLAFHDPIQLSALLKQAGYSITVQPGRKLYVAESNVPWVYTGVARKGNTALGYRIQDGEMEYTISNVIPEAVDVQAYKNNPDGEMQHVADRSYKHVKSPTLLNPTGLRNTNVMTIEKFESANAAMQRLTSGV

SEQ ID NO: 118 - protein, 11 aa, synthetic construct
MKAVAYIRISS

SEQ ID NO: 119 - protein, 478 aa, synthetic construct
MNKVAIYVRVSTTNQAEEGYSIEEQIDKLKAYCMIKDWSVYDIYVDAGFSGSNIKRPAIQKLIKDTKRKVFDTVLVYKLDRLSRSQKDTLYLIEDVFLENKIDFVSLLENFDTSTAFGKAMVGILSVFAQLDREQIKERMQLGKLGRAKSGKPMMWAKVAYGYTYHIGTGKMTVNQSEAIIVKEVFSSYLNGRSITKLRDDLNEKYPKTPAWSYRTIRQMLDNPVYCGYNKYKGQVYPGNHAPIISKEIYNQVQDELKIRQQKAYEHNNNYRPFQSKYMLSGIAQCGYCKAPLKITLGTIRKDGTRFKRYQCVQRTPRKTKGATVYNNNEKCNSGFYEKDDIEAYVLESISKLQTDSNCIDELFNDEPEKLDRDALNKEIETLSNKISRLNDLYINNLITLDDLKTKTDTLQSKIDILKEKLEKDPALERQKNKQKMLKKLDTKDIFKMDYEEQKMLVRALINKVQVTADSIKILWKI

SEQ ID NO: 120 - protein, 504 aa, synthetic construct
MENVCIYLRKSRADEEMEKTLGHGETLSKHRKALLKIAKEKNLHIVEIKEEIVSGESLFFRPKMLELLKEVEDKKYNGVLVMDMQRLGRGDMKEQGIILETFKNAKTKIITPNKIYDLNNDFDEEYSEFEAFMSRKELKMITRRMQGGRVRSVEDGNYIASAPPYGYDIDYILKSRTLKINEHEAEGVKIIFDSYINGNGASAISEKLNNLGFKTKLGNNFSPSSVLTIIKNPVYIGKVTWKKKEIKKSKTLGKVKDTRTRDKSEWIIANGKHKPIISEEVWNNTQEVLKNKYHIPYQLTNAPINPLAGLLVCGVCGKAMVMRPSRGILRVMCVHKCGNKSVRFDYVEKAIIDSLEQYYSNKKLEVKKQKTIQNTSNEEKELILLENELSTLNKQRLSLFDFLERGIYTEDVFLERSKNIDSRINLVESEMKKISEKIKFKKTKKDTKALLQTLNNAIENYKSSSDVITKNSYLKSILNDITYIKTPEQKRNSFSITLNPKLRF

SEQ ID NO: 121 - protein, 530 aa, synthetic construct
MIAAIYSRKSKFTGKGESIENQIQLCMDYAKNLGINEFLVYEDEGFSGKSMDRPKFKEMLKDAKDKKFDYLICYRLDRISRNISDFSTLIEDLNKLNISFVSIKEQFDTSTPMGRAMMYISSVFAQLERETIAERVRDNMYELARTGRWLGGMPPYGFISTQINYYDENMNQRKMYKLKVDEDTIEIVKLIFDKYLELRSLSKLYKYMYENGIKGTRGGNLDPSALSLILKNPAYVKADKSVVDYLRKSNIDVMGDIDNIHGILTYAKNTDSPIAAVAKHKGVIDSDKWIEAQRLLNANKAKAPRAGTGSKALLSGLLKCSKCGSNMRITYKNSKSGTIYYYICGTKKSLGVSACDCRNIRSDKAESKVIDELKNKSIKSIMSSYKDSKLENSKNIKNIKTEINSINSQIKEKETYIDNLVMQLAKVTESSASTFIINKLESLNNDLSNLKSQLESLNTISMENKQVDININMLIDNLNKFNKEIDNSDINKKRLLLSTVVDYMTWDSDTDTIKVNLIGINPSNTIASGK

SEQ ID NO: 122 - protein, 461 aa, synthetic construct
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNACNIDEGEVLKHFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 123 - protein, 593 aa, synthetic construct
MLAIYARTSTDKAENSTIEQQVKAGIEFASKNNMNPKVFQDKGVSGYKIEEDENKNPFENRPAFTQMIEDIKKGTIDAVWVWEHSRISRNQYASAYIFNIFSKYKIRLYEKDKEYDLNDPNTQLLRTMLDAVAQYERQLIVKRTTRGLHNAIDNGKRSYPSLLGYRKTIKNSKGNYIWEPVESELLQVKNWFTRYKNGESLKNIVFSQNSNENKASHILKRTTHLSRTLQHYVYTGYSLNTKGLDYLKKFDNFEIDNLQMLHNPDYWVKSIPYSLEIINREDWIEVKERLRIYKEKHKKNTNRRAEKSIGTGLITCGYCGAKFFYQVQAHKRKKGLVLYPYYFHMSCLDRTCLQSPKSVSQDKIDTIFKIFVLYSTITSDSKSKFLKERLFQEDIEVKAIKEKVKILKRDHQKTETQISKFKTALETTEDVGAITVLAKQIDNTETTLTEIKNSIISGEAELQERQEAMNKTRSQLMHYSICDLLTQFFEKWNIEEQRNHLLKIVDNAVITGTTLNIKSGEYTYIFDTNKKYEFPTVVYNEMLKEAKEDIDYSSFFRNKPDDHFERRMWSILVMSESVWHICEWRDKEKQLIF

SEQ ID NO: 124 - protein, 469 aa, synthetic construct
MMKKIAVLYMRMSTDMQEHSIESQERVLMEYAKRNGYVVIRKYIDRGISGQHASKRPDFLKMIDDSETGEFQFVLIYDSSRFARNLVESLTYKSILKENCVRLISMTEPNLEDDEMSLYIDAMQGATNEIYVRKLSKSVKRGHNDRALRGDLPGDVQFGLKRLKDGSIVLDEVKAPIMRWMYEAIYYDDSSYYSICETLAAKGIKSQRGNIIDSRQVKRMLMNIKNKAYHWAEKDGKPILKKGNYPAIVDEEIFDAVQEIIAERAKHYKKNEKPAEFRKHWLSGLLVCPHCGAGYSYNTRKPPQHDAFRCGNQTRGACKKGSSILVDVAVEMVLDKLSEVYAGPLAPYVKNITVSQPEPQIDYDKEIKLLEAQLKRAKQAYLAEIDTIDEYAQNKRRIASNIKELQEAKNQAQEGAALNEPQFKVKLLNAITLLKSDCPMSEKIPAARSIIEKILVDPRNKTMDIYFFA

SEQ ID NO: 125 - protein, 532 aa, synthetic construct
MTENNNRVCCLYRVSTDKQVDFNSNHEADLPMQRKACHKFAESKGWVIVHEEQEEGVSGHKVRAAARDKLQIIKDYARRGKFDILLVFMFDRIGRIADETPFVVEWFVRNGIRVWSTQEGEQRFDNHTDKLLNYIRFWQADGESEKTSVRTRTSLRQLVEEGHFKGGNAPYGYDLVRSGRINKRKHELYELHINEQEAAVVRIVFDKYVYEGYGPQHIATYLNNSGYRARSGKCWHPSSIRGMVQNLTYTGVLRCGDARSELMPDLQIVPQEQFENAQRIRNERSVRSTAEAENRLPLNIHGKSLLAGNAYCGHCGAKLELTSSRKWRKMADGSLDDTLRIRYTCYGKLRKQTNCTGQTGYTVHILDEIIDKAVRQIFSKMRGIPKEQIVTKRYEKENTERKNHLQDLQTQRNKAEKDLLALKTEVLACIKGESVLPRGTLAEMITEQEEKLAELENLCESATEELEKTAELMDKVSRLYDELISYADLYDSANFEAKKMIVNQLIRRVDVYRGYQINISFNFDLTPYIEGE

SEQ ID NO: 126 - protein, 486 aa, synthetic construct
MKIAYARVSSREQSENSHALEQQIARLESSQVDRVIQDVESGSKNSKSPGFRELMDLVKEGKVAEIVVTRLDRLTRSLSSLQKTMEILKAHGVALVSLDDSIDTSTAAGVFHLNMLGALAEMEVGRLSERVRHGWSHLRDRRVAMNPPFGYRKENDQHVLDTTPFLCLISDRSEWSRAKISRYYIDTFLQERSIRLTLRVVYPHFGIQVYRSRRRGPHATRLIRFSPSAFNEWLTNPVLQGHLSYRRNASGNRKREDPKTWQIIPNTHEPLITAEEAAMIKQILSRNRQAKGFGSPKRRHSVSGLVFCGECRSACYHQSGCQNYARSKRLGIPQIIRRYYQCKNWRSRACPQKAMVSLDIIEEAVIAALISRAEDLAKMADTPAPTPEPLALRELRSQLADLNRMAYNPAIENAKAQIKNQIAGMELDLTHTTQESSRLGQLISALADPDFWKEGLEPNEKSQLFQDLVSQVIIKDGAVLEVKLKV

SEQ ID NO: 127 - protein, 481 aa, synthetic construct
MITTNKVAIYVRVSTTSQAEEGYSIEEQRDKLEAYCKIKDWSVYDVYTDGGFSGSNTNRPAIERLIKDAKNKKFDTILVYKLDRLSRSQKDTLYLIEDIFIKNNIAFLSLQENFDTSTPFGKAMIGLLSVFAQLEREQIKERMQLGKLGRAKSGKSMMWAKTSYGYNYHKETGTVTINPAQALAVKFIFKSYLAGRSITKLRDDLNEKFPKEIAWNYRAVRNILDNPVYCGYNQYLGEIYKGNHESIISKEDYDKTQNELKIRQRTAAENVNPRPFQAKYILSGIGQCGYCGAPLKIMLGVKRKDGSRLKKYQCHQRHPRTLRGITTYNDNKKCDSGFYYKDDLETYVLTEISKLQNDTNYLEQIFSEDNTETIDRDSYQKQIDELSKKLGRLNDLYIDDRITLEELQTKSAEFTSMRSSLETKLGNDPALKQKDRKKGMINILNQRDILTMNYEEQKVVVRSLIDKVQVTAEDIVIKWKI

SEQ ID NO: 128 - protein, 532 aa, synthetic construct
MKQAISYVRFSSDRQRHGSSVERQEGMIADWLKRHPDYEMSDLKFSDLGKSGYHGEHIKEAGGFGKLLKALEDGFIRAGDVVLVEAIDRTGRLEPMDMLTLVINPILKAGVSIITLDDNTTYSKESVNTAQIFLLVAKIQAAHGYSAALSTRVADSYKKRRKDAAKYGIVPRRITPVWLNPDGTVREDVAPWIKTAFELYVSGVGKSTIAKRMRESGVERLAKASGPGVEGWLRNKAVIGKWETLEGTPDHQIIDDIYPAIIEPSLFYKAQVHAEKMKTQRPIKTASHFLVGLVRCGECGKNYIVQNLHGKPHSMRCRTRQSQNECTNSHCVPKPVLDAIYRYTSVTAAIEAVQQQQMGVNDKEIVTREAELLAITKRVDGLVQALTQTGPIPEVIEALKQSRIEREAAESALVILRSTVVPSAGTHWQEMGKVWKLEAADAQRLAAMLKLVDYHITVAPTGEITASHSEVLYRYIGVDRALDKYKLLANGKLMLIPKGYVDDFPYHEPFQEMQSENTWDEADYDNLRQQHQ

SEQ ID NO: 129 - protein, 472 aa, synthetic construct
MEDSSNKSVGIYVRVSTDEQAKEGFSISAQKEKLKAYCVSQGWANFKFYVDEGKSAKDTHRPSLELLLRHIEQGIIDTVLVYRLDRLTRSVRDLYTLLDYFDKYNAVFRSATEVYDTGSATGRLFITLVAAMAQWERENLGERVKMGQNEKARQGQFSAPAPFGFIKEGKSLVKNHEQGEILLEIIDKVKKGYSTRQIANYLDDSGLLPIRGYRWHPGTILTLLKNPILYGSFRWGDEIIEDTHEGYISKDEFDRIQEILKERSIVKKRDSYSVFIFQSKIVCAGCGNRLASERSKYFRKKDKQYVETNNYRCQTCAQNRKPSIMGSEKKFQKALVKYMQNVTPKLEPKIPEEKKHDYEKVHQKILNLEKQRKKYQKAWSLDLMTDEEFEQLMYETKEALKSAQNELAAAHSSDSQNSQIDIERAKEIVKMFNENWSVLTNEEKRSIVQELIKHINFTKEDGEIIITHIEFY

SEQ ID NO: 130 - protein, 524 aa, synthetic construct
MPTAFSYARFSSATQKKGSSLERQRAMVARWLVAHPDYSLSDQTFQDLGKSGWKGEHIKEGGGFAELLVAVQAGLIQKGDAVLVEAIDRTGRLPVLDMLSIIVSPILRAGVSIVTLEDNLVFTEASLNEGGHIYILVGKIQAARQYSDNLSRRLTASYDSRRRLAKEGKAPKRNTPVWLTSNGDVIGEVAEQIRLAFELYTSGLGKAVIAKRMRESGVPALAKTSGPGVEGWLRNEAAIGRWNGSEVYEPIVDLSRYQLAQIEGERRKTTPTAKTATHFYVGLVKCGSCGGNFIMRTIKGVQVSMRCRKRQELKGCDNKKVIPKVVIDAVYRHTSTPAARKLVAHERRSVNEKAIIAAEAKVLELAKEIEAMVLAFSGAMAIPEVVGRIQALHAEREAAERELALLKVTVERPPTDWRVQGRVDDLGRTDPQRLAAMLRSVGYTITIDSDGRLCTSESKTVYRYAGVNRREDMYRLAVAGGKELLISKIPVEVFDEWWEAEDGDEVVTSEWDADNPDAMRSRHG

SEQ ID NO: 131 - protein, 544 aa, synthetic construct
MQEHSPSNHSSGPVRAYSYVRMSTHKQLRGDSLRRQLERSKAFADEHSLLLDDSLRDIGISAWKGRNFKTGALGRFLSMVESGEIPKGSYLLIESLDRLSREAVPDALTLFMAIINAGITIATLGDDRQIYSRDILNGDWTKLIIGLAVMSRGHEESQTKSERVRAAIHRKRENAREGKGQITGLTPAWIDAERIGANRYTFTLNHHTETVRAIYEMAARGLGATVIARKLNAEGVPAFKSKDGWYQSIIKALLSRHDVIGTFQPHRVQDGKRVPDGDPIESYFPAAIDKDLFLRVQSMRSNPGRPGRKGDMFTNLFTGLCHCSHCGGPMTMKLSRVKGNENGRYLVCSNYVRGHRCTEGNRHFRYEPFETAILDHVRELNLAEAIATTMTNEAITGINETIAALTLQLDELRRKEQRLAMALEDDNQPIDSIIDLLRQRQQERHAIEAGLQYHQQERHRLTVRHNDPAQTCDRIGQLRTAWEQADEATRYGLRSEAHAAIRELITEISFDSGSHSAIVIVANGISAYRLQDGLINGRFHAFAA

SEQ ID NO: 132 - protein, 13 aa, synthetic construct
MKGKIALYSRVST

SEQ ID NO: 133 - protein, 465 aa, synthetic construct
MTVGIYIRVSTEEQARDGFSISAQREKLKAYCVAQDWDNFKFYVDEGVSAKDTNRPQLSILLNHIQQGLITTVLVYRLDRLTRSVMDLYKLLDTFDKYNCAFKSATEVYDTSTAMGRMFITIVAALAQWERENLGERVRMGQLEKARQGEYSAKAPFGFDKNKHNKLVINEIESKVVLDMVRKIEEGYSIRQLAIHLDSYIKPIRGYKWHIRTILDILSNNAMYGAIKWSNEIIEGAHEGILTKERFIQLQKILSSRQNIKKRQTHSIFIYQMKLICPNCGNRLSSERSRYYRKKDEQHVECNQYRCQSCALNKHTTKPFATSERKVESALMNYISNLQFEQVPKINNENNELEILKKQVKKVEKQREKYQKAWSNDLMTDDEFTERMNETKILLNSAKKKLQTLEVNNHQEIDVDVIKEKVNNIKKNWFHLSPDEKKQFMSMFIENIKIDKKDGVTEVLDIEFY

SEQ ID NO: 134 - protein, 503 aa, synthetic construct
MNNRIDAIYARQSVDKKDSISIESQIEFCKYELKGGNCKEYTDKGYSGKNTDRPKFQELVRDIKRGLIAKVIVYKLDRISRSILDFANMMELFQQYDVEFVSSTEKFDTSTPMGRAMLNICIVFAQLERETIQKRVTDAYYSRSQRGFKMGGKAPYGFHTEPIKMDGINTKKLVVNPDEAANIRLIFEMYAQPTTSYGDITRYFAEQGILFYGKELIRPTLAQMLRNPVYVQADLDVYEFFKSQGTVIVNDAADFTGTNGCYLYQGRDVKPSKKNDLKDQMLVLAPHEGIVPADIWLACRKKLMNNMKIQSARKATHTWLAGKIKCGNCGYALMSIFNPSGRQYLRCTKRLDNKSCPGCGKIITSELEAVVYQQMVKKLEKHKTLTGRKKAAKANPKIAALQVELLHVDSEIEKLVDSLTGANNVLLSYVNVKIAELDGRKQELVKQIAELTVETISPGQVNQISGYLDTWDDVSFDDKRRVVDLMITTVAATSDSLNITWKI

SEQ ID NO: 135 - protein, 478 aa, synthetic construct
MNKVAIYVRVSTTNQADEGYSIDEQIDKLKAYCEIKDWVVYKVFTDAGFTGSNIDRPAMTNLISAAKKRQFDTILVYKLDRLSRSQKDTLYLIEEIFIKNGIDFLSLSENFDTSSAFGKAMIGILSVFAQLEREQIKERMMLGKVGRAKSGKTMMWAHPAYGYTYNKETSSLDIVPAEAALIKKIYELYIKGKSISKLRDYLNDNKIFVNKSVPWSHRTISYALTNPVYCGMIRYEGKLYDGLHEPIITKELFNKTQEVLAERRMEASKKNPRPFQSKYMLSGIIRCGCCNAPMKSLLGMPRKDGTRTRRYQCINRFPRKTKPVTVYNDNKKCDSGYYYMEDVEHAVLHRISTLYSDEIEASEFFEDEITFDIQKVKDEITKIESKINKLNDLYINDFISLDSLKKQSANLINEKKIIENEIEKENSKQVNNLKEDALKILATNNIHDLDYEMQSYVVKSLIDKVFVTKEDMEILFKK

SEQ ID NO: 136 - protein, 562 aa, synthetic construct
MLHCGFPCHEERAMPSAVPYIRFSSARQTTGSSAERQRQMVTQWLTQNPDYILSELTYEDLGRSGYHGEHLNDDNGFAKLLQAVEAGSIKAGDVVLVEAIDRAGRLSPMQMLKRVIIPIIEAGVSIITLDDNVTYDESSVEGGHLFLLVAKIQAAHNYSKQLSDRTKASYAIRREQAKATGKVKRHTPIWLTSEGEVIEHVAVHVRQAFELYVSGVGKTTIANRLRASGVPELATCSGPTVEAWLRNQAAIGNWEYGKDDPDKPSEIIRGVYPAVVSDELFLQAQLRKKAAATKPRERTSKHFLVGLVKCGVCGSNYIIHNKGGKPNNMRCGTYHRLKKAGCTNDETIPYQVAVYIYSETATHWVDKALQQVQLTVNDKRKLVLTTERDALTTSITNLTEKAAALNIPELWKKLEEESNRRKVVEDELAVLERTPDAGGESGFSAALSQDQMMIHDPIQLSALLKQVEYSIVVYPNKLFTVSGEVYPWLYLGPKRKPKSNVTLGYRMLYLGDEIIISPDVPVTLDWGAPTDNPVEQMRYMLRRAYKMVSAPKPYEYNDDVAE

SEQ ID NO: 137 - protein, 387 aa, synthetic construct
MSKRTRRTFSQEFKQQIVNLYLDGKPRVEIIREYELTASAFDKWVKQSKTSGSFKEKDNLTPEQKELLELRKRNQQLEMENDILKQAALIFGPKRQVIDANKHLYPISAMCRILGLSRQSYYYQSKPKKDESELEEVVAEEFIRSRKAYGSRKIKKALSKRGIKISRRKISRIMKNRGLKSSYTVAYFKIHHSTCNEAKTTNVLNRKFLRDNPLEAIVTDLTYVRVGKKWNYVCFNLDLFNREILGYSCGEHKDAVLVKKAFSRIKQPLTEVEIFHTDRGKEFDNQTIDELLTTFDINRSLSHKGCPFDNAVAESTYKSLKVEFVYQYTFETLQQLDLELFDYVNWWNHLRLHGTLGYETPVGYRNQRLAQRILDNELGCANASEAV

SEQ ID NO: 138 - protein, 581 aa, synthetic construct
MRSAIPYIRFSSARQTTGSSAERQQQMVTQWLTEHPEYTLSDLTYKDFGKSGYHGEHVKDGGGFAKLLAAVEAGDIKAGDVVLVEAIDRTGRLHPLDMLNKVITPILAAGVSIITLDDKVTYTHESAASGHLFLLVAKIQAAYGYSKQLSERTKASYAIRLEQAKEGNKVKRNTPVWLHSDGRLNDDVAPYIKQAFELYVSGVGKTAIANRLRASGVPELVKCSGPTVEAWLRNQAAIGNWEYGKDDTDKPSQIILGVYPPVISNELFLQAQHRKSAVATKPRERTSKNFLVGIVKCGVCGANLIIHNKDGKPNNMRCLTHHRLKDAGCTNKETIPYQVVHFVYLQTAPAWIDKAMKVIQLTDNEKRKLTLTTERNEVTASIQRLAKKIAKVDSVELEAEFDLVNERRAAIDIELNILGRTDDDGAESKSKSNYVGYESNLEHDRLAFRDPIQLSALLKQAGYSIVVQPGRKLYLPNDNHPWVYAGVVRKGNMTLGYRIRNSEEEFTISQAIPEVPDVRLYGNIPNGDLVHVAERSYKYAKPPTLLNSSDKHSRKGVFVLRFESADIAMEYMKSGIETDSK

SEQ ID NO: 139 - protein, 534 aa, synthetic construct
MKQAISYIRFSSARQEGGSSVERQEGMIAKWLLDHQDYELSKLNYSDLGKSGFHGEHVKEGGNFGKLLKAVMDGYIKRDDVVLVEAIDRTGRLPALQMLSDVIAPILRAGVSIITLDDNTTYTEASVGGPHLFMLVAKIQAAHEYSRTLSRRVEDSYKKRRKDAKEKGVAPKRMTPVWLNSDGTIREDVAPWIKTAFELYVSGVGKSTIAKRLRESGVERLAKASGPGVEGWLRNKAVIGKWETLIGTPEHHVIDDVYPLIIEPSLFYKAQVHAEKMKTQRPIKTAKHFLVGLVHCGECGKNYIMQNLHGKPHSMRCRTRQSQNNCSNSYVVPKPVLDAIYRYTSVTAALEAVQLQQLGVNEKEIVTREAELLTITKRVEGFVQAVNEAGPMPELLTALKQARIERESAENALVILRATVVTPPANQWREMGKVWSLEAEDAQRLSAMLRQVGYNITVGKGAIIKSSHSDVVYQYLGVDRVKDMYRVLADGEMKLIHKSQVDDYPYHEPFHEVVGEATMDETDKENLLLQYQSS

SEQ ID NO: 140 - protein, 534 aa, synthetic construct
MSTSIPEESGPNDLELRGTPAGLPPYADLVAANPNAIFVGAYSRISDDWRKNKSKKAAASRWSAGKGVANQHRRNDMNAGRHQVIVVHRYTDNDLVASRLDVFRPDFAQMLKDLKLGRTKDGYRLDGIICVHQDRLQRTDTDWEHFVHALLAKPGRLLWTPSGSSDLTDEGEIVKTGIMAVLNKAESMKKKRRIRDWHQDLILDGLPHSGPRPFGWNEDKMTLRAEEADYLAWAIRERIKGKALSTLCAEAKKRGLKGTKGGEIAPTTLSQMMTAPRVCGYRANRGTLALDENGAPIVGKWDTICKPEEWEAVCATFSPGSTYMHRGPGAPRVTGKPKTVKYLASQLLRCMNKVERDGETRICNGTLTGSPTKSARSPYTYRCGSCNKNSIAGPMVDRQITRLLLGKLGEAQITYRRPELAWHLESTLKTLADRLAGLENRWMAGEVDDEQFYRLSPGLQAEVRKLRAERARWELENAAGSEEPADIIRKWRSGELDLAQRRRILFDVFAAIQVTPGQKGSKTPNPHRLKPIWG

SEQ ID NO: 141 - protein, 519 aa, synthetic construct
MERVLMHLRKSRADLEAEARGEGETLAKHRNILLKLAKERNLNIVKIREEVGISGESLIHRPEMLETLKEVEEGLYDAVLCMDIDRLGRGNTKEQGIILEAFKNSNTKIITPLKTYDLNNEFDEEYAEFGAFMARREFKFITRRLQRGRVATVEEGNYIAPRPPYGYIIEKNNKERYLIPHPEQAPIVKMIFDWYTHDDPNVRMGASKIANELNKFCKSPTGIAWKGSTVLSILKNAVYAGRIQWRKKEEKKSITPGKKKDVRMRPKEQWIDVEGKHEPLVSMETYLKAQEILERKYHVPYQIQNGITNPLAGLVKCGICGSSMVYRPYTHQRHPHLICYNRYCTNKSSRFEYVESKIIQGLHQLLAQYKADWFKHQRPKVNDDSVDLRQKALHRLEKELNDLYKQKDNLHNLLEKGVYSIDTFLERSNILAERIDDTKKAIHDAEKALAEEVQRNKVKKDIIPTVENVLELYYKTQDPAKKNNLLKSVLDYAVYRKEKHQRDDDFTLVLYPKLPQINS

SEQ ID NO: 142 - protein, 516 aa, synthetic construct
MQDLPKQAYSYRRFSFLTQKFGSSLKRQTKLAQDYATQHGLTLSDTSFEDLGVSAYRAANASEDAGLGQFLLALREGKITTPCTLLVENLDRLSRAKIKVAMRQLWDITDQDVHVVTLVDGRIYTKDMDFEDIMLAGLIMQRAHEESETKSKRLQEKWQERRTLGKFIHKNCPFWLTPNKDRTDYEVNKYIETVHQIYAMALGGLGSRVIAQELNSSGITAPRGGLWSTATITKVLNNRAVLGEYQPKQRVIVDGVRSEKPIGSPIQNYPVILDSDYFDQVQSALRGRHKGNNRNSTKTYRNVLKHIAHCGCCNGTIRLKQQQHLYYLQCSVECKGSRPVSIRYLHDWLNEVWITSDFASVSLSDVPEAKELATLESELEKLTEAVSGLAAAYAATLDPTINSQLLETSAKKVETQTKLDDLRSELSPYNQTKAAQFERQMLVDLAFSERNEVENFVARTKLTGLLAQLKDFIIHKGQNDIVTFEIKTAQNESKTYTAVKNPYHKTRKLTGKIWNY

SEQ ID NO: 143 - protein, 464 aa, synthetic construct
MKKRAGLYIRVSTEEQVDNYSIPEQKRRLEAYCQSHDWAIAEEYIDGGFSGAKLDRPAMQKMIADSKAGNLDVVVSIKLDRLSRSQKDTLHLIEDIFLPYHVDYVSVNESFDTGTSFGRAMVGILSVFAQLEREQILERMHSGMEARAKTGLYHGSKPPYGYALEKGVLKINPTEALAVRKVFDLWLKGYSYNKISEIMEETYHGEKAWMHPSAINQLLTNPAYTGKIRFAGEVIEGQHEPIIDEITFKKANLRLETRAANRGRQTTYLLTGVIWCGNCGSRFGINMSTCNGVRYTYYTCSPNRRKKGNEGIRCCGNKPIPTKKLDPLIIEKIKRLAISKDFFEEIQKPDASPSDAIASLESAAAEIDKRIGKLMELYSMDGIPTDTLSQQINTLYAQKKDLESQISEKQSGFKQTYEDYKEVFDKVDYAFKSGTIEEQKSIVRSLIKRIDILNQEITITWNFQ

SEQ ID NO: 144 - protein, 509 aa, synthetic construct
MRVALYYRVSTKLQEDKYSLAAQKEELTKYAKSQGWNIVGEFKDVESGGKLEKKGLTALLDLVDEGGVDVVLVVDQDRLSRLDTVAWEYLKSVLRDNNVKIAEPGNIIDLNNEDDEFISDIKNLIAKREKRAIVRRMMRGKRQRMREGKGWGLPPFEYQYDRQTGKYKAKPEWKWVIPFIDNLYLNEQLGMKAISDRLNEISKTPTGKSWNEHLVHTRLVSKAFHGVMEKTFANGETISIPGIYEPLRTEETYRKIQEEREKRGEQFSVSGRKGSEKHILKRTKITCGECGRKIQIATHGTKEKPIYYAKHGRKERVDGSVCDININTVRFDKNIMTALKEILSSEEQAKKYINLDIDQNELNALKKNIKTLNKRISKLQESLDRLLDLYLAGGLKKEKYLEKQKQLESQIEIYKKELDQNELKLKTVESNMWNYEYLYEYLEVIADLEKELTPLERAQLVGKIFPTATLYREKLILTADVKGIPVDIEIPIDPDPYPWHPKKRNTSIK

SEQ ID NO: 145 - protein, 529 aa, synthetic construct
MRKVYSYMRFSRPEQAKGTSIERQSNFAEQYALEHGLELDKSLTMMDKGLSAFHGVHKTKGAFGQFLAAVTAGKVPSGSVLVVESLDRLSREDPLIAQAALTDLILSGITVVTAADNQVYSREEIQQNPFKLIMSLVVMIRANEESETKQRRSNAFLKSALNQYQANGKIRRLGSDPSWLDFNKDNDTYSFNERVEVIRRILNLYNKGIGSLKIARQLTEESILTLSGKRSAWGQTTVANIVKSHALYGMKRINVQSVEYDLEDFYPALITKSKWLALQQHRTKRSKSMHGRGEVVHLITGHGKGFTTCGYCGGGLGAQTQKQYKRSGEYSRTVTRLHCLTHKETSSCCSSVFLEPIESAVLFAACIGVEPQSLVPTVNNVNYSALIDEVENKVKHIVEQVTAGAPWDLFKDAHDKLKLEKQKLIKERDSQKPDVNQSDVQKWVNKLVELADKARANDKEARLKCRTLINSICKSIVIKLRGHDLKSEPVVTITFVTDEQFEFKVGKNGAVQFVDNGMAVNRRLKPLLA

SEQ ID NO: 146 - protein, 498 aa, synthetic construct
MLTVAYVRVSTDDQVEHSPDAQRRRCAEYAAQRNLGPVKFLSDEGQSGKDLERPAMQELVGLIEAGQVTNLIVWRMDRLSRDSGDTSRLLRLFEQHCVNVHSLNEGDLETGSASGRFTAGLHGLLAQLEREKITENIHLGNEQAVRTGFWINRAPTGYDTVDRVLVPNDDAHLIRRAFKLRAGGQSYPQIEQGTGLKYSTVRHALENKVYLGFTRLRDERFPGKHEPLVSQAEFDAAQRAHTPGRRRGKDLLSGRVRCGECGRITGIDTNGRGKPIYRCRTRGNGCAIPGRSAAGLRRAARLSVELLRTDDQLVEAIRTHFAEKTERAGAGTAEPSRAGTLGSLRSKRQKLFELLLAGKITDDFFAEQERQLTAKIEATEAHRTEAIETHRHHNALAEAFEQAAAMLRDPAFELAEIWDNASAAERRVIVEELIESVTIYADRLEVNVTGAPPLLVTLAEVGLREPGTAPVVSEDLTDFRSSGAWGLSGRTRLIDWVA

SEQ ID NO: 147 - protein, 518 aa, synthetic construct
MEINAVNHIKDVAIYLRKSRGQEEDLDKHREELVELCEKNGWRYVEYGEIGSSQKLMDRPELSRLLKDIQEDMYDAVVVMDKDRLSREGVGQAQINKILAENDCLIVTPTRVYDLTNESDMMVSEFEDLMARFEYRAIVKRLKRGKRRGAKQGKWTNGTPPIPYIYNPKFRDKTDKSIEKGEDLDSLIVDEEKLEIYRFIVDSFLNGMSPYTIAWELNKRKIASPRGSTWNNTGIRRLLKDETHLGRIIANKTTGSKKRLSTGRLDYKVNKREDWIIVENCHKAVKTKEEHEKILIEMQKRKRDSYATTGENALSGLIVCSKCGATMAQKKSKIESERYSYVEACKNFLEDGITKCDNHGGSTMYLMKEIERQLVMYRDDIEKENERVSKGGGLTKTIESEKKKKEERIIELEDELEQVTTLALAKFFTPEEAVQQKKKILDNISKLESELYALNLQTDNLGNMTRSEKVNAINKFLEVMDMPHISNSDLNRLYKTIIHSIIWDKSNPDELKVTINFL

SEQ ID NO: 148 - protein, 563 aa, synthetic construct
MKRAALLLRCSTNIQDYNRQKLELEKVANRFKLKVAKVFGEHVTGKDDIRKGNRKSVDELINACENNEIDVVLISEVSRLTRNFLYGVTLVDKFNRDYSIPIYFRDKRKWTIDVETGEVNLEFEKELRKAFEQAEEELASIRFRMASGKRDSAGLGQWFAGFPPFGYTRQKNGYLVKNEYAPIVKEMFDKYLEEGQALITTTRYIYGKYPDIKKLKSIGNTKFILNNKAYTGRAEANIYDEIDKVTDTFYFDIPAIIDEETYNKVQKKLANNRTTTPYPKAAKYLLQKLIKCSICGAFYTRLHSNKRDTVHFKCTSDKNSLTGCKSQVYLNERIVNPIIWNFVKEQLFYVGKMNAEQRLEAIAGENDNKSKAIEEQEALKISYKEQENKLTRLEDLYLDNDIDKNRYKERKSKIEKELSSIKAQMDRLTDRIRLSDFNIKRFNSTDFTEQYFKEVEADLEKQMKVLREYVKGIYPLYIDKVYCFLKVDTIEGMFNIFYEPRKAQQKCAYFIKDTLAQYHPDIRNFYTPNNTLLTDSDEVDAYYTLEEMKEICSRNGFEVRYRE

SEQ ID NO: 149 - protein, 461 aa, synthetic construct
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNTCNIDEGEVLKQFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 150 - protein, 543 aa, synthetic construct
MPKTKTPKTAARVYRAASYARLSVDDESYGTSGSVLNQHAMIRDFADANGGLSIVAEYSDDGFSGSTFENRPGWGSLLADVETGKVDCIVVKDLSRLGRNYLDVSRYLDQVFPALGVRVVAITDGYDSAAEKTPADALMLPVKNLFNDMYCRDASAKTKASLSAKRRRGEFVGAFAPYGYAKGSGPDRGRLVIDPEPAGVVRGIFDARIGGMSAAGIAAMLNESLVPSPYEYFAAKGAARSSNFCKGERTAWDARTVLRILSNETYAGTLVQGKTCKPDFRRKAVLSVDESEWDRSPGAHEAIVDPATFGLVRKLARRDMRLSPGAKRSLPLSGFLFCADCGATMARHASRCSSGKRFYYSCETHRKNRAECGMHKVWEDELSAAVMRAVHGHALAAVDGDGVVRSAEAYRHDRRAELACRLESVEGRIEHNGDLRLRMYSDYAAGVIDKAQYAELARAVDVRLQELKGEKAGIERETAQLDDVHAETWEQTLARYRDAEGLERVMVVELIDRVLVGEGGDIEVVFRFGGPVAACATRQEGVA

SEQ ID NO: 151 - protein, 544 aa, synthetic construct
MKQLNIQSSSKITALYCRLSRDDELQGTSNSILNQKMMLEKYARDNNFTNLEFFIDDGYSGTNFNRPDWSRLQSLIDEGKIGCIIVKDMSRLGRDYLRVGYYTDIVFPEADIRFIAINNGIDSNESTENDLTPFINIINEFYAKDTSKKIRAVFKAKGESGKPLATIPPYGYLKDKEDKYKWVIDEEASKVVKKIFQLCVQGYGPSQIASELIKEGIPTPTEHFDKLGINVSSPLSEIKGNWQPKTISLILEKMEYLGHTVNFKTYKKSYKSKKKLENPKEKWQIFENTHEAIIDQETFDIVQRIRQGRRVRNNLGEMPALSGMLYCADCGAKLYQVRGKGWEHEKEYFVCASYRKHKGLCTSHQIKNVQVEELLLHELKKITEYARQYEDDFVKLVQSKTQNELNKSLKESKKDLVHVKERINKLDTIIQRLYEDMVEGKLSEDRFQKLSSNYETEQCELEKKATMLEKIIHDTEQTTLNTTAFLKQVREHTTINKLTPEIIRMFVDKIIVEKPEKIEGTRTKKQTIWIYWNYIGILDIEKTA

SEQ ID NO: 152 - protein, 510 aa, synthetic construct
MRAAIVRRVSTLKEVQENSLQNQKDFFEDYVQSKGWDIAEIYTETETGTKFSRKEMNRLIADAKAKKFDIILAKELSRLARNQRLALEIKEVIEKHGIHLVTLDGAIDTTTGNTHMFGLFAWIYEQEAQRTSERIKMALETKAKKGLYKGSNPPYGYVVREGRLYVSDDGSSEIVERIFQSYLGGKGFDAIARELFEEGVPTPSMIAGKKNQTIYWHGSSVRKILENPHYTGDMVQGRQSTISVTNKSRKEKPKTEFIVVKNTHESIISGEVFESVQQLIAFNRKKSIDNPDVCSRPHQNVHLFTGVIFCPDCGSGFHYKRNGACYICGRSDKLGDKACSKHRIREDALVAIIRWDLQRLSKLLDDQSFYNTVKDKFVKAKSKLEKELKACAGKIENIKNLKSKALSKYLEESITKSDYDDYIAAQDAEIQKLLHNKEKLDSAISASVDVDVLGKIKGIVASSLEFQEINREVINRFIEKIEVTADGNVKLYYRFAGTSKILNELMTDVN

SEQ ID NO: 153 - protein, 450 aa, synthetic construct
MTALYARLSQEDTLEGDSNSIVNQKAVLSKYAADNGFSNPVFFIDDGVSGVTFDRPNFNRMIAEIEAGNVATVIVKDMSRLGRDYLKVGYYTEIFFVERDVRYIAINDGVDSAKGDNDFTPFCNLFNDFYAKDTSKKVRAIKRAQGQAGEHLTKPPYGYMVSPTDKKQWIVDEEAAAVVKQIFDLCIGGKGPMQIAKILKEDKVLTVKAHYAEKKGKTFPDNPYNWNENSIVAILERMDYCGHTVNFKSYSKSHKLKKRIPTTKEQQAIFFNTHEAIVEDAVFERVQELRANKRRPTKADRQGMFSGLVYCADCGSKLHFATCKSFNGSQDHYRCSNYKSNTGSCTAHFIREEVLKQIIWSRIFDVTALFFDDIIAFKEMMYQQRSTETEKEMKRRKREVMQAQKRIVELDRIFKRIYEDDISGAISHDRFLKLSAEYEAEQRELEEKVK

SEQ ID NO: 154 - protein, 537 aa, synthetic construct
MKQQIYNTALYLRLSRDDELQGESSSITTQKSMLRLYAKEHHLNVIDEYIDDGWSGTNFERPSFQRMIEDIEAGKINCVVTKDLSRFGRNYIMTGQYTELYFPSHNVRYIAIDDGVDSEKGESEIAPFKNIINEWVARDTSRKVKSAFRTKFAEGAHYGAYAPLGYKKHPDIKGKLLIDDETKWIVEKIFSLAYQGYGSAKITKILREEKVPTASWLNFTRYGTFAHIFEGKPESKRYEWTIAHVKAILKSEVYIGNSVHNMQSTVSFKSKKKVRKPESEWFRVENTHEPIIDKEVFYRVQEQIKSRRRQTKEKATPIFAGLVKCADCGWSMRFGTNTANKTPYSYYACSFYGQFGKGYCSMHYIRYDVLYQAILERLQYWAKAVQQDEEKVLHKIQKAGNAERIREKKKKASALKKAENRQNEIDRLFAKMYEDRACEKITERNFVMLSGKYQKEQIELEQQITSLREELSKMEQDMIGAEKWVELIKEYSVPKELTAPLLNAMIEKILIHEATTNEDNERIQEIEIYYRFIGKVD

SEQ ID NO: 155 - protein, 459 aa, synthetic construct
VVTKDLSRLGRNYIMTGQYTELYFPSHNVRYIAIDDGVDSEKGESEIAPFKNIINEWVARDTSRKVKSAFKTKFAEGAYYGAYAPLGYKKHPDIKGKLLVDEETKWIVEKIFSLAYQGYGSAKITKILREEKVPTASWLNFTRYGTFAHIFEGKPESKRYEWTIAHVKAILKSEVYIGNSVHNRQSTVSFKSKKKVRKPESEWFRVENTHEPIIDKEVFYRVQEQIKSRRRQTKEKATPIFAGLVKCADCGWSMRFATNKANKTPYSYYSCSFYGQFGKGYCSMHYIRYDVLYQAVLERLQYWAKAVQQDEEKVLNKIQKVGNAERIREKKKKASALKKAENRQNEIDRLFAKMYEDRACEKITERNFIMLSGKYQKEQIELEQQITNLREELSKMEQDMIGAEKWIELIKEYSVPKELTAPLLNAMIEKILIHEATTNEENERIQEIEIYYRFIGKVD

SEQ ID NO: 156 - protein, 560 aa, synthetic construct
MPRIRKDKMARSYAEPFWKIGLYIRLSREDDNEDESESVINQEKILRDFVDSYFEPGTYVIVDVFADDGLTGTDTARPNFKRLEGCIVRKEVNCMIIKSLARGFRNLADQQKFLEEFIPINGARFICTGTPFIDTYANPHSASGLEVPIRGMFNEQFAATTSEEIRKTFKMKRERGEFIGAFAPYGYKKDPNDKNSLIVDEEAAEVVKSIYHWFVNEGYSKMGIAKRLNQMGEPNPEAYKKKKGFKYNNPNSDKNDGLWSASTIARILQNEVYTGVMVQGRNRVISYKVHKQINVPEEEWFVVPNTHEAIIDRETFEKAQALHKRDTRTAPGKQEVYLMSGFVRCADCKKAMRRKTARDIAYYSCRSYTDKKICSKHSIRQDKLENAVLAALQMQIALVDRLAEEIERINNAPVINRESKRLSYSLKQAEKQLKQYNDASDSLYLDWKSGEITKEEYRRLKGKIAEQIQQLEANISYLKEEMQVMADGIGTDDPYLTAFLKHKNIQSLNRGIMVELVKAVWVHENGEITVDFNFADEYQRIIDYIENNHNVIQVIENKAI

SEQ ID NO: 157 - protein, 527 aa, synthetic construct
MEKAVLYLRLSKEDIDKISEGDDSASIKNQRLLLTDYALKHDYQIVDVYSDDNESGLYDDRPDFERMIQDAKLGKFSIIIAKTQARFSRNMEHIERYLHHDFPILGIRFIGVTDGVDTADSSNKKARQINGLVNEWYCEDLSKNIRSAFKAKMKDGQYLGSSCPYGYIKDPTNHNHLIVDDYAADIVREIYKLYLQGIGKGRIGRILSDRGVLIPSLYKRNVQGINYHNANAKAETHLWSYQTIHQILNNQMYLGNMVQYRTTTLSYKDKTKKLRLPSEWIIVEGTHEPIISYAIFQRVQELQKIRTKEVNTEQKYTNIFSGLLYCADCNHTMNRNYTRKGVFCGFICSTYKRHGNKAGCRSHRVDYDALCDAVLESIKLEARKILSDKDVDELKKSRLISSREEKIENEIRILENECEKLKQYKQKTYEAYLDDLITMNEYKVYIKKYDTELSDTCKKIDKLYAEKQVTDSLDQKYKEWVNMFSDYINITELTRTVVIELIKRIDVYEDGNIKIHYRFKNPYESSK

SEQ ID NO: 158 - protein, 557 aa, synthetic construct
MLQSNKITALYCRLSQEDMQAGESGSIQHQKMILQRYADEHHFLNTKFFVDDGFSGVSFEREGLQAMLQEVEAGRVATVITKDLSRLGRNYLKTGELIEIVFPENGVRYIAINDGVDTAREDNEFTPLRNWFNEFYARDTSKKIRAVKQAQAQKGERVNGEYPYGYIPDPNNRHHLIPDPETAPIVKQVFAMFVSGVRMCEIQKWLAENKVLTIGALRYQRTGQARYQRAMIAPYTWPDKTLYDILARQEYLGHTITAKTHKVSYKSKKTRKNEEEQRYFFPNTHEPLVDEETFELAQKRIATRHRPTKAAEIDIFSGLLFCAGCGHKMYYQQGVNIEPRRFSYSCGAWRNRARTGSECTSHYIRKNVLLDLVLEDMRRVLQYVKEHEQDFICKATEYGDMEARKALAQQQKELSKAQARMTELDTLFRKLYEDNALGRLTDERFVFLTSGYEDEKKSLAARIDELQQQIATVTERKRDISRFIQIVGKYSDIQELTYENVHEFIDRILIHELDRETNTRKIEIHYSFVGQVDTEQEPTQVVNHDRRNMVDVKSIAI

SEQ ID NO: 159 - DNA, 110 bp, synthetic construct
  source 1..110: mol_type = other DNA
acttaatataagggagattacttttaaattttaatagtagtagtgttacagggtacgtagtgcttgtaacactatttttatgtataaaaaaagaccacgctcataagaac

SEQ ID NO: 160 - DNA, 112 bp, synthetic construct
  source 1..112: mol_type = other DNA
tagtgacgtctgtccgcgcagtgatcgagggagtgtgtgctttgccgactggcaaggtcaagccggtctgctaggcacagagagccggtacagtcctccccatgcaacccaa

SEQ ID NO: 161 - DNA, 118 bp, synthetic construct
  source 1..118: mol_type = other DNA
cgtaaaatcaggcgatgcgccggcacatcgcaaatgtattttgactctcgttcgggttgccgagcgtgtccgaaaatatattatcagacaactcggtcaggggagcgcgtaaacgaaa

SEQ ID NO: 162 - DNA, 109 bp, synthetic construct
  source 1..109: mol_type = other DNA
gggtgtagatgactgccttcacctgcatagttaaaacggtagcagtgaagctacgatctgtcatagcgtccgagctcactgttttaggctctaaccggtgcgggacgtg

SEQ ID NO: 163 - DNA, 114 bp, synthetic construct
  source 1..114: mol_type = other DNA
gcattgcgtggtgggctggccatatcctaattgttgcacggttgcggaatgtggtggaattccgcaccggattaacaactgccaaaaaatagaaacccgcagctcacggcatat

SEQ ID NO: 164 - DNA, 117 bp, synthetic construct
  source 1..117: mol_type = other DNA
cgaaatggttggcgttgaggtcaatgattaatgtgtatagggttaacatttaaatcagtacaatcgtagacgctctacactattttctgtgtataaaaatatcgagaataaacgctt

SEQ ID NO: 165 - DNA, 105 bp, synthetic construct
  source 1..105: mol_type = other DNA
acggcgcacgtcgttccggtctgggagcctatgcacaacgagggataacggacgttctcccatgctgcgcatagggcaggcgtgcgtgcggttgcgcgtcgtagg

SEQ ID NO: 166 - DNA, 111 bp, synthetic construct
  source 1..111: mol_type = other DNA
catatagttaatgtgtggtttgtttttttgtacggaagtgtctaaaaagcgtcgcaatttgcgggggttccgtacataataatagtcattcgggtacatccatttagtgga

SEQ ID NO: 167 - DNA, 114 bp, synthetic construct
  source 1..114: mol_type = other DNA
taatgaacatagcacaacaaaaccaaaaaacagattttacaaggttttcccgtttagtgttagcgaaatactaaaacctgataaaaaccctctccagttgttttttcttgcccc

SEQ ID NO: 168 - DNA, 112 bp, synthetic construct
  source 1..112: mol_type = other DNA
atggcgacatataagcgttcgtgctttgtcgtcaccttgttggtgtaattaggttgacgccaacagggtgataacacaagaaggactttttatttcttctattatatataga

SEQ ID NO: 169 - DNA, 115 bp, synthetic construct
  source 1..115: mol_type = other DNA
tgattacgatcagtgccctgggaggcgattccggcatggcttatatccaacaccaccgagagcgctgttgtcgagcgtgtaagccaggacgaggacgagcacgcccacgggcacg

SEQ ID NO: 170 - DNA, 111 bp, synthetic construct
  source 1..111: mol_type = other DNA
aagagtgttctaaaatagaagaaaataaaaacatacacataaagacgcacgtagatacgtgagttcctacccacttgttttttactctatcttctcttgtttccaatttct

SEQ ID NO: 171 - DNA, 117 bp, synthetic construct
  source 1..117: mol_type = other DNA
cacattccaagatgtctcaaaatcagtctcacaatccccgtataggtatagtatccctcgggtgcccaaagtatattgaaaatataccaaggttgaatacctcgtcagctaggctag

SEQ ID NO: 172 - DNA, 110 bp, synthetic construct
  source 1..110: mol_type = other DNA
cacccgtcctagtactcgcatatggcgagtctacaaccgttcccatacgacaagtcgtagtacaactattgtagatgggtgtttagggtacgaaaaaagcccccaggcca

SEQ ID NO: 173 - DNA, 116 bp, synthetic construct
  source 1..116: mol_type = other DNA
cctggagaagccgattccatgtcatgtacggtagcttgttgtgtacaggttgatgttccacgagccggacagcaacctccacaaaacctgaacagcccccggcggtggtgcgaaca

SEQ ID NO: 174 - DNA, 119 bp, synthetic construct
  source 1..119: mol_type = other DNA
attcattacaggtagaacttgttgactaataattagtgtagttttacctgtgctgcacatccagaccagttaaaactccattaaaacacgtgattattttcacacaaaaaaaacctcta

SEQ ID NO: 175 - DNA, 112 bp, synthetic construct
  source 1..112: mol_type = other DNA
tcttttatgtcagcagttcttttcatcctgtttcatcttgtacgcttcgtttcgccgaagcgtacaagatggaaatcataaaccttcaaatgcgccattcgatcttgatgga

SEQ ID NO: 176 - DNA, 113 bp, synthetic construct
  source 1..113: mol_type = other DNA
cttgaagattatgtgaggcgaacatctaaagaaaaccgaatagactttattcaagggtcattgtattgtagctattcggcattgtagcattagccataacggttataagctta

SEQ ID NO: 177 - DNA, 107 bp, synthetic construct
  source 1..107: mol_type = other DNA
ggttcattgaacaggagaacaatgatatgagtgttaaggcaagaatactagtgcttttacatagctaaacacgtacattcaactacctgttaataacaggacaatat

SEQ ID NO: 178 - DNA, 104 bp, synthetic construct
  source 1..104: mol_type = other DNA
tactatataaactaaataataaatattcttgttcactttgaaactatattgtgatattgttgcaaagaagcaaaattgatactctcttatactttactggggtg

SEQ ID NO: 179 - DNA, 117 bp, synthetic construct
  source 1..117: mol_type = other DNA
atcaattgaagaagatgtaagatgaacatctaaagaaaaccgaatagacttaaatcaagggtcattgtattgtagctattcggcattgtagcattagccataacgtttataagttca

SEQ ID NO: 180 - DNA, 104 bp, synthetic construct
  source 1..104: mol_type = other DNA
aacaggtgataggattcggatgttctcatagtgtatttagggaattaatatcagtataatgggtccctaaacacatcattttaggtattgaatatgagacgggc

SEQ ID NO: 181 - DNA, 105 bp, synthetic construct
  source 1..105: mol_type = other DNA
aaaatatacttgcgattagtcgttatttttcatagacaagtaaatcaatcatgcacatggtagcatgagtgttctatgaaaaaagagggtaggagcgatcagcta

SEQ ID NO: 182 - DNA, 107 bp, synthetic construct
  source 1..107: mol_type = other DNA
taacagtaaatttccttatataatttatgtgtactaacttatatattccggaaggataatataggttagtacacgtataaaaatatcttttatattgacacaattta

SEQ ID NO: 183 - DNA, 111 bp, synthetic construct
  source 1..111: mol_type = other DNA
ccgttgaagaactacgctaaaagtattaaatcaaaatgttcccatagtatacgaaaggacgcataccataatcaatgggaacatttgaattttcgtaaaaaaaagaggcca

SEQ ID NO: 184 - DNA, 113 bp, synthetic construct
  source 1..113: mol_type = other DNA
agaaccttgaaaaactatggcttatgctacctcgccgaatagctcaaatacaatgacccttgaatataggctattcgggttttgaaggtatctggtttttattcacatcacaa

SEQ ID NO: 185 - DNA, 105 bp, synthetic construct
  source 1..105: mol_type = other DNA
taagtgatttcagtctgagagggaaagtgtatcaatagaaaggtctcttcaggattacacctttcttatgatacacttcttcaatccttcataactcatcataaa

SEQ ID NO: 186 - DNA, 108 bp, synthetic construct
  source 1..108: mol_type = other DNA
tgaattattgatgtaagaggctttttgttctttagtgttcttaacttccttaatgtctgctattgtagtgctatctacactacggttaagcgacctctctaatgaaaa

SEQ ID NO: 187 - DNA, 104 bp, synthetic construct
  source 1..104: mol_type = other DNA
gtaacgctcttcgagaaagcagattctcatatccatcttgagtcttctttctcgcaagacaacacgaaatagacacagtctcttccctagctgtacactgagcc

SEQ ID NO: 188 - DNA, 102 bp, synthetic construct
  source 1..102: mol_type = other DNA
gttcaaccgtcctagaagaccttgatgtgtgagattcacccctaccattcgagactggcaggtggtattctcacacttcctaagatctcagcaggaagcccg

SEQ ID NO: 189 - DNA, 109 bp, synthetic construct
  source 1..109: mol_type = other DNA
caccacctattaatttaggagtgtggttgtttttgttggaagtgtgtatcaggtaacagcatagttattccgaacttccaattaataaaactctatacccgtaatcttc

SEQ ID NO: 190 - DNA, 106 bp, synthetic construct
  source 1..106: mol_type = other DNA
cctattaatttatgagtgtggttggtttttaatgaatgttttgtaactattgcgttctttctagttacataacactcattaatatttgaaatgtatttcattgatt

SEQ ID NO: 191 - DNA, 108 bp, synthetic construct
  source 1..108: mol_type = other DNA
tgacagcgccatttgctgggagagagtgatggtgtaacaagcgaattacttgggatcagatcatctgaatgttacactgccaccttttcgacgaaggtgtgtggtttc

SEQ ID NO: 192 - DNA, 108 bp, synthetic construct
  source 1..108: mol_type = other DNA
aattgccatggaaaaactaaacctgtcggcaagagcctatgatcgaattttaaaagtatcaagaactattgccgatttagcatccgaagaaaatataaaatcggaaca

SEQ ID NO: 193 - DNA, 102 bp, synthetic construct
  source 1..102: mol_type = other DNA
gtacaaataaaagcatcaagacaccgataattaacaggacaatcaacatctccacaagtgtgaaagcttcaacagattttttacgtaattttttccatagtt

SEQ ID NO: 194 - DNA, 106 bp, synthetic construct
  source 1..106: mol_type = other DNA
aaaaaggagaagtttatcttcaagatccaataggggttgataaagaaggaaatgaaatttgtttaatagatgttttaagtagtgaaaaagatttagttttagaaaa

SEQ ID NO: 195 - DNA, 104 bp, synthetic construct
  source 1..104: mol_type = other DNA
taataaagaatgaaaataaatctactattttagttacccatgatatatcagaagctatttctatgtcagataaggtcgcagttttatccaaacgtcctgcatct

SEQ ID NO: 196 - DNA, 129 bp, synthetic construct
  source 1..129: mol_type = other DNA
aatttgagttttaatttatttttttctttcgcaattctaaatttttgtaacatttgtagttcctccttcattcgaaatcatcgatagttaattctgaaactctcttttcatagatatataaataatagt

SEQ ID NO: 197 - DNA, 133 bp, synthetic construct
  source 1..133: mol_type = other DNA
cggagggtgattttaaagagtttttcataattaatactttgcactctgttattttttttataatttactatttttttcaaataataactataaatatctttgcgtagacaacctttaaatttcgctcagaacc

SEQ ID NO: 198 - DNA, 102 bp, synthetic construct
  source 1..102: mol_type = other DNA
taaagggtttcacgcataagtaccaataatgtaacaacctgtactgaatgtgcttccagtacaggttgttacaccgttaggcaaaaaaataaatatccatag

SEQ ID NO: 199 - DNA, 105 bp, synthetic construct
  source 1..105: mol_type = other DNA
tgattgtcgcaggactatccgactgggggtgtgttttgataccaaccgggctcccgaacggtatcgaaacacacccccacacgaaagtgcggggacaggcttaat

SEQ ID NO: 200 - DNA, 105 bp, synthetic construct
  source 1..105: mol_type = other DNA
agttgatccaggtggaaaaggcgatcgaagttcattgaggggttctatttttgaacgagttaactgagtttaacccataccattctcagggattaagaggatatt

SEQ ID NO: 201 - DNA, 102 bp, synthetic construct
  source 1..102: mol_type = other DNA
gaaaaaaatgatgacattattgaaaaaagcgaaggttaaagctttcacacttattgagatgttggttgtcttgctcattatcagtgtgcttctcttgctctt

SEQ ID NO: 202 - DNA, 105 bp, synthetic construct
  source 1..105: mol_type = other DNA
aaatgctggacgggaggataacagaggctcagtgttacatacgatattgttgggagcaagcttttcgtaagtaacactgagatatagacgccactggctgtggct

SEQ ID NO: 203 - DNA, 102 bp, synthetic construct
  source 1..102: mol_type = other DNA
caaaatatgtgatggaagatatgaggtttttacaacaggagaatttcgtttgtctttatctcaattcaaaaaatcaagttcttcataaacatactgtgttta

SEQ ID NO: 204 - DNA, 106 bp, synthetic construct
  source 1..106: mol_type = other DNA
tcgatcaggtcgaccagcgtgctggcttcgagtttgtgcatgaggcgtttcttgtaagtgctgaccgtcttgctgcttagaaacatgccctttgcaatctccttgt

SEQ ID NO: 205 - DNA, 148 bp, synthetic construct
  source 1..148: mol_type = other DNA
caatgagatatgcgttagttagaacaataggttgtcgggcgcttctgccaattgaaaatccgcgtgtcggtggttcaaatccgcctccgggcaccattaacttgctgaaaatgctaagtttttggaaagctctttgatcccgcttgtg

SEQ ID NO: 206 - DNA, 128 bp, synthetic construct
  source 1..128: mol_type = other DNA
gcgtcatgctcatcgaacaaatctacagagcctttaagatcatgaaaggcgaagcatatcacaaataaaactaaaaaatagattgtgtataatatattttaaatataaaaaggattgattttatgtta

SEQ ID NO: 207 - DNA, 103 bp, synthetic construct
  source 1..103: mol_type = other DNA
cgaaatacatgatggaagaaatgcgttttttacaacaagagcattttgtttgtttatatttaaatacaaaaaatcaagttatacataggcaaacgatctttat

SEQ ID NO: 208 - DNA, 105 bp, synthetic construct
  source 1..105: mol_type = other DNA
gtaatcctcccgtcagaccgttaccctgtgtagtcccttgtaaactgtactttaggtcagtttacaaggaactacacgcagaccgtgaaacgggctgctgacaac

SEQ ID NO: 209 - DNA, 102 bp, synthetic construct
  source 1..102: mol_type = other DNA
cagttccaatggttctcagaaaatttcaagttaaagcatttactgttttggagagccttattgtattatcagtagtggcatttatgacgttagtattttcaa

SEQ ID NO: 210 - DNA, 106 bp, synthetic construct
  source 1..106: mol_type = other DNA
atcgcacgctcttcgtggcagggaaagccgcagtgtaacattcagagaaactggtcacaacattttcttttgttacaccacgttgatggctagccacctatgcacc

SEQ ID NO: 211 - DNA, 110 bp, synthetic construct
  source 1..110: mol_type = other DNA
agtttaatactaaaagaggtatattaattttatttaaaaattgacattaatgactcttaaccaaatgctctgcttgtgaaaaaagctttataaaactttataaataggtt

SEQ ID NO: 212 - DNA, 102 bp, synthetic construct
  source 1..102: mol_type = other DNA
gctgctcaaaagggcagcatacatcacagtgtcacttagcacatagctgttcacaggtattttatatgttacaccaccagcccattctgctggcaatactag

SEQ ID NO: 213 - DNA, 105 bp, synthetic construct
  source 1..105: mol_type = other DNA
atttgctggctgagatggtaacaggggatcagtgttacatgtgaaaacgttgggagcaagctctttgtaagtaacactgagaagtacctaacatgagagtaaccc

SEQ ID NO: 214 - DNA, 110 bp, synthetic construct
  source 1..110: mol_type = other DNA
tactccctggtgtcctcccagcaagcgcactactgggttaggatgttcactgacaccagtacagtatcagaagcataagtggcaggacgaatacccaactgaaatcagta

SEQ ID NO: 215 - DNA, 113 bp, synthetic construct
  source 1..113: mol_type = other DNA
tgattttacgctggtgctatatcctaaactcccacagataaacagttaatggtaatgaaataacattaactgtttatctgtgtttaatgccttaacttaatctagtaggaggg

SEQ ID NO: 216 - DNA, 102 bp, synthetic construct
  source 1..102: mol_type = other DNA
ttaaaaagaaaggctttaagtttgtgggcgagacgatttgctacgccttcatgcaagcagtaggcatggtcgatgaccacattgttggctgtcctaaaaagc

SEQ ID NO: 217 - DNA, 103 bp, synthetic construct
  source 1..103: mol_type = other DNA
gacacactttcgagtgtgtcttttttattacctgaataaaaccaagaactaaagtacctagttttattcaggtaagtgtaaaaaacggctaaatctagccgtt

SEQ ID NO: 218 - DNA, 105 bp, synthetic construct
  source 1..105: mol_type = other DNA
ttggaatgacaatcaacaataagacagaaataaccattaatacgattagcatttcgattaacgtaaaccccttttcattcttcattgtccttccctcctataaat

SEQ ID NO: 219 - DNA, 103 bp, synthetic construct
  source 1..103: mol_type = other DNA
cgtacatggctcctagtgtgtacgatgggaaataaccaaaagagccgtctgtccaatggatgtcttgcatacaaccatttttgaagttgccctctgtagatag

SEQ ID NO: 220 - DNA, 147 bp, synthetic construct
  source 1..147: mol_type = other DNA
tttgcgggacgcttccgacgctgtaggaccgagtggacggcctgaagctcacttctaatccgacggttgcaggttcgagtcctgccgggggcacttctaaaacccttgcaagcaaagggttcctagcgccaacagctgagctgtgca

SEQ ID NO: 221 - DNA, 106 bp, synthetic construct
  source 1..106: mol_type = other DNA
tagatatttctctttatttaaatagttggtcgtaaattaccacatgctattggggagaagtagtgtaaaaggtacttggtctgtgttattcgccatatatcaaaaa

SEQ ID NO: 222 - DNA, 106 bp, synthetic construct
  source 1..106: mol_type = other DNA
atctgtccgcccaatcggcggcagaatgtaagctgacggaattcggcttgatcaatatggatgaatttgataaatttactcctcgcaagatggcattgttgaagaa

SEQ ID NO: 223 - DNA, 131 bp, synthetic construct
  source 1..131: mol_type = other DNA
ctaagatcaatacgatgtatcttgttattacttttgcatccatttgtttgctccttttatccaaaataaaaaacgactaaataagccgtctatttgatatttatattatggtgtgttaatttatatataga

SEQ ID NO: 224 - DNA, 103 bp, synthetic construct
  source 1..103: mol_type = other DNA
ccatggatttctcagagaactccgggcgcgtttttaatatgcgcaaagggatcccttttgtcaagacgaaaaaacaagattacctgcagaaatgctccgacta

SEQ ID NO: 225 - DNA, 104 bp, synthetic construct
  source 1..104: mol_type = other DNA
cttaaccgcttttgaatgtccgtcttagttaatccctaattgaacgctccaaaaggtgacttccaatagggatttattccttttaaaattaacggcataatcgt

SEQ ID NO: 226 - DNA, 114 bp, synthetic construct
  source 1..114: mol_type = other DNA
cgtttatcgggcggtaatattttaaagtattgcgctacactcggcacccgacacatgtggagtgctgtgtgtcgctcgtatggaagtaatgattaggagccgcatttacctttc

SEQ ID NO: 227 - DNA, 104 bp, synthetic construct
  source 1..104: mol_type = other DNA
tgtcagcgttaatgataagttggtttctatacttccttatcactgtaaacatcaaggtttgcggtgataaggaagtaatttcagattaggcggtatagccccat

SEQ ID NO: 228 - DNA, 106 bp, synthetic construct
  source 1..106: mol_type = other DNA
ttaaaaaagtctgctaataaaggcaataattctatatctgggtaagaatttccactctcccacttagatactgctggtgttgacacacctataaactttgctaatt

SEQ ID NO: 229 - DNA, 122 bp, synthetic construct
  source 1..122: mol_type = other DNA
tctttgttaaattacgcaaaatttcattcccaacttcatatccaaataaatcatttatatatttaaatttattgacatccaattgtactaagtaaaaattatcttgataataattctttaaa

SEQ ID NO: 230 - DNA, 102 bp, synthetic construct
  source 1..102: mol_type = other DNA
aagccgcatggttacggcattttccgtgttgtgcatatattgacggagcagtagagccgtgtatttatgcacaacattagattttcctttgttttgagtagg

SEQ ID NO: 231 - DNA, 102 bp, synthetic construct
  source 1..102: mol_type = other DNA
agaaactttactcaaatgtatttctgttgccagtgcaaatgatgcctgtgttgctatggctgaatatatttcaggaaatgaagaggaatttgtccgtcagat

SEQ ID NO: 232 - DNA, 112 bp, synthetic construct
  source 1..112: mol_type = other DNA
tgataataaaatataacagttattaaatcccttggaatatatgaaatctcaattccagtttgccgaaatatctggctaactttcccaattttacagacattccagcccattc

SEQ ID NO: 233-245 - skipped (13 entries)

SEQ ID NO: 246 - DNA, 75 bp, synthetic construct
  source 1..75: mol_type = other DNA
atccttggggcccagactgagcacgtgatggcaaaggaaaggaagccctgcttcctccagagggcgtcgcaggac

SEQ ID NO: 247 - DNA, 75 bp, synthetic construct
  source 1..75: mol_type = other DNA
atccttggggcccagactgagcacgattgttggcttgtcgacgacggcggtctccgtcgtcaggatcattgatgg

SEQ ID NO: 248 - DNA, 75 bp, synthetic construct
  source 1..75: mol_type = other DNA
atccttggggcccagactgagcacgnntgatggcaaaggaaaggaagccctgcttcctccagagggcgtcgcagg

SEQ ID NO: 249 - DNA, 54 bp, synthetic construct
  source 1..54: mol_type = other DNA
atccttggggcccagactgggaagccctgcttcctccagagggcgtcgcaggac

SEQ ID NO: 250 - DNA, 75 bp, synthetic construct; identical to SEQ ID NO: 246
  source 1..75: mol_type = other DNA

SEQ ID NO: 251 - DNA, 75 bp, synthetic construct
  source 1..75: mol_type = other DNA
atccttggggcccagactgagcacgattgttaccgtgacctacatgctcgaagggcgtatgcgcctgatggcaga

SEQ ID NO: 252 - DNA, 104 bp, synthetic construct
  source 1..104: mol_type = other DNA
tggaggaagcagggcttcctttcctctgccatcaatgatcctgacgacggagaccgcggtggttgaccagacaaaccacgctcccaaggtgcaatcgtgcgaag

SEQ ID NO: 253 - DNA, 118 bp, synthetic construct
  source 1..118: mol_type = other DNA
tcaatccttggggcccagactgagcacgattgttaccgtgacctacatgctcgcaagacaacacgaaatagacacagtctcttccctagctgtacactgagccgcnccgccgaatgca

SEQ ID NO: 254 - DNA, 45 bp, synthetic construct
  source 1..45: mol_type = other DNA
tcgagaccgtgacctacatgctcgaagggcgtatgcgccacgaag